SEQUENCE LISTING (91 sequences)

SEQ ID NO: 1 - DNA, 20 bp, Artificial Sequence
  Description of Artificial Sequence Synthetic oligonucleotide
tatggcccacagtctaaagt

SEQ ID NO: 2 - DNA, 20 bp, Artificial Sequence
  Description of Artificial Sequence Synthetic oligonucleotide
tagctcatgcaatgcacatg

SEQ ID NO: 3 - DNA, 20 bp, Artificial Sequence
  Description of Artificial Sequence Synthetic oligonucleotide
cttaagtagtaggtacttcc

SEQ ID NO: 4 - DNA, 20 bp, Artificial Sequence
  Description of Artificial Sequence Synthetic oligonucleotide
tagattagctggagcttggc

SEQ ID NO: 5 - DNA, 20 bp, Artificial Sequence
  Description of Artificial Sequence Synthetic oligonucleotide
tcagaagtctggcacatctg

SEQ ID NO: 6 - DNA, 20 bp, Artificial Sequence
  Description of Artificial Sequence Synthetic oligonucleotide
ggccttgtgtcacaagtctc

SEQ ID NO: 7 - DNA, 20 bp, Artificial Sequence
  Description of Artificial Sequence Synthetic oligonucleotide
tagttagcactcctgctgct

SEQ ID NO: 8 - DNA, 20 bp, Artificial Sequence
  Description of Artificial Sequence Synthetic oligonucleotide
tcagactgtatgactgacat

SEQ ID NO: 9 - DNA, 20 bp, Artificial Sequence
  Description of Artificial Sequence Synthetic oligonucleotide
tttagctcatgcaatgcaca

SEQ ID NO: 10 - DNA, 20 bp, Artificial Sequence
  Description of Artificial Sequence Synthetic oligonucleotide
aggaccttattcacatggaa

SEQ ID NO: 11 - DNA, 20 bp, Artificial Sequence
  Description of Artificial Sequence Synthetic oligonucleotide
agaagtctggcacatctgtg

SEQ ID NO: 12 - DNA, 20 bp, Artificial Sequence
  Description of Artificial Sequence Synthetic oligonucleotide
ttggccttgtgtcacaagtc

SEQ ID NO: 13 - DNA, 20 bp, Artificial Sequence
  Description of Artificial Sequence Synthetic oligonucleotide
ctttgtcccaggcacagtca

SEQ ID NO: 14 - DNA, 20 bp, Artificial Sequence
  Description of Artificial Sequence Synthetic oligonucleotide
gcctgccatattgtccctgc

SEQ ID NO: 15 - DNA, 20 bp, Artificial Sequence
  Description of Artificial Sequence Synthetic oligonucleotide
cttacaactgtgcaccttga

SEQ ID NO: 16 - DNA, 20 bp, Artificial Sequence
  Description of Artificial Sequence Synthetic oligonucleotide
caaatttcttggacctgctg

SEQ ID NO: 17 - DNA, 20 bp, Artificial Sequence
  Description of Artificial Sequence Synthetic oligonucleotide
tcaagactggcccaggcata

SEQ ID NO: 18 - DNA, 20 bp, Artificial Sequence
  Description of Artificial Sequence Synthetic oligonucleotide
tgaggcaccaatacagagga

SEQ ID NO: 19 - DNA, 20 bp, Artificial Sequence
  Description of Artificial Sequence Synthetic oligonucleotide
ctgagatcacagacaaatgg

SEQ ID NO: 20 - DNA, 20 bp, Artificial Sequence
  Description of Artificial Sequence Synthetic oligonucleotide
tttcttggacctgctgaaga

SEQ ID NO: 21 - DNA, 20 bp, Artificial Sequence
  Description of Artificial Sequence Synthetic oligonucleotide
ctgaggattgtttctgaaag

SEQ ID NO: 22 - DNA, 20 bp, Artificial Sequence
  Description of Artificial Sequence Synthetic oligonucleotide
ctgtgactacagaagcaatg

SEQ ID NO: 23 - DNA, 20 bp, Artificial Sequence
  Description of Artificial Sequence Synthetic oligonucleotide
cagaaacccaagtctaattg

SEQ ID NO: 24 - DNA, 20 bp, Artificial Sequence
  Description of Artificial Sequence Synthetic oligonucleotide
aggatgaaccatgtggtgat

SEQ ID NO: 25 - DNA, 20 bp, Artificial Sequence
  Description of Artificial Sequence Synthetic oligonucleotide
gttctgcacctatcaggcag

SEQ ID NO: 26 - DNA, 20 bp, Artificial Sequence
  Description of Artificial Sequence Synthetic oligonucleotide
agttcaggctgtccccttgg

SEQ ID NO: 27 - DNA, 20 bp, Artificial Sequence
  Description of Artificial Sequence Synthetic oligonucleotide
ccagagtggtagaagagata

SEQ ID NO: 28 - DNA, 20 bp, Artificial Sequence
  Description of Artificial Sequence Synthetic oligonucleotide
gaattcctcttctgccacct

SEQ ID NO: 29 - DNA, 20 bp, Artificial Sequence
  Description of Artificial Sequence Synthetic oligonucleotide
actcagcagcaatggcaaag

SEQ ID NO: 30 - DNA, 20 bp, Artificial Sequence
  Description of Artificial Sequence Synthetic oligonucleotide
cagataggaacaatgaagag

SEQ ID NO: 31 - DNA, 20 bp, Artificial Sequence
  Description of Artificial Sequence Synthetic oligonucleotide
cccacagaaagtaatcacca

SEQ ID NO: 32 - DNA, 20 bp, Artificial Sequence
  Description of Artificial Sequence Synthetic oligonucleotide
ccaggaaagtatcttgacag

SEQ ID NO: 33 - DNA, 20 bp, Artificial Sequence
  Description of Artificial Sequence Synthetic oligonucleotide
gttgttgcccagtggtagtg

SEQ ID NO: 34 - DNA, 20 bp, Artificial Sequence
  Description of Artificial Sequence Synthetic oligonucleotide
gttcaaatttcttggacctg

SEQ ID NO: 35 - DNA, 20 bp, Artificial Sequence
  Description of Artificial Sequence Synthetic oligonucleotide
ggttgttgcccagtggtagt

SEQ ID NO: 36 - DNA, 20 bp, Artificial Sequence
  Description of Artificial Sequence Synthetic oligonucleotide
gctagttagcactcctgctg

SEQ ID NO: 37 - DNA, 20 bp, Artificial Sequence
  Description of Artificial Sequence Synthetic oligonucleotide
tagctagttagcactcctgc

SEQ ID NO: 38 - DNA, 20 bp, Artificial Sequence
  Description of Artificial Sequence Synthetic oligonucleotide
cacttacaactgtgcacctt

SEQ ID NO: 39 - DNA, 20 bp, Artificial Sequence
  Description of Artificial Sequence Synthetic oligonucleotide
gtcacttacaactgtgcacc

SEQ ID NO: 40 - DNA, 20 bp, Artificial Sequence
  Description of Artificial Sequence Synthetic oligonucleotide
gtaggaccttattcacatgg

SEQ ID NO: 41 - DNA, 20 bp, Artificial Sequence
  Description of Artificial Sequence Synthetic oligonucleotide
gagtaggaccttattcacat

SEQ ID NO: 42 - DNA, 20 bp, Artificial Sequence
  Description of Artificial Sequence Synthetic oligonucleotide
ggccttgtgtcacaagtctc

SEQ ID NO: 43 - DNA, 20 bp, Artificial Sequence
  Description of Artificial Sequence Synthetic oligonucleotide
ccttgtgtcacaagtctcag

SEQ ID NO: 44 - DNA, 20 bp, Artificial Sequence
  Description of Artificial Sequence Synthetic oligonucleotide
aagactggcccaggcataat

SEQ ID NO: 45 - DNA, 20 bp, Artificial Sequence
  Description of Artificial Sequence Synthetic oligonucleotide
gaggattgtttctgaaagag

SEQ ID NO: 46 - DNA, 21 bp, Artificial Sequence
  Description of Artificial Sequence Synthetic primer
acggacaactgcgttgatttt

SEQ ID NO: 47 - DNA, 20 bp, Artificial Sequence
  Description of Artificial Sequence Synthetic primer
acttagctgggaagcccaac

SEQ ID NO: 48 - DNA, 23 bp, Artificial Sequence
  Description of Artificial Sequence Synthetic primer
atgaatacggctacagcaacagg

SEQ ID NO: 49 - DNA, 22 bp, Artificial Sequence
  Description of Artificial Sequence Synthetic primer
ctcttgctcagtgtccttgctg

SEQ ID NO: 50 - DNA, 23 bp, Artificial Sequence
  Description of Artificial Sequence Synthetic primer
cagagtagcgaggacttgaagag

SEQ ID NO: 51 - DNA, 22 bp, Artificial Sequence
  Description of Artificial Sequence Synthetic primer
gctggttcgtctatcttgtggg

SEQ ID NO: 52 - DNA, 20 bp, Artificial Sequence
  Description of Artificial Sequence Synthetic primer
tctaaggaagtcggggaagc

SEQ ID NO: 53 - DNA, 20 bp, Artificial Sequence
  Description of Artificial Sequence Synthetic primer
ccctcgggtgtaatcagaat

SEQ ID NO: 54 - DNA, 22 bp, Artificial Sequence
  Description of Artificial Sequence Synthetic primer
gtgccatatcagtgagctctcg

SEQ ID NO: 55 - DNA, 21 bp, Artificial Sequence
  Description of Artificial Sequence Synthetic primer
aaccaaggttgagagagcaaa

SEQ ID NO: 56 - DNA, 23 bp, Artificial Sequence
  Description of Artificial Sequence Synthetic primer
tgtcacctaccaatgagagatcc

SEQ ID NO: 57 - DNA, 23 bp, Artificial Sequence
  Description of Artificial Sequence Synthetic primer
gcggtctggcagtaaaaactatc

SEQ ID NO: 58 - DNA, 23 bp, Artificial Sequence
  Description of Artificial Sequence Synthetic primer
gtgaaacagcattgctgtcactt

SEQ ID NO: 59 - DNA, 24 bp, Artificial Sequence
  Description of Artificial Sequence Synthetic primer
ctaggccacagaattgaaagatct

SEQ ID NO: 60 - DNA, 25 bp, Artificial Sequence
  Description of Artificial Sequence Synthetic primer
gtaggtggaaattctagcatcatcc

SEQ ID NO: 61 - DNA, 20 bp, Artificial Sequence
  Description of Artificial Sequence Synthetic primer
aaattgggttacaccgctga

SEQ ID NO: 62 - DNA, 20 bp, Artificial Sequence
  Description of Artificial Sequence Synthetic primer
ccacctagcctgcctgtact

SEQ ID NO: 63 - DNA, 21 bp, Artificial Sequence
  Description of Artificial Sequence Synthetic primer
ctgtatccttgggtcaagctg

SEQ ID NO: 64 - DNA, 20 bp, Artificial Sequence
  Description of Artificial Sequence Synthetic primer
ggagaagccattttccatca

SEQ ID NO: 65 - DNA, 20 bp, Artificial Sequence
  Description of Artificial Sequence Synthetic primer
acggaacgcagtaccaaaat

SEQ ID NO: 66 - DNA, 21 bp, Artificial Sequence
  Description of Artificial Sequence Synthetic primer
caaaaatccccaagaatgtga

SEQ ID NO: 67 - DNA, 20 bp, Artificial Sequence
  Description of Artificial Sequence Synthetic primer
cgttggctacccgtgatatt

SEQ ID NO: 68 - DNA, 22 bp, Artificial Sequence
  Description of Artificial Sequence Synthetic primer
tcagaagaactcgtcaagaagg

SEQ ID NO: 69 - DNA, 23 bp, Artificial Sequence
  Description of Artificial Sequence Synthetic primer
ggtaacaattttcccgccatgtg

SEQ ID NO: 70 - DNA, 23 bp, Artificial Sequence
  Description of Artificial Sequence Synthetic primer
ggaaataaacggaacgcagtacc

SEQ ID NO: 71 - DNA, 26 bp, Artificial Sequence
  Description of Artificial Sequence Synthetic primer
gactagacatgtcttaacatctgtcc

SEQ ID NO: 72 - DNA, 25 bp, Artificial Sequence
  Description of Artificial Sequence Synthetic primer
cctattgcatggacagcagcttatg

SEQ ID NO: 73 - DNA, 11372 bp, Homo sapiens
ccttcagttcttaaagcgctgcaattcgctgctgcagccatatttcttactctctcggggctggaagcttcctgactgaagatctctctgcacttggggttctttctagaacattttctagtcccccaacaccctttatggcgtatttctttaaaaaaatcacctaaattccataaaatatttttttaaattctatactttctcctagtgtcttcttgacacgtcctccatatttttttaaagaaagtatttggaatattttgaggcaatttttaatatttaaggaatttttctttggaatcatttttggttgacatctctgttttttgtggatcagttttttactcttccactctcttttctatattttgcccatcggggctgcggatacctggttttattattttttctttgcccaacggggccgtggatacctgccttttaattcttttttattcgcccatcggggccgcggatacctgctttttatttttttttccttagcccatcggggtatcggatacctgctgattcccttcccctctgaacccccaacactctggcccatcggggtgacggatatctgctttttaaaaattttctttttttggcccatcggggcttcggatacctgctttttttttttttatttttccttgcccatcggggcctcggatacctgctttaatttttgtttttctggcccatcggggccgcggatacctgctttgatttttttttttcatcgcccatcggtgctttttatggatgaaaaaatgttggttttgtgggttgttgcactctctggaatatctacacttttttttgctgctgatcatttggtggtgtgtgagtgtacctaccgctttggcagagaatgactctgcagttaagctaagggcgtgttcagattgtggaggaaaagtggccgccattttagacttgccgcataactcggcttagggctagtcgtttgtgctaagttaaactagggaggcaagatggatgatagcaggtcaggcagaggaagtcatgtgcattgcatgagctaaacctatctgaatgaattgatttggggcttgttaggagctttgcgtgattgttgtatcgggaggcagtaagaatcatcttttatcagtacaagggactagttaaaaatggaaggttaggaaagactaaggtgcagggcttaaaatggcgattttgacattgcggcattgctcagcatggcgggctgtgctttgttaggttgtccaaaatggcggatccagttctgtcgcagtgttcaagtggcgggaaggccacatcatgatgggcgaggctttgttaagtggttagcatggtggtggacatgtgcggtcacacaggaaaagatggcggctgaaggtcttgccgcagtgtaaaacatggcgggcctctttgtctttgctgtgtgcttttcgtgttgggttttgccgcagggacaatatggcaggcgttgtcatatgtatatcatggcttttgtcacgtggacatcatggcgggcttgccgcattgttaaagatggcgggttttgccgcctagtgccacgcagagcgggagaaaaggtgggatggacagtgctggattgctgcataacccaaccaattagaaatgggggtggaattgatcacagccaattagagcagaagatggaattagactgatgacacactgtccagctactcagcgaagacctgggtgaattagcatggcacttcgcagctgtctttagccagtcaggagaaagaagtggaggggccacgtgtatgtctcccagtgggcggtacaccaggtgttttcaaggtcttttcaaggacatttagcctttccacctctgtcccctcttatttgtcccctcctgtccagtgctgcctcttgcagtgctggatatctggctgtgtggtctgaacctccctccattcctctgtattggtgcctcacctaaggctaagtatacctccccccccaccccccaacccccccaactccccacccccaccccccaccccccacctccccacccccctacccccctacccccctacccccctctggtctgccctgcactgcactgttgccatgggcagtgctccaggcctgcttggtgtggacatggtggtgagccgtggcaaggaccagaatggatcacagatgatcgttggccaacaggtggcagaagaggaattcctgccttcctcaagaggaacacctaccccttggctaatgctggggtcggattttgatttatatttatcttttggatgtcagtcatacagtctgattttgtggtttgctagtgtttgaatttaagtcttaagtgactattatagaaatgtattaagaggctttatttgtagaattcactttaattacatttaatgagtttttgttttgagttccttaaaattccttaaagtttttagcttctcattacaaattccttaacctttttttggcagtagatagtcaaagtcaaatcatttctaatgttttaaaaatgtgctggtcattttctttgaaattgacttaactattttcctttgaagagtctgtagcacagaaacagtaaaaaatttaacttcatgacctaatgtaaaaaagagtgtttgaaggtttacacaggtccaggccttgctttgttcccatccttgatgctgcactaattgactaatcacctacttatcagacaggaaacttgaattgctgtggtctggtgtcctctattcagacttattatattggagtatttcaatttttcgttgtatcctgcctgcctagcatccagttcctccccagccctgctcccagcaaacccctagtctagccccagccctactcccaccccgccccagccctgccccagccccagtcccctaaccccccagccctagccccagtcccagtcctagttcctcagtcccgcccagcttctctcgaaagtcactctaattttcattgattcagtgctcaaaataagttgtccattgcttatcctattatactgggatattccgtttacccttggcattgctgatcttcagtactgactccttgaccattttcagttaatgcatacaatcccatttgtctgtgatctcaggacaaagaatttccttactcggtacgttgaagttagggaatgtcaattgagagctttctatcagagcattattgcccacaatttgagttacttatcattttctcgatcccctgcccttaaaggagaaaccatttctctgtcattgcttctgtagtcacagtcccaattttgagtagtgatcttttcttgtgtactgtgttggccacctaaaactctttgcattgagtaaaattctaattgccaataatcctacccattggattagacagcactctgaaccccatttgcattcagcagggggtcgcagacaacccgtcttttgttggacagttaaaatgctcagtcccaattgtcatagctttgcctattaaacaaaggcaccctactgcgctttttgctgtgcttctggagaatcctgctgttcttggacaattaaagaacaaagtagtaattgctaattgtctcacccattaatcatgaagactaccagtcgcccttgcatttgccttgaggcagcgctgactacctgagatttaagagtttcttaaattattgagtaaaatcccaattatccatagttctgttagttacactatggcctttgcaaacatctttgcataacagcagtgggactgactcattcttagagccccttcccttggaatattaatggatacaatagtaattattcatggttctgcgtaacagagaagacccacttatgtgtatgcctttatcattgctcctagatagtgtgaactacctaccaccttgcattaatatgtaaaacactaattgcccatagtcccactcattagtctaggatgtcctctttgccattgctgctgagttctgactacccaagtttccttctcttaaacagttgatatgcataattgcatatattcatggttctgtgcaataaaaatggattctcaccccatcccaccttctgtgggatgttgctaacgagtgcagattattcaataacagctcttgaacagttaatttgcacagttgcaattgtccagagtcctgtccattagaaagggactctgtatcctatttgcacgctacaatgtgggctgatcacccaaggactcttcttgtgcattgatgttcataattgtatttgtccacgatcttgtgcactaacccttccactccctttgtattccagcaggggacccttactactcaagacctctgtactaggacagtttatgtgcacaatcctaattgattagaactgagtcttttatatcaaggtccctgcatcatctttgctttacatcaagagggtgctggttacctaatgcccctcctccagaaattattgatgtgcaaaatgcaatttccctatctgctgttagtctggggtctcatcccctcatattccttttgtcttacagcagggggtacttgggactgttaatgcgcataattgcaattatggtcttttccattaaattaagatcccaactgctcacaccctcttagcattacagtagagggtgctaatcacaaggacatttcttttgtactgttaatgtgctacttgcatttgtccctcttcctgtgcactaaagaccccactcacttccctagtgttcagcagtggatgacctctagtcaagacctttgcactaggatagttaatgtgaaccatggcaactgatcacaacaatgtctttcagatcagatccattttatcctccttgttttacagcaagggatattaattacctatgttacctttccctgggactatgaatgtgcaaaattccaatgttcatggtctctccctttaaacctatattctaccccttttacattatagaaagggatgctggaaacccagagtccttctcttgggactcttaatgtgtatttctaattatccatgactcttaatgtgcatattttcaattgcctaattgatttcaattgtctaagacatttcaaatgtctaattgattagaactgagtcttttatatcaagctaatatctagcttttatatcaagctaatatcttgacttctcagcatcatagaagggggtactgatttcctaaagtctttcttgaatttctattatgcaaaattgccctgaggccgggtgtggtggctcacacctgtaatcccagcactttgggaggctgaggtgggaagatcccttactgccaggagtttgagaccagcctggccaacattaaaaaaaaaaaaaagtaagacaattgccctggaatcccatccccctcacacctccttggcaaagcagcaggagtgctaactagctagtgcttcttctcttatactgcttaaatgcgcataattagcagtagttgatgtgcccctatgttagagtagaatcccgcttccttgctccatttgcattactgcaggagcttctaactagcctgaattcactctcttggactgttaatgtgcatacttatatttgctgctgtacttttttaccatgtaaggaccccacccactgtatttacatcccagctggaagtacctactacttaagacccttagactagtaaagttagcgtgcataatcttaggtgttatatacacattttcagttgcatacagttgtgccttttatcaggactcctgtacttatcaaagcagagagtgctaatcaatattaagcccttctcttcgaactgtagatggcatgtaattgcagttgtcaatggtccttcaattagacttgggtttctgacctatcacaccctctttgctttattgcatggggtactattcacttaaggcccctttctcaaactgttaatgtgcctaatgacaattacatcagtatccttccttttgaaggacagcatggttggtgacacctaaggccccatttcttggcctcccaatatgtgtgattgtatttgtcgaggttgctatgcactagagaaggaaagtgctcccctcatccccacttttcccttccagcaggaagtgcccaccccataagacccttttatttggagagtctaggtgcacaattgtaagtgaccacaagcatgcatcttggacatttatgtgcgtaatcgcacactgctcattccatgtgaataaggtcctactctccgaccccttttgcaatacagaagggttgctgataacgcagtccccttttcttggcatgttgtgtgtgattataatcgtctgggatcctatgcactagaaaaggagggtcctctccacatacctcagtctcacctttcccttccagcagggagtgcccactccataagactctcacatttggacagtcaaggtgcgtaattgttaagtgaacacaaccatgcaccttagacatggatttgcataactacacacagctcaacctatctgaataaaatcctactctcagaccccttttgcagtacagcaggggtgctgatcaccaaggccctttttcctggcctggtatgcgtgtgattatgtttgtcccggttcctgtgtattagacatggaagcctcccctgccacactccacccccaatcttcctttcccttccggcagggagtgccctctccataagacgcttacgtttggacaatcaaggtgcacagttgtaagtgaccacaggcatacaccttggacattaatgtgcataaccactttgcccattccatctgaataaggtcctactctcagaccccttttgcagtacagcaggggtgctgatcaccaaggccccttttcttggcctgttatgtgcgtgattatatttgtctgggttcctgtgtattagacaaggaagccttccccccgcccccacccccactcccagtcttcctttcccttccagcagggagtgccccctccataagatcattacatttggacaatcaaggtgcacaattataagtgaccacagccatgcaccttggacattattggacattaatgtgcgtaactgcacatggcccatcccatctgaataaggtcctactctcagatgccctttgcagtacagcaggggtactgaatcaccaaggccctttttcttggcctgttatgtgtgtgattatatttatcccagtttctgtgtaatagacatgaaagcctcccctgccacaccccacctccaatcttcctttcccttccaccagggagtgtccactccatatacccttacatttggacaatcaaggtgcacaattgtaagtgagcataggcactcaccttggacatgaatgtgcataactgcacatggcccatcccatctgaataaggtcctactctcagaccctttttgcagtacagcaggggtgctgatcaccaaggccccttttcctggcctgttatgtgtgtgattatatttgttccagttcctgtgtaatagacatggaagcctcccctgccacactccacccccaatcttcctttcccttctggcaggaagtacccgctccataagacccttacatttggacagtcaaggtgcacaattgtatgtgaccacaaccatgcaccttggacataaatgtgtgtaactgcacatggcccatcccatctgaataaggtcctactctcagaccccttttgcagtacagtaggtgtgctgataaccaaggcccctcttcctggcctgttaacgtatgtgattatatttgtctgggttccagtgtataagacatggaagcctcccctgccccaccccaccctcaatcttcctttcccttctggcagggagtgccagctccataagaaccttacatttggacagtcaaggtgcacaattctaagtgaccgcagccatgcaccttggtcaataatgtgtgtaactgcacacggcctatctcatctgaataaggccttactctcagaccccttttgcagtacagcaggggtgctgataaccaaggcccattttcctggcctgttatgtgtgtgattatatttgtccaggtttctgtgtactagacaaggaagcctcctctgccccatcccatctacgcataatctttcttttcctcccagcagggagtgctcactccataagacccttacatttggacaatcaaggtgcacaattgtaagtgaccacaaccatgcatcttggaaatttatgtgcataactgcacatggcttatcctatttgaataaagtcctactctcagaccccctttgcagtatagctggggtgctgatcactgaggcctctttgcttggcttgtctatattcttgtgtactagataagggcaccttctcatggactccctttgcttttcaacaaggagtacccactactttttaagattcttatatttgtccaaagtacatggttttaattgaccacaacaatgtcccttggacattaatgtatgtaatcaccacatggttcatcctaattaaacaaagttctaccttctcaccctccatttgcagtataccagggttgctgaccccctaagtccccttttcttggcttgttgacatgcataattgcatttatgttggttcttgtgccctagacaaggatgccccacctcttttcaatagtgggtgcccactccttatgatctttacatttgaacagttaatgtgaataattgcagttgtccacaaccctatcacttctaggaccattatacctcttttgcattactgtggggtatactgtttccctccaaggccccttctggtggactatcaacatataattgaaattttcttttgtctttgtcagtagattaaggtcataccccatcacctttcctttgtagtacaacagggtgtcctgatcaaccaaagtcctgttgttttggactgttaatatgtgcaattacatttgctcctgatctgtgcactagataaggatcctacctactttcttagtgtttttagcaggtagtgcccactactcaagactgtcacttggaatgttcatgtgcacaaactcaattctctaagcatgttcctgtaccacctttgctttagagcagggggatgatattcactaagtgccccttcttttggacttaatatgcattaatgcaattgtccacctcttcttttagactaagagttgatctccacatattccccttgcatcaggggcatgttaattatgaatgaacccttttcttttaatattaatgtcataattgtatttgtggacctgtgtaggagaaaaagaccctatgttcctcccattaccctttggattgctgctgagaagtgttaactactcataatctcagctcttggacaattaatagcattaataacaattatcaagggcactgatcattagataagactcctgcttcctcgttgcttacatcgggggtactgacccactaaggccccttgtactgttaatgtgaatatttgcaattatatatgtctccttctggtagagtgggatattatgccctagtatcccctttgcattactgcaggggctgctgactactcaaaacttctcctgggactgttaataggcacaatggcagttatcaatggttttctccctccctgaccttgttaagcaagcgccccaccccacccttagtttcccatggcataataaagtataagcattggagtattccatgcacttgtctatcaaacagtggtccatactcccaacccttttgcattgcgccagtgtgtaaaatcacaggtagccatggtgtcatgctttatatacgaagtcttccctctctctgccccttgtgtgcccttggcccctttttacagactattgctcacaatctcaggtgtccatatttgcagctattaggtaagattgtgctgtctccctcttcccttccctctgccctgccccttttgcctctttgctgggtaatgttgaccagacaaggccctttctcttggacttaaacaattctcagttgcactttccttggtcccacccattatacatgaacccctctacttcctttcgcattgcttctgagtatgctgactacccaaagccccttctgtgttattaataaacacagtactgattgtcccatttttcagcccatcagtccaagatctccctaccactttggtgtgttggtgcagtgttgactatgaaaagcaggcctgaactaggtggataagccttcactcattttctttcatttattaatgatcctagtttcaattattgtcagattctggggacaagaaccattcttgcccacctgtgttactgctttactgtgcaaaatactgaaggcaagtcagacccagggagctggattgccatcctttattttgtgtttccagtgtacactataaaattgtctccccaggaaggaaggttggcactttctctgcattcttctttccagagcagattgcctggttaagaatctcttgttgtcccctttgtatattgttattgtaaagtgccaaatgccaggatacagccagaaaaattgcttattattattaaaaaaatttttttaagaaagacatctggattgtagggtggactcgataacctggtcattatttttttgaagccaaaatatccatttatactatgtacctggtgaccagtgtctctcattttaactgagggtggtgggtctgtggatagaacactgactcttgctattttaatatcaaagatattctagagtggaactcttaagaccagtatctttgtgtgggctttaccagcattcacttttagaaaaactacctaaattttataatcctttaatttcttcatctggagcacctgcccctacttatttcaagaagattgcagtaaaacgattaaatgagggaacatatgcagaggtgcttttaaaaagcatatgccaccttttttattaattattatataaaatgaagcatttaattatagtaataatttgaagtagtttgaagtaccacactgaggtgaggacttaaaaatgataagacgagttccctattttataagaaaaataagccaaaattaaatattcttttggatataaatttcaacagtgagatagctgcctagtggaaatgaataatatcccagccactagtgtacagggtgttttgtggcacaggattatgtaatatggaactgctcaagcaaataactagtcatcacaacagcagttctttgtaataactgaaaaagaatattgtttctcggagaaggatgtcaaaagatcggcccagctcagggagcagtttgccctactagctcctcggacagctgtaaagaagagtctctggctctttagaatact

SEQ ID NO: 74 - DNA, 200 bp, Homo sapiens
cggagaaggatgtcaaaagatcggcccagctcagggagcagtttgccctactagctcctcggacagctgtaaagaagagtctctggctctttagaatactgatcccattgaagataccacgctgcatgtgtccttagtagtcatgtctccttaggctcctcttggacattctgagcatgtgagacctgaggactgcaaac

SEQ ID NO: 75 - DNA, 200 bp, Homo sapiens
agctgtaaagaagagtctctggctctttagaatactgatcccattgaagataccacgctgcatgtgtccttagtagtcatgtctccttaggctcctcttggacattctgagcatgtgagacctgaggactgcaaacagctataagaggctccaaattaatcatatctttccctttgagaatctggccaagctccagctaa

SEQ ID NO: 76 - DNA, 200 bp, Homo sapiens
gctataagaggctccaaattaatcatatctttccctttgagaatctggccaagctccagctaatctacttggatgggttgccagctatctggagaaaaagatcttcctcagaagaataggcttgttgttttacagtgttagtgatccattccctttgacgatccctaggtggagatggggcatgaggatcctccagggga

SEQ ID NO: 77 - DNA, 200 bp, Homo sapiens
taccactgggcaacaaccctaggtcaggaggttctgtcaagatactttcctggtcccagataggaagataaagtctcaaaaacaaccaccacacgtcaagctcttcattgttcctatctgccaaatcattatacttcctacaagcagtgcagagagctgagtcttcagcaggtccaagaaatttgaacacactgaaggaa

SEQ ID NO: 78 - DNA, 200 bp, Homo sapiens
tcagcaggtccaagaaatttgaacacactgaaggaagtcagccttcccacctgaagatcaacatgcctggcactctagcacttgaggatagctgaatgaatgtgtatttctttgtctctttctttcttgtctttgctctttgttctctatctaaagtgtgtcttacccatttccatgtttctcttgctaatttctttcgt

SEQ ID NO: 79 - DNA, 5830 bp, Homo sapiens
ccttttcttgttctgtctacctctcctttctctgcctacctctcttttctctttgtgaactgtgattatttgttaccccttccccttctcgttcgttttaaatttcaccttttttctgagtctggcctcctttctgctgtttctactttttatctcacatttctcatttctgcatttcctttctgcctctcttgggctattctctctctcctcccctgcgtgcctcagcatctcttgctgtttgtgattttctatttcagtattaatctctgttggcttgtatttgttctctgcttcttccctttctactcacctttgagtatttcagcctcttcatgaatctatctccctctctttgatttcatgtaatctctccttaaatatttctttgcatatgtgggcaagtgtacgtgtgtgtgtgtcatgtgtggcagaggggcttcctaacccctgcctgataggtgcagaacgtcggctatcagagcaagcattgtggagcggttccttatgccaggctgccatgtgagatgatccaagaccaaaacaaggccctagactgcagtaaaacccagaactcaagtagggcagaaggtggaaggctcatatggatagaaggcccaaagtataagacagatggtttgagacttgagacccgaggactaagatggaaagcccatgttccaagatagatagaagcctcaggcctgaaaccaacaaaagcctcaagagccaagaaaacagagggtggcctgaattggaccgaaggcctgagttggatggaagtctcaaggcttgagttagaagtcttaagacctgggacaggacacatggaaggcctaagaactgagacttgtgacacaaggccaacgacctaagattagcccagggttgtagctggaagacctacaacccaaggatggaaggcccctgtcacaaagcctacctagatggatagaggacccaagcgaaaaaggtatctcaagactaacggccggaatctggaggcccatgacccagaacccaggaaggatagaagcttgaagacctggggaaatcccaagatgagaaccctaaaccctacctcttttctattgtttacacttcttactcttagatatttccagttctcctgtttatctttaagcctgattcttttgagatgtactttttgatgttgccggttacctttagattgacagtattatgcctgggccagtcttgagccagctttaaatcacagcttttacctatttgttaggctatagtgttttgtaaacttctgtttctattcacatcttctccacttgagagagacaccaaaatccagtcagtatctaatctggcttttgttaacttccctcaggagcagacattcatataggtgatactgtatttcagtcctttcttttgaccccagaagccctagactgagaagataaaatggtcaggttgttggggaaaaaaaagtgccaggctctctagagaaaaatgtgaagagatgctccaggccaatgagaagaattagacaagaaatacacagatgtgccagacttctgagaagcacctgccagcaacagcttccttctttgagcttaggtgagcaggattctggggtttgggatttctagtgatggttatggaaagggtgactgtgcctgggacaaagcgaggtcccaaggggacagcctgaactccctgctcatagtagtggccaaataatttggtggactgtgccaacgctactcctgggtttaatacccatctctaggcttaaagatgagagaacctgggactgttgagcatgtttaatactttccttgatttttttcttcctgtttatgtgggaagttgatttaaatgactgataatgtgtatgaaagcactgtaaaacataagagaaaaaccaattagtgtattggcaatcatgcagttaacatttgaaagtgcagtgtaaattgtgaagcattatgtaaatcaggggtccacagtttttctgtaaggggtcaaatcataaatactttagactgtgggccatatggtttctgttacatatttgttttttaaacaacgtttttataaggtcaaaatcattcttagtttttgagccaattggatttggcctgctgttcatagcttaccaccccctgatgtattatttgttattcagagaaaatttctgaatactactagtttccttttctgtgcctgtccctgtgctaggcactaaaaatgcaatgattattgatatctaggtgacctgaaaaaaaatagtgaatgtgctttgtaaactgtaaagcacttgtattctactgtgataagcgttgtggatacaaagaaaggagcaagcataaaaaagtgctctttcaaaaggatatagtactatgcagacacaaggaattgtttgataaatgaataaattatatgtatatttgaggccaatttgtgtttgctgctctggtaattttgagtaaaaatgcagtattccaggtatcagaaacgaaaacacatggaaactgcttttaaactttaaaatatactgaaaacataagggactaagcttgttgtggtcacctataatgtgccagataccatgctgggtgctagagctaccaaagggggaaaagtattctcatagaacaaaaaatttcagaaaggtgcatattaaagtgctttgtaaactaaagcatgatacaaatgtcaatgggctacatatttatgaatgaatgaatggatgaatgaatattaagtgcctcttacataccagctattttgggtactgtaaaatacaagattaattctcctatgtaataagaggaaagtttatcctctatactattcagatgtaaggaatgatatattgcttaattttaaacaatcaagactttactggtgaggttaagttaaattattactgatacatttttccaggtaaccaggaaagagctagtatgaggaaatgaagtaatagatgtgagatccagaccgaaagtcacttaattcagcttgcgaatgtgctttctaaattataaagcacttgtaaatgaaaaatttgatgctttctgtatgaataaaactttctgtaagctaggtattgtctctacaaaattctcattgtatagttaaaccacagtgagaagggttctataagtagttatacaaaccaagggtttaaatacctgttaaatagatcaattttgattgcctactatgtgaactcactgttaaaggcactgaaaatttatcatatttcatttagccacagccaaaaataaggcaatacctatgttagcattttgtgaactctaaggcaccatataaatgtaactgttgattttctcacttggtgctgggtactaggtttataaaattgtatgatagttattatattgtgcaaataaagtaggaaaatttgaataacaatgattatcttttgaatacgcatacgcaagggattggttgtctgaagaatgccactatagtagttatctattgtgtgccaatctcattgctaggcattggggatgcaaagataaaccatctttattgtgtcttgggtagcagaagaaaatatgtgtaaaatcaatttataatttgtaaactgccacccatatataagctatatctgctgaatgatcattgattactcttatccttagagataacaactgggggcacaaacatttattatcattattgaacctacaacagagatctatgtgtagatttacaaagcctacagttctatacagataggaatgaactattggcttactgaatggtgattactttctgtggggctcggaactacatgccctaggatataaaaatgatgttatcattatagagtgctcacagaaggaaatgaagtaatataggtgtgagatccagaccaaaagtcatttaacaagtttattcagtgatgaaaacatgggacaaatggactaatataaggcagtgtactaagctgagtagagagataaagtcctgtccagaagatacatgcttcctggcctgattgaggagatggaaaatttttgcaaaaaacaaggtgttgtggtcttccatccagtttcttaagtgctgatgataaaagtgaattagacccaccttgacctggcctacagaagtaaaggagtaaaaataaatgcctcaggcgtgctttttgattcatttgataaacaaagcatcttttatgtggaatataccattctgggtcctgaggataagagagatgagggcattagatcactgacagctgaagatagaagaacatctttggtttgattgtttaaataatatttcaatgcctattctctgcaaggtactatgtttcgtaaattaaataggtctggcccagaagacccactcaattgcctttgagattaaaaaaaaaaaaaaaaagaaagaaaaatgcaagtttctttcaaaataaagagacatttttcctagtttcaggaatcccccaaatcacttcctcattggcttagtttaaagccaggagactgataaaagggctcagggtttgttctttaattcattaactaaacattctgcttttattacagttaaatggttcaagatgtaacaactagttttaaaggtatttgctcattggtctggcttagagacaggaagacatatgagcaataaaaaaaagattcttttgcatttaccaatttagtaaaaatttattaaaactgaataaagtgctgttcttaagtgcttgaaagacgtaaaccaaagtgcactttatctcatttatcttatggtggaaacacaggaacaaattctctaagagactgtgtttctttagttgagaagaaacttcattgagtagctgtgatatgttcgatactaaggaaaaactaaacagatcacctttgacatgcgttgtagagtgggaataagagagggctttttattttttcgttcatacgagtattgatgaagatgatactaaatgctaaatgaaatatatctgctccaaaaggcatttattctgacttggagatgcaacaaaaacacaaaaatggaatgaagtgatactcttcatcaaacagaagtgactgttatctcaaccattttgttaaatcctaaacagaaaacaaaaaaaatcatgacgaaaagacacttgcttattaattggcttggaaagtagaatataggagaaaggttactgtttattttttttcatgtattcattcattctacaaatatattcgggtgccaataggtacttggtataaggtttttggccccagagacatgggaaaaaaatgcatgccttcccagagaatgcctaatactttccttttggcttgttttcttgttaggggcatggcttagtccctaaataacattgtgtggtttaattcctactccgtatctcttctaccactctggccactacgataagcaggtagctgggttttgtagtgagcttgctccttaagttacaggaactctccttataatagacacttcattttcctagtccatccctcatgaaaaatgactgaccactgctgggcagcaggagggatgatgaccaactaattcccaaaccccagtctcattggtaccagccttggggaaccacctacacttgagccacaattggttttgaagtgcatttacaaggtttgtctattttcagttctttactttttacatgctgacacatacatacactgcctaaatagatctctttcagaaacaatcctcagataacgcatagcaaaatggagatggagacatgatttctcatgcaacagcttctctaattataccttagaaatgttctcctttttatcatcaaatctgctcaaga

SEQ ID NO: 80 - DNA, 20 bp, Artificial Sequence
  Description of Artificial Sequence Synthetic oligonucleotide
tcaagttgaggccagaagga

SEQ ID NO: 81 - DNA, 19 bp, Artificial Sequence
  Description of Artificial Sequence Synthetic primer
accctggaggagaagagga

SEQ ID NO: 82 - DNA, 20 bp, Artificial Sequence
  Description of Artificial Sequence Synthetic primer
aggcaacgcatgaggaatta

SEQ ID NO: 83 - DNA, 22 bp, Artificial Sequence
  Description of Artificial Sequence Synthetic primer
ttgccctactagctcctcggac

SEQ ID NO: 84 - DNA, 21 bp, Artificial Sequence
  Description of Artificial Sequence Synthetic primer
ttctccagatagctggcaacc

SEQ ID NO: 85 - DNA, 20 bp, Artificial Sequence
  Description of Artificial Sequence Synthetic primer
ccacaccttcttagcccaaa

SEQ ID NO: 86 - DNA, 23 bp, Artificial Sequence
  Description of Artificial Sequence Synthetic primer
ctagaggactggggaattgtatc

SEQ ID NO: 87 - DNA, 23 bp, Artificial Sequence
  Description of Artificial Sequence Synthetic primer
cctagaggactggggaattgtac

SEQ ID NO: 88 - RNA, 19 nt, Artificial Sequence
  Description of Artificial Sequence Synthetic oligonucleotide
ugguuuacaugucgacuaa

SEQ ID NO: 89 - RNA, 19 nt, Artificial Sequence
  Description of Artificial Sequence Synthetic oligonucleotide
auaaaugaaugguggauca

SEQ ID NO: 90 - RNA, 19 nt, Artificial Sequence
  Description of Artificial Sequence Synthetic oligonucleotide
ccugagcccuaccgaauug

SEQ ID NO: 91 - RNA, 19 nt, Artificial Sequence
  Description of Artificial Sequence Synthetic oligonucleotide
ggacgacccugaccucaaa

(19) United States
(12) Patent Application Publication
(10) Pub. No.: US 2023/0374505 A1
(43) Pub. Date: Nov. 23, 2023

(54) Human XIST Antisense Oligonucleotides for X Reactivation Therapy

(71) Applicant: The General Hospital Corporation, Boston, MA (US)

(72) Inventors: Jeannie T. Lee, Boston, MA (US); Yuka Takeichi, Boston, MA (US); Thomas Dial, Boston, MA (US)

(21) Appl. No.: 18/019,720

(22) PCT Filed: Aug. 5, 2021
(86) PCT No.: PCT/US21/44824

§ 371 Date: Feb. 3, 2023

Related U.S. Application Data
(60) Provisional application No. 63/062,778, filed on Aug. 7, 2020.
(60) Provisional application No. 63/091,601, filed on Oct. 14, 2020.

(51) Int. Cl. C12N 15/113; A61P 43/00

(52) U.S. Cl. CPC C12N 15/113; A61P 43/00; C12N 2310/11; C12N 2310/315; C12N 2310/3231; C12N 2320/34; C12N 2310/341

(57) ABSTRACT

Antisense Oligonucleotides targeting XIST RNA, and the use thereof for reactivating genes on the inactive X chromosome.

### CLAIM OF PRIORITY

This application claims the benefit of U.S. Provisional Application Ser. No. 63/091,601, filed on Oct. 14, 2020 and U.S. Provisional Application Ser. No. 63/062,778, filed on Aug. 7, 2020. The entire contents of the foregoing are incorporated herein by reference.

### TECHNICAL FIELD

This invention relates to compositions of one or more inhibitors of XIST RNA and inhibitors of XIST-interacting proteins. Also described are methods of using said compositions to activate expression of one or more alleles in a cell—e.g., an inactive X-linked allele, an epigenetically silenced allele, or a hypomorphic allele. For example, described herein are methods for reactivating genes on the inactive X chromosome that include administering both of an inhibitor of XIST RNA (e.g., an antisense oligonucleotide (ASO), e.g., locked nucleic acid (LNA), that targets XIST RNA), and an inhibitor of XIST-interacting protein, e.g., a chromatin-modifying protein, e.g., a small molecule.

### BACKGROUND

Diseases caused by a mutation on the mammalian X-chromosome affect males and females very differently as males have only one X chromosome and females have two. Female X-chromosomes are, however, subject to a dosage compensation mechanism in which one X-chromosomes is inactivated and is termed the inactive X (Xi), while the other X chromosome is spared inactivation and termed the active X (Xa). Because of “X-chromosome inactivation” (XCI), the female mammal is a mosaic of cells that expresses either the maternal or paternal X-chromosome (Disteche C M. Dosage compensation of the sex chromosomes. Annu Rev Genet. 2012; 46:537-560; Maduro C et al. Fitting the puzzle pieces: The bigger picture of XCI. Trends Biochem Sci. 2016; 41:138-147; Lee J T. Gracefully ageing at 50, X-chromosome inactivation becomes a paradigm for RNA and chromatin control. Nat Rev Mol Cell Biol. 2011; 12:815-826). Thus, heterozygous X-linked mutations would affect approximately half of her somatic cells. For gene products with a non-cell-autonomous function, healthy cells can usually compensate for those expressing the mutation (e.g., Factor VIII for hemophilia). With mutations in gene products that fulfill a critical role within the cells that produce them on the other hand, deficits in just half of the body's somatic cells can result in a severe disorder. One well-known example is Rett Syndrome (RTT), a human neurodevelopmental disorder caused by a mutation in the methyl-CpG-binding protein 2 (MECP2), a chromatin-associated gene product that is crucial for neuronal development (Lyst M J et al. Rett syndrome: A complex disorder with simple roots. Nat Rev Genet. 2015; 16:261-275). Whereas males do not survive, females are typically born and remain symptom free until the first or second year of life. Then, symptoms arise that include motor abnormalities, severe seizures, absent speech, and autism (Katz D M et al. Rett syndrome: Crossing the threshold to clinical translation. Trends Neurosci. 2016; 39:100-113). To date, no disease-specific therapy is available for this disorder that affects 1 in ˜10,000 girls throughout the world.

Notably, females carry a potential cure within their own cells. Every affected cell harbors a normal but dormant copy of MECP2 on the inactive X (Xi) chromosome, which may, in principle, be reactivated to alleviate disease burden. Intriguingly, in male RTT mouse models, restoring normal Mecp2 expression can reverse disease after the onset of symptoms (Giacometti E et al. Partial rescue of MeCP2 deficiency by postnatal activation of MeCP2. Proc Natl Acad Sci USA. 2007; 104:1931-1936; Guy J et al. Reversal of neurological defects in a mouse model of Rett syndrome. Science. 2007; 315:1143-1147). There are, however, two obstacles to an Xi-reactivation strategy. First, sex chromosomal dosage compensation is known to be important throughout development and life: perturbing XCI by a germline deletion of the master regulator XIST resulted in inviable female embryos (Marahrens Y et al. XIST-deficient mice are defective in dosage compensation but not spermatogenesis. Genes Dev. 1997; 11:156-166); an epiblast-specific deletion of XIST caused severely reduced female fitness (Yang L et al. Female mice lacking XIST RNA show partial dosage compensation and survive to term. Genes Dev. 2016; 30:1747-1760); and a conditional deletion of XIST in blood caused fully penetrant hematologic cancers (Yildirim E et al. XIST RNA is a potent suppressor of hematologic cancer in mice. Cell. 2013; 152:727-742). Perturbing dosage balance via Xi-reactivation could therefore have untoward physiological consequences. On the other hand, loss of XIST and partial reactivation occurs naturally in lymphocytes (Wang J et al. Unusual maintenance of X chromosome inactivation predisposes female lymphocytes for increased expression from the inactive X. Proc Natl Acad Sci USA. 2016; 113:E2029-E2038) and Xi-reactivation may therefore be tolerated in vivo under controlled circumstances. A second challenge is that the Xi has been difficult to reactivate via pharmacological means due to multiple parallel mechanisms of epigenetic silencing (Disteche C M. Dosage compensation of the sex chromosomes. Annu Rev Genet. 2012; 46:537-560; Csankovszki G et al. Synergism of XIST RNA, DNA methylation, and histone hypoacetylation in maintaining X chromosome inactivation. J Cell Biol. 2001; 153:773-784). Progress has been made in recent years, however. Several siRNA screens identified several factors regulating Xi stability, but no overlap of candidates was observed between them (Bhatnagar S et al. Genetic and pharmacological reactivation of the mammalian inactive X chromosome. Proc Natl Acad Sci USA. 2014; 111:12591-12598; Chan K M et al. Diverse factors are involved in maintaining X chromosome inactivation. Proc Natl Acad Sci USA. 2011; 108:16699-16704), perhaps because the screens were not saturating. Others have identified the TGF-β pathway (Sripathy S et al. Screen for reactivation of MeCP2 on the inactive X chromosome identifies the BMP/TGF-β superfamily as a regulator of XIST expression. Proc Natl Acad Sci USA. 2017; 114:1619-1624), a synergism between Aurora kinase and DNA methylation in a primed small molecule screen (Lessing D et al. A high-throughput small molecule screen identifies synergism between DNA methylation and Aurora kinase pathways for X reactivation. Proc Natl Acad Sci USA. 2016; 113:14366-14371), as well as a synergism between a ribonucleotide reductase subunit (RM2) and 5-aza-2′-deoxycytidine (Minkovsky A et al. A high-throughput screen of inactive X chromosome reactivation identifies the enhancement of DNA demethylation by 5-aza-2′-dC upon inhibition of ribonucleotide reductase. Epigenetics Chromatin. 2015; 8:42). In a more direct approach, an XIST RNA proteomic screen identified more than a hundred interacting proteins and demonstrated that de-repression of the Xi could be achieved robustly only when 2-3 interactors were targeted simultaneously (Minajigi A et al. A comprehensive XIST interactome reveals cohesin repulsion and an RNA-directed chromosome conformation. Science. 2015; 349:aab2276-12).

### SUMMARY

This disclosure is based in part on the surprising discovery that expression of an inactive X-linked allele is increased in a human cell by providing an isolated antisense oligonucleotide (ASO) targeting XIST RNA and an inhibitor of an XIST-interacting protein.

In some embodiments, described herein is an isolated antisense oligonucleotide (ASO) comprising 12-50 consecutive nucleotides of SEQ ID NOs:1-45, or 12-50 consecutive nucleotides of a sequence at or within 100, 75, 50, 25, 10, or 5 nts of the binding sites for ASOs comprising SEQ ID NOs:1-45 in XIST, e.g. in SEQ ID NO:73-79, as shown in FIG. 7. In some embodiments, described herein is an isolated antisense oligonucleotide (ASO) comprising SEQ ID NOs:1-45. In some embodiments, an isolated antisense oligonucleotide (ASO) comprises at least one modification. In some embodiments, the at least one modification comprises one or more modified bonds or bases. In some embodiments, the modified bases comprise at least one ribonucleotide, at least one deoxyribonucleotide, or at least one bridged nucleotide, wherein the bridged nucleotide is a locked nucleic acid (LNA) nucleotide, a 2′-O-Ethyl (cEt) modified nucleotide, 2′-O-methoxy ethyl (MOE) nucleotide, or a 2′-0,4′-C-ethylene (ENA) modified nucleotide. In some embodiments, the modified bonds comprise phosphorothioate internucleotide linkages between at least two nucleotides, or between all nucleotides. In some embodiments, the ASO is a gapmer or mixmer. In some embodiments, the ASO comprises unmodified deoxyribonucleosides in the center flanked by 5′ and 3′ terminal modified (e.g., bridged, locked) nucleosides. In some embodiments, comprises unmodified deoxyribonucleosides in the center flanked by 5′ and 3′ terminal modified (e.g., bridged, locked) nucleosides directs RNAse-H-mediated cleavage of a target XIST transcript. In some embodiments, the locked nucleosides comprise a methylene bridge between the 2′-oxygen and the 4′-carbon. In some embodiments, there are 1, 2, 3, 4, 5, 6, 7, 8, 9, or 10 modified (e.g., bridged, locked) nucleosides at the 3′ end. In some embodiments, there are 1, 2, 3, 4, 5, 6, 7, 8, 9, or 10 modified (e.g., bridged, locked) nucleosides at the 5′ end. In some embodiments, the modified nucleosides at the 3′ end and/or the 5′ end are 2′-O-methoxy ethyl (MOE) nucleotides. In some embodiments, the ASO comprises 1, 2, 3, 4, 5, 6, 7, 8, 9, or 10 2′-MOE nucleosides at the 3′ end and/or 1, 2, 3, 4, 5, 6, 7, 8, 9, or 10 2′-MOE nucleosides at the 5′ end.

In some embodiments, described herein is an isolated antisense oligonucleotide (ASO) comprising 12-50 nucleotides that binds to 12-50 consecutive nucleotides of SEQ ID NO:73-79. In some embodiments, described herein is an isolated ASO comprising 12-50 nucleotides that binds to 12-50 consecutive nucleotides of human XIST RNA Exon 1, 4, 5, or 6; preferably Exons 4, 5, or 6; preferably Exon 6; preferably the first 1-2500 nucleotides of Exon 6; preferably nucleotides 600-1750 of Exon 6. In some embodiments, described herein is an isolated ASO comprising 12-50 nucleotides that binds to 12-50 consecutive nucleotides of human XIST RNA repeat A, repeat B, repeat C, repeat D, or repeat E. In some embodiments, an isolated antisense oligonucleotide (ASO) comprises at least one modification. In some embodiments, the at least one modification comprises one or more modified bonds or bases. In some embodiments, the modified bases comprise at least one ribonucleotide, at least one deoxyribonucleotide, or at least one bridged nucleotide, wherein the bridged nucleotide is a locked nucleic acid (LNA) nucleotide, a 2′-O-Ethyl (cEt) modified nucleotide, 2′-O-methoxy ethyl (MOE) nucleotide, or a 2′-O,4′-C-ethylene (ENA) modified nucleotide. In some embodiments, the modified bonds comprise phosphorothioate internucleotide linkages between at least two nucleotides, or between all nucleotides. In some embodiments, the ASO is a gapmer or mixmer. In some embodiments, the ASO comprises unmodified deoxyribonucleosides in the center flanked by 5′ and 3′ terminal modified (e.g., bridged, locked) nucleosides. In some embodiments, comprises unmodified deoxyribonucleosides in the center flanked by 5′ and 3′ terminal modified (e.g., bridged, locked) nucleosides directs RNAse-H-mediated cleavage of a target XIST transcript. In some embodiments, the locked nucleosides comprise a methylene bridge between the 2′-oxygen and the 4′-carbon. In some embodiments, there are 1, 2, 3, 4, 5, 6, 7, 8, 9, or 10 modified (e.g., bridged, locked) nucleosides at the 3′ end. In some embodiments, there are 1, 2, 3, 4, 5, 6, 7, 8, 9, or 10 modified (e.g., bridged, locked) nucleosides at the 5′ end. In some embodiments, the modified nucleosides at the 3′ end and/or the 5′ end are 2′-O-methoxy ethyl (MOE) nucleotides. In some embodiments, the ASO comprises 1, 2, 3, 4, 5, 6, 7, 8, 9, or 10 2′-MOE nucleosides at the 3′ end and/or 1, 2, 3, 4, 5, 6, 7, 8, 9, or 10 2′-MOE nucleosides at the 5′ end.

In some embodiments, described herein is a composition comprising any described isolated ASO and a pharmaceutically acceptable carrier.

In some embodiments, described herein is a composition comprising any described isolated ASO and an inhibitor of an XIST-interacting protein. In some embodiments, the inhibitor of an XIST-interacting protein inhibits a protein shown in Table 2, e.g., SMC1a; SMC3; WAPL; RAD21; KIF4; PDS5a/b; CTCF; TOP1; TOP2a; TOP2b; SMARCA4 (BRG1); SMARCA5; SMARCC1; SMARCC2; SMARCB1; RING1a/b (PRC1); PRC2 (EZH2, SUZ12, RBBP7, RBBP4, EED); AURKB; SPEN/MINT/SHARP; DNMT1; DNMT3a/3b; SmcHD1; CTCF; MYEF2; ELAV1; SUN2; Lamin-B Receptor (LBR); LAP; hnRPU/SAF-A; hnRPK; hnRPC; PTBP2; RALY; MATRIN3; MacroH2A; and ATRX. In some embodiments, the inhibitor of an XIST-interacting protein is a small molecule inhibitor or an inhibitory nucleic acid that targets a gene encoding the XIST-interacting protein. In some embodiments, the inhibitor of an XIST-interacting protein is an inhibitor of DNA methyltransferase (DNMT). In some embodiments, the inhibitor of DNMT is RG 108, 5-azacytidine (also called “azacytidine” throughout the application), decitabine (also called “5-Aza-2′-deoxycytidine” throughout the application), Zebularine, procainamide, procaine, psammaplin A, sinefungin, temozolomide, OM173-alphaA, DNMT3A-binding protein, theaflavin 3,3′-digallate, 1-Hydrazinophthalazine, SGI-1027, hydralazine, NSC14778, Olsalazine, Nanaomycin, SID 49645275, Δ²-isoxazoline, epigallocatechin-3-gallate (EGCG), MG98, SGI-110, SGI-1027, SW155246, SW15524601, SW155246-2, or DZNep, an ASO targeting an DNMT, e.g. SEQ ID NO: 80, TCAAGTTGAGGCCAGAAGGA, or an siRNA targeting DNMT, optionally comprising SEQ ID NOs: 88-91.

In some embodiments, described herein is a composition comprising any described isolated ASO and more than one inhibitor of an XIST-interacting protein. In some embodiments, the more than one inhibitor of an XIST-interacting protein are inhibitors of DNA methyltransferase (DNMT), wherein the inhibitors of DNMT are selected from RG108, 5-azacytidine, decitabine, Zebularine, procainamide, procaine, hydralazine, NSC14778, Olsalazine, Nanaomycin, SID 49645275, Δ²-isoxazoline, epigallocatechin-3-gallate (EGCG), MG98, SGI-110, SGI-1027, SW155246, SW15524601, SW155246-2, or DZNep, an ASO targeting DNMT, e.g. SEQ ID NO: 80, TCAAGTTGAGGCCAGAAGGA, or an siRNA targeting DNMT, optionally comprising SEQ ID NOs: 88-91. In some embodiments, the more than one inhibitor of an XIST-interacting protein are at least two of RG108, 5-azacytidine, decitabine, Zebularine, procainamide, procaine, psammaplin A, sinefungin, temozolomide, OM173-alphaA, DNMT3A-binding protein, theaflavin 3,3′-digallate, 1-Hydrazinophthalazine, SGI-1027, hydralazine, NSC14778, Olsalazine, Nanaomycin, SID 49645275, Δ²-isoxazoline, epigallocatechin-3-gallate (EGCG), MG98, SGI-110, SGI-1027, SW155246, SW15524601, SW155246-2, or DZNep, an ASO targeting an DNMT, e.g. SEQ ID NO: 80, TCAAGTTGAGGCCAGAAGGA, and/or an siRNA targeting an DNMT, e.g. any one of SEQ ID NOs: 88-91.

In some embodiments, described herein is a method of increasing expression of an inactive X-linked allele in a cell, preferably a cell of a female heterozygous subject or male hemizygous subject, the method comprising administering to the cell an isolated ASO of the disclosure and an inhibitor of an XIST-interacting protein. In some embodiments, the inhibitor of an XIST-interacting protein inhibits a protein shown in Table 2, e.g., SMC1a; SMC3; WAPL; RAD21; KIF4; PD5Sa/b; CTCF; TOP1; TOP2a; TOP2b; SMARCA4 (BRG1); SMARCA5; SMARCC1; SMARCC2; SMARCB1; RING1a/b (PRC1); PRC2 (EZH2, SUZ12, RBBP7, RBBP4, EED); AURKB; SPEN/MINT/SHARP; DNMT1; DNMT3a/3b; SmcHD1; CTCF; MYEF2; ELAV1; SUN2; Lamin-B Receptor (LBR); LAP; hnRPU/SAF-A; hnRPK; hnRPC; PTBP2; RALY; MATRIN3; MacroH2A; and ATRX. In some embodiments, the inhibitor of an XIST-interacting protein is a small molecule inhibitor of the XIST-interacting protein, or an inhibitory nucleic acid that targets a gene encoding the XIST-interacting protein. In some embodiments, the inhibitor of an XIST-interacting protein is an inhibitor of DNA methyltransferase (DNMT). In some embodiments, the inhibitor of DNMT is RG108, 5-azacytidine, decitabine, Zebularine, procainamide, procaine, psammaplin A, sinefungin, temozolomide, OM173-alphaA, DNMT3A-binding protein, theaflavin 3,3′-digallate, 1-Hydrazinophthalazine, SGI-1027, hydralazine, NSC14778, Olsalazine, Nanaomycin, SID 49645275, Δ²-isoxazoline, epigallocatechin-3-gallate (EGCG), MG98, SGI-110, SGI-1027, SW155246, SW15524601, SW155246-2, DZNep, an ASO targeting DNMT, e.g. SEQ ID NO: 80, TCAAGTTGAGGCCAGAAGGA and/or an siRNA targeting an DNMT, e.g. any one of SEQ ID NOs: 88-91. In some embodiments, the cell is in a living subject. In some embodiments, the cell is in or from a subject who has an X-linked disorder. In some embodiments, the X-linked disorder is Rett syndrome or CDKL5 deficiency disorder. In some embodiments, the X-linked disorder is any one of the disorders listing in Table 4.

In some embodiments, described herein is an inhibitory ASO of the disclosure, or a composition of the disclosure, for use in increasing expression of an inactive X-linked allele in a cell, preferably a cell of a female heterozygous subject, and further preferably wherein the inactive X-linked allele is associated with an X-linked disorder.

In some embodiments, described herein is an inhibitor of XIST RNA and an inhibitor of an XIST-interacting protein, for use in increasing expression of an inactive X-linked allele in a cell, preferably a cell in a female heterozygous subject, and further preferably wherein the active X-linked allele is associated with an X-linked disorder.

In some embodiments, described herein is an inhibitor of XIST RNA and an inhibitor of an XIST-interacting protein, for use in treating an X-linked disorder in a female heterozygous or male hemizygous subject.

In some embodiments, the X-linked disorder is any one of the disorders listing in Table 4. In some embodiments, the X-linked disorder is Rett syndrome or CDKL5 deficiency disorder.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1C. Identifying human XIST ASOs. (A) Schematic representation of the locations of different tested ASOs on the human XIST. Conserved XIST repeat elements A-E are indicated. (B) Schematic representation of XIST ASO treatment of a human CDKL5 patient fibroblast line carrying mutation on the Xa. After 24 hours or 48 hours of treatment with 100 nM XIST ASOs (lipofectamine transfection), harvested cells underwent qPCR analysis. Negative control=HPRT MOE-based ASO. (C) qPCR results showing the fold change in XIST RNA expression in human fibroblast treated with negative control ASO (HPRT) and XIST ASO compared with negative control treated cells for 24 hours or 48 hours, normalized to RPL13a, relative to HPRT. XIST MOE-based ASO efficiently depletes XIST RNA.

FIGS. 2A-2B. A mixed modality drug that synergistically restores MECP2 protein (A) XIST LNA-based ASO efficiently depletes XIST RNA. Negative control=scrambled ASO-LNA. (B) We treated a reporter fibroblast line carrying a knocked-in Mecp2:Luciferase fusion on the Xi. After 5 days of treatment with an XIST ASO and Aza, we achieved a significant reactivation to 3-5% of what is normally expressed on the Xa.

FIG. 3. Phenotypic improvement with 5-10% MECP2 protein expression. Direct correlation between MECP2 protein levels in the brain and lifespan. X's plot females with various levels of MECP2 protein. Purple, skewed-RTT females. Red, RTT males. Green, wildtype female. Blue, wildtype male.

FIGS. 4A-4B. MECP2-GFP Upregulation in the Brain. (A) Xi-Mecp2-GFP/+ mice were injected with dyes by ICV and the brain sectioned to determine extent of dye penetration through the ventricles. (B) Mecp2-GFP/+ mice were injected by ICV at 5 weeks with 200 ug XIST ASO+15 ug 5-Aza-dC combination and sacrificed after 4 weeks. Whole brain lysates were immunoblotted for GFP (MECP2 surrogate). Lanes 2-4 show 1/10 and 1/125 dilutions of lysate of untreated Xa-Mecp2-GFP/+ control mice, suggesting that reactivation in lane 9 was ˜1%. M, markers.

FIG. 5. MECP2 Upregulation in the Brain in the disease model, TsixACpG-RTT females. Mecp2-GFP/+ mice in lanes 7-9 were injected by ICV at 5 weeks with 200 ug XIST ASO+15 ug 5-Aza-dC combination and sacrificed after 4 weeks. Whole brain lysates were immunoblotted for MECP2. The mice showed good reactivation, with mouse 4 showing strongest reactivation.

FIGS. 6A-6B. XIST ASO (ASO-6B) and DNMTi co-treatment in CDKL5 patient fibroblast. (A) A schematic of treatment of a human CDKL5 patient fibroblast line carrying mutation on the Xa. After 3 days of treatment with 100 nM XIST ASOs (lipofectamine transfection) and 0.5 uM Aza (added every 2 days), harvested cells underwent qPCR analysis. Negative control=HPRT MOE-based ASO. qPCR results show percentage of CDKL5 wt allele reactivation after 3 days treatment with or without 100 nM ASO-6B and 0.5 uM Aza. CDKL5 wt reactivation was normalized to CDKL5mut expression from the Xa chromosome, normalized to RPL13a (B) qPCR results show percentage of CDKL5 wt allele reactivation after 3 days treatment with or without 100 nM ASO-6C and 0.5 uM Aza. CDKL5 wt reactivation was normalized to CDKL5mut expression from the Xa chromosome, normalized to RPL13a.

FIG. 7. The sequences of exons 1-6 are shown in SEQ ID NOs:73-79 (see to FIG. 7); XIST exons correspond to 601-11972 (exon 1); 15851-15914 (exon 2); 19593-20116 (exon 3); 21957-21984 (exon 4); 22080-22288 (exon 5); and 23887-33304 (exon 6) of the full length XIST sequence (GENBANK ID number: NR 001564). ASO binding sites are highlighted.

FIGS. 8A-8C. Xi reactivation in human CDKL5 patient's fibroblast with XIST ASO+/−DNMTi (Decitabine, Azacitidine, RG-108). (A) A human CDKL5 patient fibroblast line carrying mutation on the Xa was treated with ASOs to XIST and three DNMTi. On day 0, cells were treated with 20 nM of XIST ASO-6B, and 1 uM of each DNMTi every 2 days. On day 7, cells were harvested for qPCR analysis. (B) qPCR results show percentage of XIST expression after 7 days treatment with or without 20 nM ASO-6B and 1 uM of each of DNMTi (Decitabine, Azacitidine, RG-108). XIST expression was relative to untreated levels, normalized to RPL13a. (C) qPCR results show percentage of CDKL5 wt allele reactivation after 7 days treatment with or without 20 nM ASO-6B and 1 uM of each of DNMTi (Decitabine, Azacitidine, RG-108). CDKL5 wt reactivation was normalized to CDKL5mut expression from the Xa chromosome.

FIGS. 9A-9C. Xi reactivation in human CDKL5 patient's fibroblast with XIST ASO-6B and DNMT inhibitor (SGI-1027) co-treatment. (A) A human CDKL5 patient fibroblast line carrying mutation on the Xa was treated with an ASO to XIST and an DNMTi. After 5 days of treatment with 20 nM XIST ASO-6B (added once at day 0; lipofectamine transfection) and 100 nM-1.0 uM SGI-1027 (added every 2 days; days 0, 2, and 4), cells were harvested for qPCR analysis. (B) qPCR results show percentage of XIST expression after 5 days treatment with or without 20 nM ASO-6B and increasing concentrations of 100 nM to 1 uM of SGI-1027. XIST expression was relative to untreated levels, normalized to RPL13a. (C) qPCR results show percentage of CDKL5 wt allele reactivation after 5 days treatment with or without 20 nM ASO-6B and increasing concentrations of 100 nM to 1 uM of SGI-1027. CDKL5 wt reactivation was normalized to CDKL5mut expression from the Xa chromosome.

FIGS. 10A-10C. Xi reactivation in human CDKL5 patient's fibroblast with XIST ASO-6B and DNMT3 inhibitor (Nanaomycin) co-treatment. (A) A human CDKL5 patient fibroblast line carrying mutation on the Xa was treated with an ASO to XIST and a DNMT3i, nanaomycin A. After 5 days of treatment with 20 nM XIST ASO-6B (added once at day 0; lipofectamine transfection) and 25 nM-100 nM nanaomycin A (added every 2 days; days 0, 2, and 4), cells were harvested for qPCR analysis. (B) qPCR results show percentage of XIST expression after 5 days treatment with or without 20 nM ASO-6B and increasing concentrations of nanaomycin A. XIST expression was relative to untreated levels, normalized to RPL13a. (C) qPCR results show percentage of CDKL5 wt allele reactivation after 5 days treatment with or without 20 nM ASO-6B and increasing concentrations of nanaomycin A. CDKL5 wt reactivation was normalized to CDKL5mut expression from the Xa chromosome.

FIGS. 11A-11B. Triplicate experiment showing Xi reactivation in human CDKL5 patient's fibroblast with XIST ASO+/−DNMTi (Decitabine, Azacitidine, RG-108). A human CDKL5 patient fibroblast line carrying mutation on the Xa was treated with ASO to 20 nM XIST ASO 6B (added once at day 0; lipofectamine transfection) with or without one of three DNMTi (1 uM Decitabine, 0.5 uM Azacitidine, 1 uM RG-108 (added every 2 days; days 0 and 2). Cells were harvested at Day 4. (B) qPCR results show percentage of XIST expression after 4 days treatment with or without 20 nM ASO-6B and with or without each of DNMTi (1 uM Decitabine, 0.5 uM Azacitidine, 1 uM RG-108). XIST expression was relative to untreated levels, normalized to RPL13a. (C) qPCR results show percentage of CDKL5 wt allele reactivation after 7 days treatment with or without 20 nM ASO-6B and with or without each of DNMTi (1 uM Decitabine, 0.5 uM Azacitidine, 1 uM RG-108). CDKL5 wt reactivation was normalized to CDKL5mut expression from the Xa chromosome. Data shown are the average of three experiments; error bars are standard deviation.

FIGS. 12A-12B. Xi reactivation with DNMT1 siRNA with and without XIST ASO. A human CDKL5 patient fibroblast line carrying mutation on the Xa was treated with ASO to XIST and siRNAs to DNMT1. On day 0, cells were treated with 20 nM of XIST ASO-1U and/or 25 nM of DNMT siRNA (SEQ ID NOs: 88-91) with and without 1 uM Decitabine or 1 uM RG-108. On day 7, cells were harvested for qPCR analysis. (A) qPCR results show percentage of DNMT1 expression after 7 days treatment with or without 20 nM ASO-1U, and/or 25 nM of DNMT siRNA with and without 1 uM Decitabine or 1 uM RG-108. XIST expression was relative to untreated levels, normalized to RPL13a. (B) qPCR results show percentage of CDKL5 wt allele reactivation after 7 days treatment with or without with or without 20 nM ASO-1U, and/or 25 nM of DNMT siRNA with and without 1 uM Decitabine or 1 uM RG-108. CDKL5 wt reactivation was normalized to CDKL5mut expression from the Xa chromosome.

FIGS. 13A-13D. XIST ASO (ASO-1U), DNMT1 ASO, and DNMTi co-treatment in CDKL5 patient cells. (A) A human CDKL5 patient fibroblast line carrying mutation on the Xa was treated with ASOs to XIST and DNMT. After 5 days of treatment with 20 nM XIST ASO-1U and 20 nM-100 nM DNMT1 ASO (lipofectamine transfection for both, each added once at day 0) with and without 1.0 uM RG-108 (added every 2 days; days 0, 2, and 4), cells were harvested for qPCR analysis. Negative control=HPRT MOE-based ASO. (B) qPCR results show percentage of DNMT1 expression (top bar graph) or XIST expression (bottom bar graph) after 7 days treatment with or without 20 nM XIST ASO-1U, and 20 nM of DNMT1 ASO or 100 nM DNMT1 ASO with and without 1 uM RG-108. DNMT1 expression and XIST expression were relative to lipo only levels, normalized to RPL13a. (C) qPCR results show percentage of DNMT1 expression (top bar graph) or XIST expression (bottom bar graph) after 7 days treatment with or without 20 nM XIST ASO-1U, and 20 nM of DNMT1 ASO with and without 1 uM RG-108. DNMT1 expression and XIST expression were relative to untreated levels, normalized to RPL13a. (D) qPCR results show percentage of CDKL5 wt allele reactivation after 5 days treatment with or without 10 nM ASO-1U and 10 nM DNMT1 ASO (lipofectamine transfection) and with or without 1.0 uM RG-108. CDKL5 wt reactivation was normalized to CDKL5mut expression from the Xa chromosome, normalized to RPL13a.

### DETAILED DESCRIPTION

The Xi is a reservoir of >1000 functional genes that could, in principle, be reactivated, by increasing gene expression, to treat disorders caused by mutations or altered epigenetic regulation on the Xa. In the present study, we set out to define a pharmacological approach for selective Xi-reactivation to restore expression of X-linked gene products. We focused on RTT and restoration of MECP2 gene expression, but our Xi-reactivation platform is agnostic to both the disease and the X-linked gene product. Any gene residing on the X-chromosome could be targeted in phenotypic, heterozygous females.

By targeting factors in the XIST interactome, we found that the anti-sense oligonucleotides (ASOs) targeting XIST RNA described herein, particularly when combined with an epigenetic inhibitor, e.g., a small molecule inhibitor of an XIST interacting protein (e.g., inhibitors of DNA methyltransferase I, RNA methylation, histone modification, or nucleosome remodeling, inter alia) achieved an unprecedented level of Xi-reactivation.

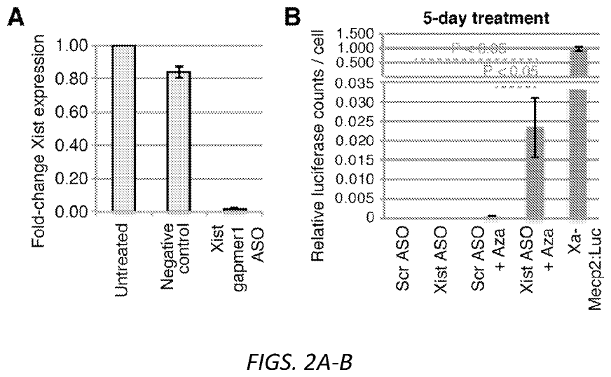

By targeting XIST RNA with an ASO and DNMT1 protein with decitabine (or Aza), we observed a 2 to 5% upregulation—equivalent to a 12,000-30,000x increase in Xi-Mecp2 expression, which is considerably greater than the 600× upregulation observed in a previous screen (Lessing D et al. A high-throughput small molecule screen identifies synergism between DNA methylation and Aurora kinase pathways for X reactivation. Proc Natl Acad Sci USA. 2016; 113:14366-14371), and thus marks significant progress for the Xi-reactivation platform. Several other combinations also yielded a larger degree of reactivation than previously seen with any other compound; these combinations included XIST LNA in combination with EPZ6438 (an EZH2 inhibitor) and XIST LNA in combination with VX680 (an AURK inhibitor). In vivo data have suggested that even 5% of normal Mecp2 levels can have profound impact on survival and overall function, as a previous report showed slightly milder phenotype of the Mecp2-lox-stop-lox male mice, due to their “leaky” termination cassette that enabled read-through Mecp2 transcription (Guy J et al. Reversal of neurological defects in a mouse model of Rett syndrome. Science. 2007; 315:1143-1147). Thus, while the degree of MECP2 gene expression upregulation following treatment with XIST ASO in combination with Aza did not exceed 5% in the experiments disclosed herein, this degree of restoration could have significant phenotypic consequences in vivo. Moreover, because our treatment period was very brief—only 3-5 days—and the tolerable Aza concentrations in cell culture (0.5 μM; FIG. 2C) are still higher than concentrations typically used for mouse IP injections (Sales A J et al. Antidepressant-like effect induced by systemic and intra-hippocampal administration of DNA methylation inhibitors. Br J Pharmacol. 2011; 164:1711-1721), in vivo outcomes may be enhanced by applying a more concentrated dosage in future studies. Our present analysis cannot distinguish between high-level MECP2 reactivation from a few cells versus a low-level reactivation from a large percentage of cells. The two possibilities would have different physiological implications, but both are potentially relevant from a therapeutic standpoint, as MECP2 has been identified to have both cell-autonomous and non-cell-autonomous functions (Kishi and Macklis, (2010) Experimental Neurology 222(1):51-58).

ASO drugs are generally more specific and have the advantage that information on pharmacokinetics and toxicity studies for chemically similar ASOs is transferable and cumulative. Thus, ASOs may have a more favorable path to regulatory approval. Small molecules generally have lower selectivity and may face steeper hurdles in the approval process within the US Food and Drug Administration (FDA). By mixing modalities, our approach may potentially anticipate a more streamlined approach to FDA approval. We also note that Aza has already been FDA-approved for other disease indications (myelodysplastic syndrome and acute myeloid leukemia (Kishi N and Macklis J D. MeCP2 functions largely cell-autonomously, but also non-cell-autonomously, in neuronal maturation and dendritic arborization of cortical pyramidal neurons. Exp Neurol. 2010; 222:51-58). Furthermore, our present in vivo data indicate that Aza need not be given continuously or long term to observe an impact on Xi reactivation in the brain. Nor does Aza need to be injected into the target organ—e.g., the brain. Indeed, three short pulses delivered systemically at the beginning of the treatment period was sufficient to induce Xi reactivation after two weeks in the brain. Unlike LNA-based ASOs which have tissue half-lives of several weeks (Wahlestedt C et al. Potent and nontoxic antisense oligonucleotides containing locked nucleic acids. Proc Natl Acad Sci USA. 2000; 97:5633-5638), Aza is known to have a very short half-life (t_{1/2}<1h in plasma) (Welch J S et al. TP53 and decitabine in acute myeloid leukemia and myelodysplastic syndromes. N Engl J Med. 2016; 375:2023-2036). However, once DNA is demethylated, the state may be stable (Kordasiewicz H B et al. Sustained therapeutic reversal of Huntington's disease by transient repression of huntingtin synthesis. Neuron. 2012; 74:1031-1044). Future work will determine the duration of the effect and whether periodic boosters of either Aza or ASO might be necessary to maintain the reactivation.

Finally, partial Xi-reactivation in the brain does not cause apparent morbidity or mortality in the mouse. When combinations described herein were tested in a Rett-specific disease model that recapitulates the RTT disease severity, phenotypic improvement was seen.

ASOs are well suited for the treatment of neurological diseases and their delivery may be targeted to the central nervous system through intracerebroventricular or intrathecal injection (Southwell A L et al. Antisense oligonucleotide therapeutics for inherited neurodegenerative diseases. Trends Mol Med. 2012; 18:634-643), which has been considered acceptable and safe for serious disease such as ALS (Karahoca M and Momparler R L. Pharmacokinetic and pharmacodynamic analysis of 5-aza-2′-deoxycytidine (decitabine; Aza) in the design of its dose-schedule for cancer therapy. Clin Epigenetics. 2013; 5:3).

### Methods of Reactivating Genes on the Inactive X Chromosome (Xi)

The present disclosure provides methods for reactivating genes on Xi by combining inhibitors for XIST-interacting epigenetic modifying factors (non-limiting list in Table 2). The methods include co-administering an inhibitor of an XIST-interacting epigenetic modifying factor (listed in Table 2), e.g., a small molecule or ASO targeting an epigenetic modifying factor, and a small inhibitory ASO that targets XIST RNA. These methods can be used, e.g., to reactivate genes in single cells, e.g., isolated cells in culture, or in tissues, organs, or whole animals. In some embodiments, the methods are used to reactivate genes on Xi in a cell or subject that has an X-linked disease, e.g., RTT. X-reactivation can be achieved in various cell types, including proliferating fibroblasts and post-mitotic neurons.

The methods described herein can be also be used to specifically re-activate one or more genes on Xi, by co-administering an inhibitory nucleic acid targeting a suppressive RNA or genomic DNA at strong and/or moderate binding sites as described in WO 2012/065143, WO 2012/087983, and WO 2014/025887 or in U.S. Ser. No. 62/010,342 (which are incorporated herein in their entirety), to disrupt RNA-mediated silencing in cis on the inactive X-chromosome. The suppressive RNAs can be noncoding (e.g., long noncoding RNA (lncRNA)) or occasionally part of a coding mRNA; for simplicity, we will refer to them together as suppressive RNAs (supRNAs) henceforth. SupRNAs that mediate silencing of genes on the X chromosome are known in the art; see, e.g., WO 2012/065143, WO 2012/087983, WO 2014/025887 and U.S. Ser. No. 62/010,342, and inhibitory nucleic acids and small molecules targeting (e.g., complementary to) the sRNAs, or complementary or identical to a region within a strong or moderate binding site in the genome, e.g., as described in WO 2014/025887, can be used to modulate gene expression in a cell, e.g., a cancer cell, a stem cell, or other normal cell types for gene or epigenetic therapy. The nucleic acids targeting supRNAs that are used in the methods described herein are termed “inhibitory” (though they increase expression of the supRNA-repressed gene) because they inhibit the supRNAs-mediated repression of a specified gene. Without wishing to be bound to a particular theory, the nucleic acids targeting supRNAs may function either by directly binding to the supRNAs itself (e.g., an antisense oligo that is complementary to the supRNAs) or by binding to a strong or moderate binding site for an RNA-binding protein (e.g., PRC2—also termed an EZH2, SUZ12, and CTCF) in the genome, and in doing so, preventing binding of the RNA-binding protein complex and thus disrupting silencing in the region of the strong or moderate binding site. The inhibitory nucleic acids that bind to a strong or moderate RNA-binding protein binding site can bind to either strand of the DNA, but preferably bind to the same strand to which the supRNAs binds. See, e.g., WO 2012/065143, WO 2012/087983, WO 2014/025887 and U.S. Ser. No. 62/010,342.

The cells can be in vitro, including ex vivo, or in vivo (e.g., in a subject who has cancer, e.g., a tumor).

In some embodiments, the methods include introducing into the cell (or administering to a subject) an inhibitory ASO targeting XIST RNA and an inhibitor of an XIST-interacting protein, e.g., a chromatin-modifying protein, e.g., a small molecule inhibitor of an XIST-interacting protein.

In some embodiments, the methods include introducing into the cell (or administering to a subject) an inhibitory nucleic acid (e.g., targeting XIST RNA) that is modified in some way, e.g., an inhibitory nucleic acid that differs from the endogenous nucleic acids at least by including one or more modifications to the backbone or bases as described herein for inhibitory nucleic acids. Such modified nucleic acids are also within the scope of the present invention.

In some embodiments, the methods include introducing into the cell (or administering to a subject) an inhibitor of XIST RNA (e.g., a small inhibitory RNA (siRNA) or LNA that targets XIST) and an inhibitor of an XIST-interacting protein, e.g., AURKB or EZH2, e.g., a small molecule inhibitor, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342. A nucleic acid that binds “specifically” binds primarily to the target, i.e., to the target DNA, mRNA, or supRNA to inhibit regulatory function or binding of the DNA, mRNA, or supRNA, but does not substantially inhibit function of other non-target nucleic acids. The specificity of the nucleic acid interaction thus refers to its function (e.g., inhibiting gene expression) rather than its hybridization capacity. Inhibitory nucleic acids may exhibit nonspecific binding to other sites in the genome or other RNAs without interfering with binding of other regulatory proteins and without causing degradation of the non-specifically-bound RNA. Thus this nonspecific binding does not significantly affect function of other non-target RNAs and results in no significant adverse effects. These methods can be used to treat an X-linked condition in a subject by administering to the subject a composition or compositions (e.g., as described herein) comprising an inhibitor of XIST RNA and of an XIST-interacting protein, e.g., as listed in Table 2, e.g., a small molecule inhibitor, and optionally an inhibitory nucleic acid that specifically binds, or is complementary, to a strong or moderate binding site or a supRNA (e.g., as described in WO 2012/065143, WO 2012/087983, WO 2014/025887 and U.S. Ser. No. 62/010,342) that is associated with an X-linked disease gene. Examples of genes involved in X-linked diseases are shown in Table 4.

As used herein, treating includes “prophylactic treatment” which means reducing the incidence of or preventing (or reducing risk of) a sign or symptom of a disease in a patient at risk for the disease, and “therapeutic treatment”, which means reducing signs or symptoms of a disease, reducing progression of a disease, reducing severity of a disease, in a patient diagnosed with the disease.

In some embodiments, the methods described herein include administering a composition, e.g., a sterile composition, comprising an inhibitory nucleic acid that is complementary to XIST or a gene encoding XIST RNA, e.g., as listed in Table 1, and an inhibitor of an XIST-interacting protein, e.g., as listed in Table 2, and optionally an inhibitory nucleic acid that is complementary to a supRNA as known in the art, e.g., as described in WO 2012/065143, WO 2012/087983, and/or WO 2014/025887. Inhibitory nucleic acids for use in practicing the methods described herein can be an antisense or small interfering RNA, including but not limited to an shRNA or siRNA. In some embodiments, the inhibitory nucleic acid is a modified nucleic acid polymer (e.g., a locked nucleic acid (LNA) molecule).

Inhibitory nucleic acids have been employed as therapeutic moieties in the treatment of disease states in animals, including humans. Inhibitory nucleic acids can be useful therapeutic modalities that can be configured to be useful in treatment regimens for the treatment of cells, tissues and animals, especially humans.

For therapeutics, an animal, preferably a human, who has an X-linked disorder is treated by administering an XIST ASO and an inhibitor of an XIST-interacting protein, e.g., as listed in Table 2, e.g., a small molecule inhibitor.

### Inhibitor of XIST RNA

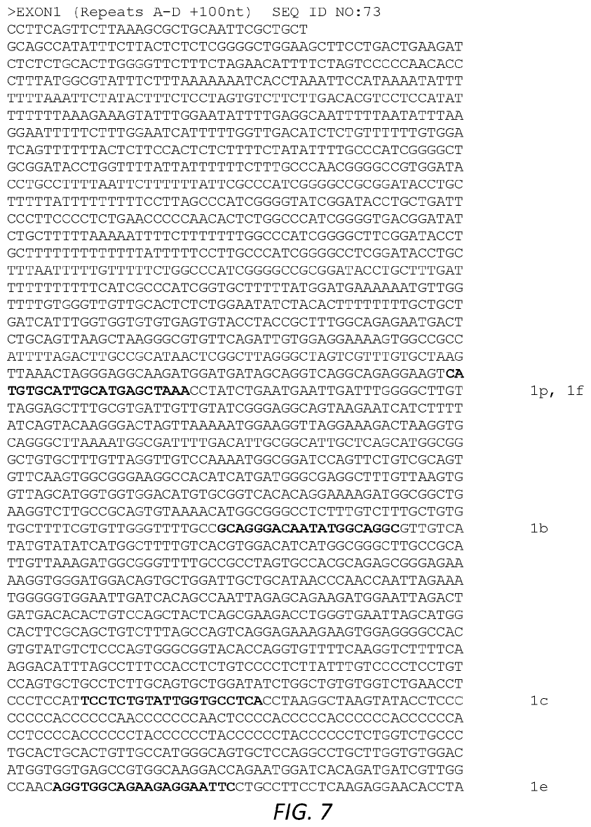

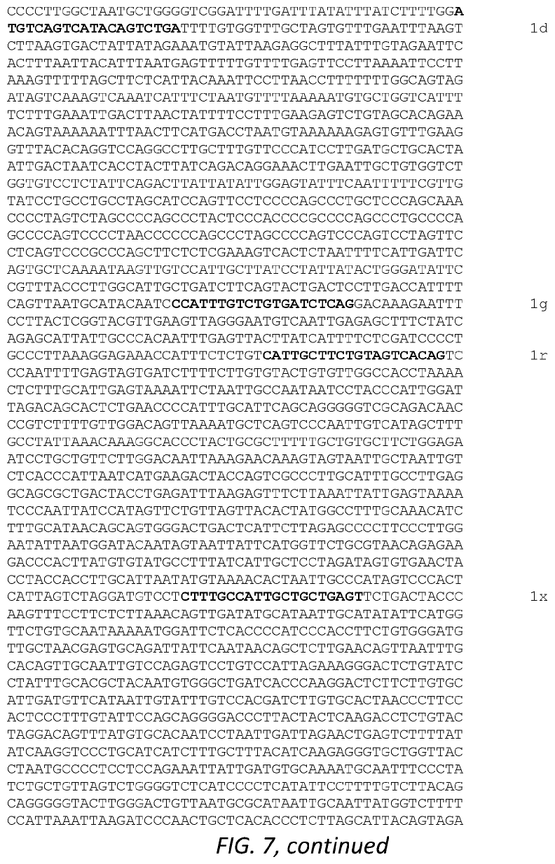

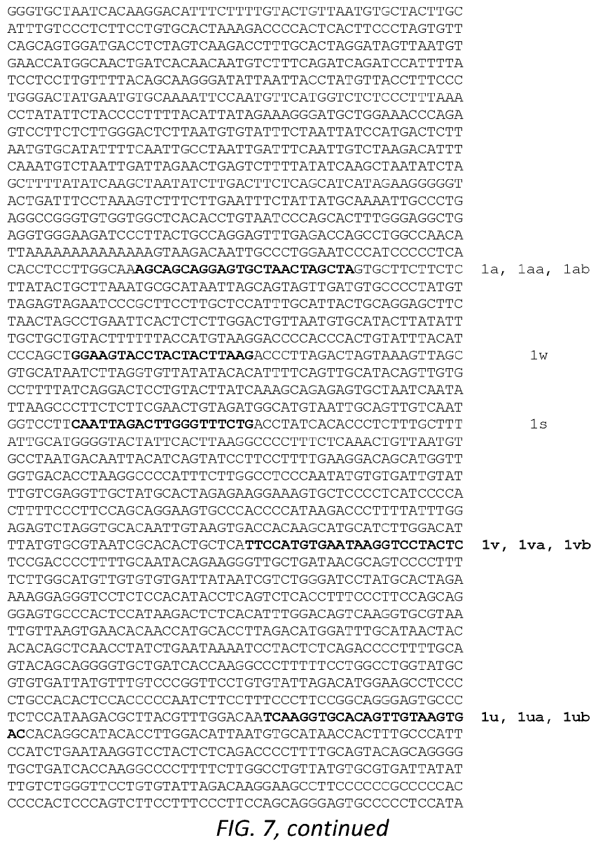

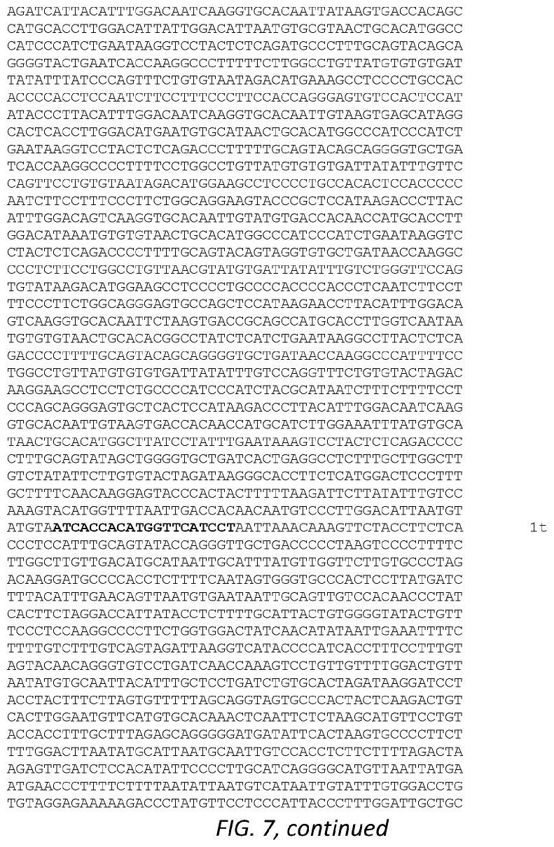

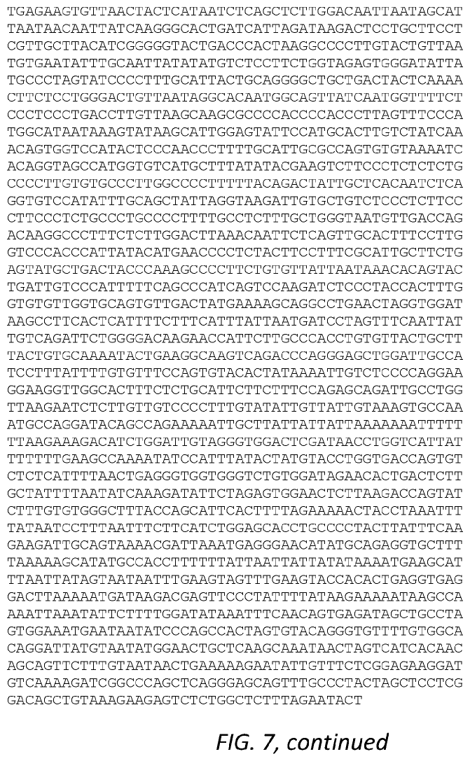

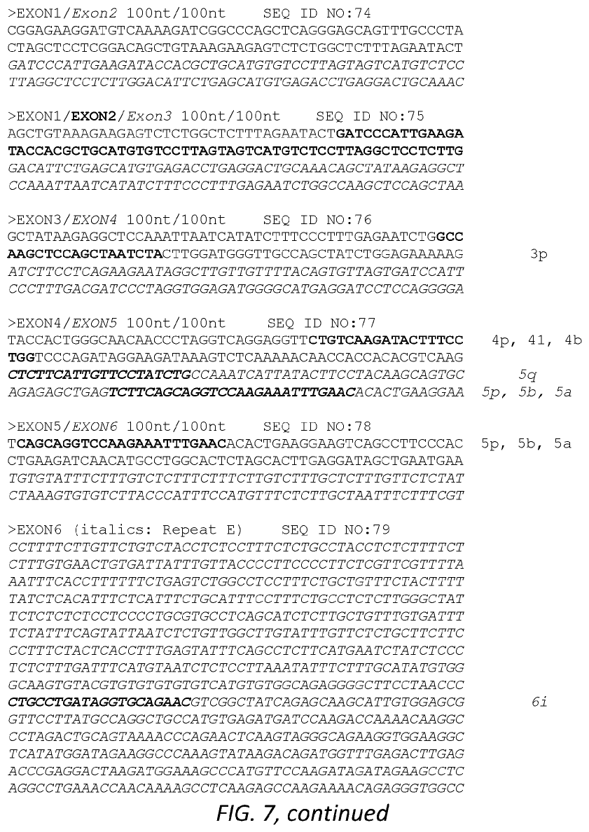

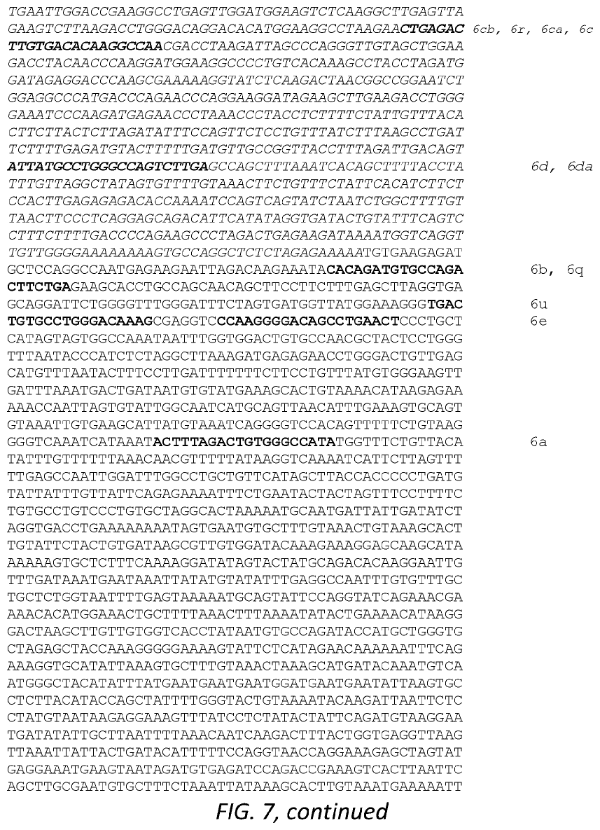

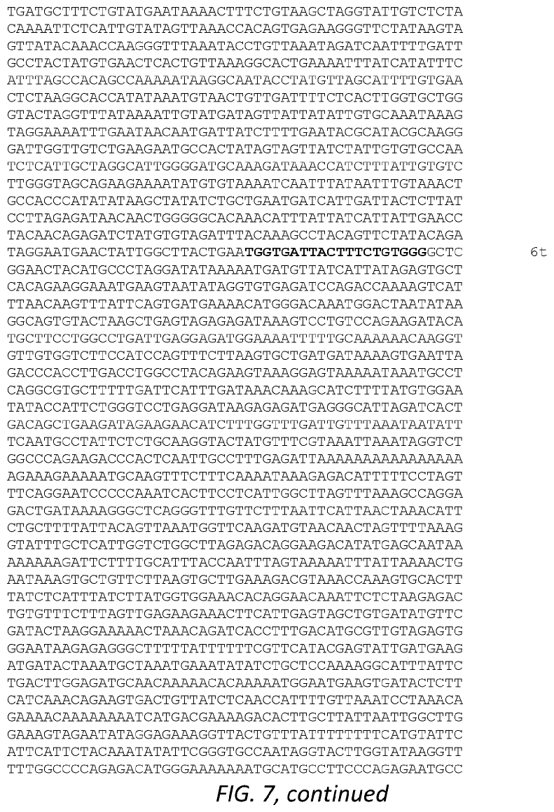

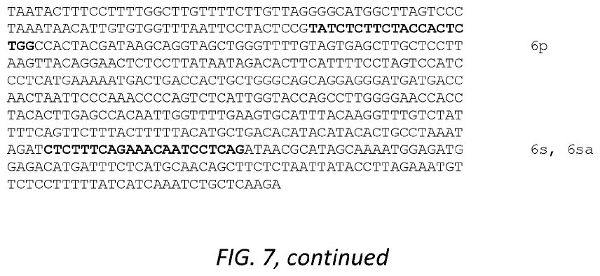

The methods include administering an inhibitor of an XIST RNA itself, e.g., an inhibitory nucleic acid targeting XIST RNA. Although in typical usage XIST refers to the human sequence and XIST to the mouse sequence, in the present application the terms are used interchangeably. The human XIST sequence is available in the ensemble database at ENSG00000229807; it is present on Chromosome X at 73,820,651-73,852,753 reverse strand (Human GRCh38.p2). The sequences of exons 1-6 are shown in SEQ ID NOs:73-79 (see FIG. 7); XIST exons correspond to 601-11972 (exon 1); 15851-15914 (exon 2); 19593-20116 (exon 3); 21957-21984 (exon 4); 22080-22288 (exon 5); and 23887-33304 (exon 6). Alternatively, see NCBI Reference Sequence: NR 001564.2, Homo sapiens X inactive specific transcript (non-protein coding) (XIST), long non-coding RNA, wherein the exons correspond to 1-11372, 11373-11436, 11437-11573, 11574-11782, 11783-11946, and 11947-19280. The inhibitory nucleic acid targeting XIST RNA can be any inhibitory nucleic acid as described herein, and can include modifications described herein or known in the art. In some embodiments, the inhibitory nucleic acid is an antisense oligonucleotide (ASO) that targets a sequence in XIST RNA, e.g., a sequence within an XIST exon as shown in SEQ ID NO:1-45 or within the RNA sequence as set forth in NR 001564.2, preferably wherein the ASO comprises a sequence as shown in Table 1. In some embodiments, the inhibitory nucleic includes at least one locked nucleotide, e.g., is a locked nucleic acid (LNA). Table 1 provides a list of ASOs that have more than 80% XIST knock down when cells are treated for 72 hrs with the below XIST ASOs.

TABLE 1
XIST ANTISENSE OLIGONUCLEOTIDE SEQUENCES

| | | Starting | | | | |
|---|---|---|---|---|---|---|
| | | nt no. | | | | |
| | | in human | | | | ASO |
| | | XIST | | | | conc |
| | | XIST | | | % | at |
| | ASO | tran- | ASO sequence | | Max | max |
| No. | ID | script | 5′->3′ | SI# | KD | KD |
| 1 | 6A | 73045510 | TATGGCCCAC | 1. | 99.3 | 20 |
| | | | AGTCTAAAGT | | | |
| 2 | 1P | 73071538 | TAGCTCATGC | 2. | 99 | 100 |
| | | | AATGCACATG | | | |
| 3 | 1W | 73066929 | CTTAAGTAGT | 3. | 99 | 20 |
| | | | AGGTACTTCC | | | |
| 4 | 3P | 73053106 | TAGATTAGCT | 4. | 99 | 100 |
| | | | GGAGCTTGGC | | | |
| 5 | 6Q | 73045989 | TCAGAAGTCT | 5. | 99 | 100 |
| | | | GGCACATCTG | | | |
| 6 | 6R | 73046681 | GGCCTTGTGT | 6. | 99 | 100 |
| | | | CACAAGTCTC | | | |
| 7 | 1A | 73067172 | TAGTTAGCAC | 7. | 98 | 20 |
| | | | TCCTGCTGCT | | | |
| 8 | 1D | 73070287 | TCAGACTGTA | 8. | 98 | 20 |
| | | | TGACTGACAT | | | |
| 9 | 1F | 73071536 | TTTAGCTCAT | 9. | 98 | 20 |
| | | | GCAATGCACA | | | |
| 10 | 1V | 73066360 | AGGACCTTAT | 10. | 98 | 20 |
| | | | TCACATGGAA | | | |
| 11 | 6B | 73045991 | AGAAGTCTGG | 11. | 98 | 100 |
| | | | CACATCTGTG | | | |
| 12 | 6C | 73046679 | TTGGCCTTGT | 12. | 97 | 20 |
| | | | GTCACAAGTC | | | |
| 13 | 6U | 73045880 | CTTTGTCCCA | 13. | 97 | 20 |
| | | | GGCACAGTCA | | | |
| 14 | 1B | 73071113 | GCCTGCCATA | 14. | 96 | 20 |
| | | | TTGTCCCTGC | | | |
| 15 | 1U | 73065858 | CTTACAACTG | 15. | 96 | 100 |
| | | | TGCACCTTGA | | | |
| 16 | 5A | 73048982 | CAAATTTCTT | 16. | 96 | 20 |
| | | | GGACCTGCTG | | | |
| 17 | 6D | 73046324 | TCAAGACTGG | 17. | 96 | 20 |
| | | | CCCAGGCATA | | | |
| 18 | 1C | 73070628 | TGAGGCACCA | 18. | 95 | 20 |
| | | | ATACAGAGGA | | | |
| 19 | 1G | 73069468 | CTGAGATCAC | 19. | 95 | 20 |
| | | | AGACAAATGG | | | |
| 20 | 5B | 73048986 | TTTCTTGGAC | 20. | 95 | 20 |
| | | | CTGCTGAAGA | | | |
| 21 | 6S | 73041820 | CTGAGGATTG | 21. | 95 | 100 |
| | | | TTTCTGAAAG | | | |
| 22 | 1R | 73069308 | CTGTGACTAC | 22. | 94 | 100 |
| | | | AGAAGCAATG | | | |
| 23 | 1S | 73066729 | CAGAAACCCA | 23. | 94 | 20 |
| | | | AGTCTAATTG | | | |
| 24 | 1T | 73064081 | AGGATGAACC | 24. | 93 | 20 |
| | | | ATGTGGTGAT | | | |
| 25 | 6I | 73047076 | GTTCTGCACC | 25. | 93 | 20 |
| | | | TATCAGGCAG | | | |
| 26 | 6E | 73045853 | AGTTCAGGCT | 26. | 92 | 20 |
| | | | GTCCCCTTGG | | | |
| 27 | 6P | 73042143 | CCAGAGTGGT | 27. | 92 | 20 |
| | | | AGAAGAGATA | | | |
| 28 | 1E | 73070381 | GAATTCCTCT | 28. | 91 | 20 |
| | | | TCTGCCACCT | | | |
| 29 | 1X | 73068617 | ACTCAGCAGC | 29. | 89 | 100 |
| | | | AATGGCAAAG | | | |
| 30 | 5Q | 73049047 | CAGATAGGAA | 30. | 89 | 100 |
| | | | CAATGAAGAG | | | |
| 31 | 6T | 73043750 | CCCACAGAAA | 31. | 89 | 20 |
| | | | GTAATCACCA | | | |
| 32 | 4B | 73050948 | CCAGGAAAGT | 32. | 88 | 100 |
| | | | ATCTTGACAG | | | |
| 33 | 4P | 73050984 | GTTGTTGCCC | 33. | 88 | 100 |
| | | | AGTGGTAGTG | | | |
| 34 | 5P | 73048979 | GTTCAAATTT | 34. | 88 | 20 |
| | | | CTTGGACCTG | | | |
| 35 | 4A | 73050983 | GGTTGTTGCC | 35. | 84 | 20 |
| | | | CAGTGGTAGT | | | |

SI#, SEQ ID NO:

TABLE 1
XIST ANTISENSE OLIGONUCLEOTIDE SEQUENCES

| | | SEQ ID |
|---|---|---|
| ASO ID | SEQUENCE | NO: |
| 1a | TAGTTAGCACTCCTGCTGCT | 7 |
| 1aa | GCTAGTTAGCACTCCTGCTG | 36 |
| 1ab | TAGCTAGTTAGCACTCCTGC | 37 |
| 1u | CTTACAACTGTGCACCTTGA | 15 |
| 1ua | CACTTACAACTGTGCACCTT | 38 |
| 1ub | GTCACTTACAACTGTGCACC | 39 |
| 1v | AGGACCTTATTCACATGGAA | 10 |
| 1va | GTAGGACCTTATTCACATGG | 40 |
| 1vb | GAGTAGGACCTTATTCACAT | 41 |
| 6c | TTGGCCTTGTGTCACAAGTC | 12 |
| 6ca | GGCCTTGTGTCACAAGTCTC | 42 |
| 6cb | CCTTGTGTCACAAGTCTCAG | 43 |
| 6d | TCAAGACTGGCCCAGGCATA | 17 |
| 6da | AAGACTGGCCCAGGCATAAT | 44 |
| 6s | CTGAGGATTGTTTCTGAAAG | 21 |
| 6sa | GAGGATTGTTTCTGAAAGAG | 45 |

### XIST-Interacting Proteins

The methods include administering an inhibitor of an XIST-interacting protein. Tables 5 and 6 of PCT/US2016/026218 (published as WO2016164463, which is incorporated by reference here in its entirety), and Table 2 herein, list XIST-interacting proteins, e.g., chromatin-modifying proteins, that can be targeted in the methods described herein. These inhibitors can include small molecules as well as inhibitory nucleic acids targeting the XIST-interacting protein.

Small molecule inhibitors of many of these XIST interactors are known in the art; see, e.g., Table 2, for examples. In addition, small molecule inhibitors of PRC1 or PRC2 components can be used; for example, inhibitors of EZH2 include UNC1999, E7438, N-[(4,6-dimethyl-2-oxo-1,2-dihydro-3-pyridinyl)methyl]-3-methyl-1-[(1S)-1-methylpropyl]-6-[6-(1-piperazinyl)-3-pyridinyl]-1H-indole-4-carboxamide, EPZ-6438 (N-((4,6-dimethyl-2-oxo-1,2-dihydropyridin-3-yl)methyl)-5-(ethyl(tetrahyd-ro-2H-pyran-4-yl)amino)-4-methyl-4′-(morpholinomethyl)-[1,1′-biphenyl]-3-c-arboxamide), GSK-126 ((S)-1-(sec-butyl)-N-(4,6-dimethyl-2-oxo-1,2-dihydropyridin-3-yl)methyl)-3-methyl-6-(6-(piperazin-1-yl)pyridin-3-yl)-1H-indole-4-carboxamide), GSK-343 (1-Isopropyl-N-((6-methyl-2-oxo-4-propyl-1,2-dihydropyridin-3-yl)-methyl)-6-(2-(4-methylpiperazin-1-yl)pyridine-4-yl)-1H-indazole-4-carboxam-ide), E11, 3-deazaneplanocin A (DNNep, 5R-(4-amino-1H-imidazo[4,5-c]pyridin-1-yl)-3-(hydroxymethyl)-3-cyclopente-ne-1S,2R-diol), isoliquiritigenin, and those provided in, for example, U.S. Publication Nos. 2009/0012031, 2009/0203010, 2010/0222420, 2011/0251216, 2011/0286990, 2012/0014962, 2012/0071418, 2013/0040906, US20140378470, US20140275081, US20140357688, and 2013/0195843; see also PCT/US2011/035336, PCT/US2011/035340, PCT/US2011/035344.

TABLE 2
Exemplary Xist-Interacting Proteins
and Chromatin-Modifying Proteins

| Name of Xist- | |
|---|---|
| Interacting Protein | Small molecule inhibitor |
| WAPL | — |
| SMC1a | See above |
| SMC3 | See above |
| RAD21 | See above |
| KIF4 | — |
| PDS5a/b | See above |
| CTCF | 3-aminobenzamide |
| TOP1 | See above |
| TOP2a | See above |
| TOP2b | See above |
| SMARCA4 (BRG1) | PFI3 ((E)-1-(2-Hydroxyphenyl)-3-((1R,4R)-5- |
| | (pyridin-2-yl)-2,5-diazabicyclo[2.2.1]heptan-2- |
| | yl)prop-2-en-1-one); JQ1(+); AGN-PC-0DAUWN |
| SMARCA5 | — |
| SMARCC1 | — |
| SMARCC2 | — |
| SMARCB1 | — |
| CBX2 | — |
| CBX4 | — |
| CBX5 | — |
| CBX6 | — |
| CBX7 | MS37452 |
| CBX8 | — |
| RINB1a | PRT4165 (2-pyridine-3-yl-methylene-indan- |
| | 1,3-dione) |
| RING1b | — |
| AURKB | ZM447439, Hesperadin, VX-680/MK-0457 (4,6- |
| | diaminopyrimidine), AT9283, AZD1152, |
| | AKI-001, PHA-680632, VE-465, JNJ-7706621, |
| | CCT129202, MLN8237, ENMD-2076, MK-5108, |
| | PHA-739358, CYC116, SNS-314, R763, |
| | PF-03814375, GSK1070916, AMG-900 (see |
| | Kollareddy et al., Invest New Drugs. 2012 |
| | December; 30(6): 2411-2432) |
| SPEN/MINT/SHARP | MG132 |
| DNMT1/DNMT3a/3b | See below, e.g., WO2016164463, |
| | WO2014039860 |
| SmcHD1 | — |
| CTCF | — |
| MYEF2 | — |
| ELAVL1 | — |
| SUN2 | mevinolin |
| Lamin-B Receptor | — |
| (LBR) | |
| LAP | bestatin |
| hnRPU/SAF-A | - DPQ |
| hnRPK | — |
| hnRPC | — |
| PTBP2 | — |
| RALY | — |
| MATRIN3 | plumbagin |
| MacroH2A | |
| ATRX | Berberine, Inhibitors of histone deacetylases |
| | (HDAC) such as trichostatin A (TSA), |
| | depsipeptide, vorinostat, |
| RYBP | — |
| YY1 | — |
| EZH2 | See above |
| SUZ12 | — |
| EED | Astemizole (inhibits EZH2-EED interaction) |
| RBBP7 | — |
| RBBP4 | — |
| JARID2 | — |

DNA methyltransferase (DNMT) Inhibitors

A number of DNMT inhibitors (against DNMT1, DNMT2, DNMT3a/b, as several examples) are known in the art, including 5-azacytidine (azacytidine, Azacitidine, 4-amino-1-beta-D-ribofuranosyl-s-triazin-2(1H)-one, Vidaza), decitabine (5-aza-2′-deoxycytidine, Dacogen), Zebularine (pyrimidin-2-one beta-ribofuranoside), procainamide, procaine, psammaplin A, sinefungin, temozolomide, OM173-alphaA, DNMT3A-binding protein, theaflavin 3,3′-digallate, 1-Hydrazinophthalazine, SGI-1027 (N-[4-[(2-Amino-6-methyl-4-pyrimidinyl)amino]phenyl]-4-(4-quinolinylamino)benzamide), hydralazine, NSC14778, Olsalazine, Nanaomycin, SID 49645275, Δ²-isoxazoline, epigallocatechin-3-gallate (EGCG), MG98, SGI-110 (2′-deoxy-5-azacytidylyl-(3′→5′)-2′-deoxyguanosine), RG-108 (N-phthalyl-L-tryptophan), SW155246, SW15524601, SW155246-2, and DZNep (SGI-1036, 3-deazaneplanocin A). See also Medina-Franco et al., Int. J. Mol. Sci. 2014, 15(2), 3253-3261; Yoo et al., Computations Molecular Bioscience, 1(1):7-16 (2011); WO2016164463; WO2014039860; etc.

### Topoisomerase Inhibitors

A number of topoisomerase inhibitors (against TOP1, TOP2a/b, as examples) are known in the art; in some embodiments, the topoisomerase inhibitor is an inhibitor of topoisomerase II. Exemplary inhibitors of topoisomerase I include camptothecin and its derivatives such as topotecan, irinotecan, lurtotecan, exatecan, diflometecan, S39625, CPT 11, SN38, gimatecan and belotecan; stibogluconate; indenoisoquinolines (e.g., 2,3-dimethoxy-12h-[1,3]dioxolo[5,6]indeno[1,2-c]isoquinolin-6-ium and 4-(5,11-dioxo-5h-indeno[1,2-c]isoquinolin-6(11h)-yl)butanoate) and indolocarbazoles. See, e.g., Pommier, Chem Rev. 2009 July; 109(7): 2894-2902; Pommier, Nat Rev Cancer. 2006 October; 6(10):789-802.; Sheng et al., Curr Med Chem. 2011; 18(28):4389-409. Exemplary inhibitors of topoisomerase II include etoposide, teniposide, mitoxantrone, amsacrine, saintopin, ICRF-193, genistein, CP-115,953, ellipticine, banoxantrone, Celastrol, N U 2058, Dexrazoxane, and anthracyclines (e.g., doxorubicin, daunorubicin, epirubicin, and idarubicin). See, e.g., Froelich-Ammon and Osheroff, Journal of Biological Chemistry, 270:21429-21432 (1995); Hande, Update on Cancer Therapeutics 3:13-26 (2008).

### Inhibitory Nucleic Acids Targeting XIST-Interacting Proteins

The methods and compositions described herein can include nucleic acids to such as a small inhibitory RNA (siRNA) or LNA that targets (specifically binds, or is complementary to) an RNA or a gene encoding an XIST-interacting protein, e.g., a chromatin-modifying protein (e.g., DNMT), and optionally an inhibitory nucleic acid that targets a strong or moderate binding site or a supRNA described in WO 2012/065143, WO 2012/087983, WO 2014/025887 and U.S. Ser. No. 62/010,342. Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, molecules comprising modified bases, locked nucleic acid (LNA) molecules, bridged nucleic acid (BNA) molecules, peptide nucleic acid (PNA) molecules, and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., U.S. Ser. No. 62/010,342, WO 2012/065143, WO 2012/087983, WO 2014/025887, US 20070117767 A1, U.S. Pat. Nos. 8,987,220, 8,513,401, WO 2016/112374, and US 20150376612 A1. However, in some embodiments the inhibitory nucleic acid is not an miRNA, an stRNA, an shRNA, an siRNA, an RNAi, or a dsRNA.

TABLE 3

| | | Human | | |
|---|---|---|---|---|
| Human | Human | Gene | Gene | |
| Gene ID | Protein | symbol | Synonyms | Accession numbers |
| 2146 | EZH2 | EZH2 | KMT6; KMT6A; | XM_011515896; XM_011515897; |
| | | | WVS; EZH2b; | XM_011515901; NM_001203249; |
| | | | ENX-1; EZH1; | XM_005249964; XM_011515884; |
| | | | ENX1; WVS2 | XM_011515890; XM_011515894; |
| | | | | XM_011515899; NM_004456; |
| | | | | XM_011515886; XM_011515892; |
| | | | | XM_011515900; NM_152998; |
| | | | | XM_011515888; XM_011515889; |
| | | | | XM_011515902; ; NM_001203247; |
| | | | | NM_001203248; XM_005249962; |
| | | | | XM_011515895; XM_011515883; |
| | | | | XM_005249963; XM_011515885; |
| | | | | XM_011515887; XM_011515898; |
| | | | | XM_011515891; XM_011515893 |
| 9212 | AURKB | AURKB | STK5; aurkb-sv2; | XM_011524070; XR_934118; |
| | | | AurB; ARK2; | NM_001256834; XM_011524071; |
| | | | PPP1R48; | XR_934117; NM_001284526; |
| | | | aurkb-sv1; | NM_004217; XM_011524072 |
| | | | AIM-1; AIK2; | |
| | | | IPL1; AIM1; | |
| | | | STK12; STK-1; | |
| | | | ARK-2 | |

### Inhibitory Nucleic Acids

In some embodiments, the inhibitory nucleic acids used in the present methods and compositions are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity between inhibitory nucleic acid and target is not required for the inhibitory nucleic acid to to sufficiently inhibit function of the target.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 for antisense oligos; US2010/0249052 for double-stranded ribonucleic acid (dsRNA); US2009/0181914 and US2010/0234451 for LNAs; US2007/0191294 for siRNA analogues; US2008/0249039 for modified siRNA; and WO2010/129746 and WO2010/040112 for inhibitory nucleic acids, as well as WO2012/065143, WO 2012/087983, and WO 2014/025887 for inhibitory nucleic acids targeting non-coding RNAs/supRNAs; all of which are incorporated herein by reference in their entirety.

### Antisense

In some embodiments, the inhibitory nucleic acids are antisense oligonucleotides (ASOs). ASOs are typically designed to block expression of a DNA or RNA target by binding to the target and halting expression at the level of transcription, translation, or splicing. ASOs of the present invention are complementary nucleic acid sequences designed to hybridize under stringent conditions to an RNA. Thus, oligonucleotides are chosen that are sufficiently complementary to the target, i.e., that hybridize sufficiently well and with sufficient specificity, to confer the desired effect.

siRNA/shRNA

In some embodiments, the nucleic acid sequence that is complementary to an target RNA can be an interfering RNA, including but not limited to a small interfering RNA (“siRNA”) or a small hairpin RNA (“shRNA”). Methods for constructing interfering RNAs are well known in the art. For example, the interfering RNA can be assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure); the antisense strand comprises nucleotide sequence that is complementary to a nucleotide sequence in a target nucleic acid molecule or a portion thereof (i.e., an undesired gene) and the sense strand comprises nucleotide sequence corresponding to the target nucleic acid sequence or a portion thereof. Alternatively, interfering RNA is assembled from a single oligonucleotide, where the self-complementary sense and antisense regions are linked by means of nucleic acid based or non-nucleic acid-based linker(s). The interfering RNA can be a polynucleotide with a duplex, asymmetric duplex, hairpin or asymmetric hairpin secondary structure, having self-complementary sense and antisense regions, wherein the antisense region comprises a nucleotide sequence that is complementary to nucleotide sequence in a separate target nucleic acid molecule or a portion thereof and the sense region having nucleotide sequence corresponding to the target nucleic acid sequence or a portion thereof. The interfering can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNA interference.

In some embodiments, the interfering RNA coding region encodes a self-complementary RNA molecule having a sense region, an antisense region and a loop region. Such an RNA molecule when expressed desirably forms a “hairpin” structure, and is referred to herein as an “shRNA.” The loop region is generally between about 2 and about 10 nucleotides in length. In some embodiments, the loop region is from about 6 to about 9 nucleotides in length. In some embodiments, the sense region and the antisense region are between about 15 and about 20 nucleotides in length. Following post-transcriptional processing, the small hairpin RNA is converted into a siRNA by a cleavage event mediated by the enzyme Dicer, which is a member of the RNase III family. The siRNA is then capable of inhibiting the expression of a gene with which it shares homology. For details, see Brummelkamp et al., Science 296:550-553, (2002); Lee et al, Nature Biotechnol., 20, 500-505, (2002); Miyagishi and Taira, Nature Biotechnol 20:497-500, (2002); Paddison et al. Genes & Dev. 16:948-958, (2002); Paul, Nature Biotechnol, 20, 505-508, (2002); Sui, Proc. Natl. Acad. Sd. USA, 99(6), 5515-5520, (2002); Yu et al. Proc NatlAcadSci USA 99:6047-6052, (2002); US 20070117767 A1, U.S. Pat. Nos. 8,987,220, 8,513,401, WO 2016/112374, and US 20150376612 A1.

The target RNA cleavage reaction guided by siRNAs is highly sequence specific. In general, siRNA containing a nucleotide sequences identical to a portion of the target nucleic acid are preferred for inhibition. However, 100% sequence identity between the siRNA and the target gene is not required to practice the present invention. Thus the invention has the advantage of being able to tolerate sequence variations that might be expected due to genetic mutation, strain polymorphism, or evolutionary divergence. For example, siRNA sequences with insertions, deletions, and single point mutations relative to the target sequence have also been found to be effective for inhibition. Alternatively, siRNA sequences with nucleotide analog substitutions or insertions can be effective for inhibition. In general the siRNAs must retain specificity for their target, i.e., must not directly bind to, or directly significantly affect expression levels of, transcripts other than the intended target.

### Ribozymes

Trans-cleaving enzymatic nucleic acid molecules can also be used; they have shown promise as therapeutic agents for human disease (Usman & McSwiggen, 1995 Ann. Rep. Med. Chem. 30, 285-294; Christoffersen and Marr, 1995 J. Med. Chem. 38, 2023-2037). Enzymatic nucleic acid molecules can be designed to cleave specific RNA targets within the background of cellular RNA. Such a cleavage event renders the RNA non-functional.

In general, enzymatic nucleic acids with RNA cleaving activity act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

Several approaches such as in vitro selection (evolution) strategies (Orgel, 1979, Proc. R. Soc. London, B 205, 435) have been used to evolve new nucleic acid catalysts capable of catalyzing a variety of reactions, such as cleavage and ligation of phosphodiester linkages and amide linkages, (Joyce, 1989, Gene, 82, 83-87; Beaudry et al., 1992, Science 257, 635-641; Joyce, 1992, Scientific American 267, 90-97; Breaker et al, 1994, TIBTECH 12, 268; Bartel et al, 1993, Science 261:1411-1418; Szostak, 1993, TIBS 17, 89-93; Kumar et al, 1995, FASEB J., 9, 1183; Breaker, 1996, Curr. Op. Biotech., 1, 442). The development of ribozymes that are optimal for catalytic activity would contribute significantly to any strategy that employs RNA-cleaving ribozymes for the purpose of regulating gene expression. The hammerhead ribozyme, for example, functions with a catalytic rate (kcat) of about 1 min⁻¹ in the presence of saturating (10 rnM) concentrations of Mg²⁺ cofactor. An artificial “RNA ligase” ribozyme has been shown to catalyze the corresponding self-modification reaction with a rate of about 100 min⁻¹. In addition, it is known that certain modified hammerhead ribozymes that have substrate binding arms made of DNA catalyze RNA cleavage with multiple turn-over rates that approach 100 min⁻¹.

### Modified Inhibitory Nucleic Acids

In some embodiments, the inhibitory nucleic acids used in the methods described herein are modified, e.g., comprise one or more modified bonds or bases. A number of modified bases include phosphorothioate, methylphosphonate, peptide nucleic acids, or locked nucleic acid (LNA) molecules. Some inhibitory nucleic acids are fully modified, while others are chimeric and contain two or more chemically distinct regions, each made up of at least one nucleotide. These inhibitory nucleic acids typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric inhibitory nucleic acids of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the inhibitory nucleic acid comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (i.e., higher target binding affinity) than; 2′-deoxyoligonucleotides against a given target.

A number of nucleotide and nucleoside modifications have been shown to make the inhibitory nucleic acid into which they are incorporated more resistant to nuclease digestion than the native oligodeoxynucleotide; these modified oligos survive intact for a longer time than unmodified inhibitory nucleic acids. Specific examples of modified inhibitory nucleic acids include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are inhibitory nucleic acids with phosphorothioate backbones and those with heteroatom backbones, particularly CH2-NH—O—CH2, CH,˜N(CH3)˜O˜CH2 (known as a methylene(methylimino) or MMI backbone], CH2-O—N(CH3)-CH2, CH2-N(CH3)-N(CH3)-CH2 and O—N(CH3)-CH2-CH2 backbones, wherein the native phosphodiester backbone is represented as O— P— O— CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the inhibitory nucleic acid is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991.

Cyclohexenyl nucleic acid inhibitory nucleic acid mimetics are described in Wang et al., J. Am. Chem. Soc., 2000, 122, 8595-8602.

Modified inhibitory nucleic acid backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

One or more substituted sugar moieties can also be included, e.g., one of the following at the 2′ position: OH, SH, SCH₃, F, OCN, OCH₃ OCH₃, OCH₃ O(CH₂)n CH₃, O(CH₂)n NH₂ or O(CH₂)n CH₃ where n is from 1 to about 10; Ci to C10 lower alkyl, alkoxyalkoxy, substituted lower alkyl, alkaryl or aralkyl; Cl; Br; CN; CF3; OCF3; O-, S-, or N-alkyl; O—, S-, or N-alkenyl; SOCH3; SO2 CH3; ONO2, NO2; N3; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an inhibitory nucleic acid; or a group for improving the pharmacodynamic properties of an inhibitory nucleic acid and other substituents having similar properties. A preferred modification includes 2′-methoxyethoxy [2′-O—CH₂CH₂OCH₃, also known as 2′-O-(2-methoxyethyl)] (Martin et al, HeIv. Chim. Acta, 1995, 78, 486). Other preferred modifications include 2′-methoxy (2′-O—CH₃), 2′-propoxy (2′-OCH₂ CH₂CH₃) and 2′-fluoro (2′-F). Similar modifications may also be made at other positions on the inhibitory nucleic acid, particularly the 3′ position of the sugar on the 3′ terminal nucleotide and the 5′ position of 5′ terminal nucleotide. Inhibitory nucleic acids may also have sugar mimetics such as cyclobutyls in place of the pentofuranosyl group.

Inhibitory nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 8-azaguanine, 7-deazaguanine, N6 (6-aminohexyl)adenine and 2,6-diaminopurine. Kornberg, A., DNA Replication, W. H. Freeman & Co., San Francisco, 1980, pp 75-77; Gebeyehu, G., et al. Nucl. Acids Res. 1987, 15:4513). A “universal” base known in the art, e.g., inosine, can also be included. 5-Me-C substitutions have been shown to increase nucleic acid duplex stability by 0.6-1.2<0>C. (Sanghvi, Y. S., in Crooke, S. T. and Lebleu, B., eds., Antisense Research and Applications, CRC Press, Boca Raton, 1993, pp. 276-278) and are presently preferred base substitutions.

It is not necessary for all positions in a given inhibitory nucleic acid to be uniformly modified, and in fact more than one of the aforementioned modifications may be incorporated in a single inhibitory nucleic acid or even at within a single nucleoside within an inhibitory nucleic acid.

In some embodiments, both a sugar and an internucleoside linkage, i.e., the backbone, of the nucleotide units are replaced with novel groups. The base units are maintained for hybridization with an appropriate nucleic acid target compound. One such oligomeric compound, an inhibitory nucleic acid mimetic that has been shown to have excellent hybridization properties, is referred to as a peptide nucleic acid (PNA). In PNA compounds, the sugar-backbone of an inhibitory nucleic acid is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds comprise, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Inhibitory nucleic acids can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, to 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in ‘The Concise Encyclopedia of Polymer Science And Engineering’, pages 858-859, Kroschwitz, J. I., ed. John Wiley & Sons, 1990, those disclosed by Englisch et al., Angewandle Chemie, International Edition', 1991, 30, page 613, and those disclosed by Sanghvi, Y. S., Chapter 15, Antisense Research and Applications', pages 289-302, Crooke, S. T. and Lebleu, B. ea., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, Y. S., Crooke, S. T. and Lebleu, B., eds, ‘Antisense Research and Applications’, CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the inhibitory nucleic acids are chemically linked to one or more moieties or conjugates that enhance the activity, cellular distribution, or cellular uptake of the inhibitory nucleic acid. Such moieties comprise but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmacokinetic properties, in the context of this invention, include groups that improve uptake, distribution, metabolism or excretion of the compounds of the present invention. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

### Locked Nucleic Acids (LNAs)

In some embodiments, the modified inhibitory nucleic acids used in the methods described herein comprise locked nucleic acid (LNA) molecules, e.g., including [alpha]-L-LNAs. LNAs comprise ribonucleic acid analogues wherein the ribose ring is “locked” by a methylene bridge between the 2′-oxygen and the 4′-carbon—i.e., inhibitory nucleic acids containing at least one LNA monomer, that is, one 2′-O,4′-C-methylene-β-D-ribofuranosyl nucleotide. LNA bases form standard Watson-Crick base pairs but the locked configuration increases the rate and stability of the basepairing reaction (Jepsen et al., Oligonucleotides, 14, 130-146 (2004)). LNAs also have increased affinity to base pair with RNA as compared to DNA. These properties render LNAs especially useful as probes for fluorescence in situ hybridization (FISH) and comparative genomic hybridization, as knockdown tools for miRNAs, and as antisense oligonucleotides to target mRNAs or other RNAs, e.g., RNAs as described herein.

The LNA molecules can include molecules comprising 10-30, e.g., 12-24, e.g., 12, 13, 14, 15, 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the RNA. The LNA molecules can be chemically synthesized using methods known in the art.

The LNA molecules can be designed using any method known in the art; a number of algorithms are known, and are commercially available (e.g., on the internet, for example at exiqon.com). See, e.g., You et al., Nuc. Acids. Res. 34:e60 (2006); McTigue et al., Biochemistry 43:5388-405 (2004); and Levin et al., Nuc. Acids. Res. 34:e142 (2006). For example, “gene walk” methods, similar to those used to design antisense oligos, can be used to optimize the inhibitory activity of the LNA; for example, a series of inhibitory nucleic acids of 10-30 nucleotides spanning the length of a target RNA can be prepared, followed by testing for activity. Optionally, gaps, e.g., of 5-10 nucleotides or more, can be left between the LNAs to reduce the number of inhibitory nucleic acids synthesized and tested. GC content is preferably between about 30-60%. General guidelines for designing LNAs are known in the art; for example, LNA sequences will bind very tightly to other LNA sequences, so it is preferable to avoid significant complementarity within an LNA. Contiguous runs of more than four LNA residues, should be avoided where possible (for example, it may not be possible with very short (e.g., about 9-10 nt) inhibitory nucleic acids). In some embodiments, the LNAs are xylo-LNAs.

For additional information regarding LNAs see U.S. Pat. Nos. 6,268,490; 6,734,291; 6,770,748; 6,794,499; 7,034,133; 7,053,207; 7,060,809; 7,084,125; and 7,572,582; and U.S. Pre-Grant Pub. Nos. 20100267018; 20100261175; and 20100035968; Koshkin et al. Tetrahedron 54, 3607-3630 (1998); Obika et al. Tetrahedron Lett. 39, 5401-5404 (1998); Jepsen et al., Oligonucleotides 14:130-146 (2004); Kauppinen et al., Drug Disc. Today 2(3):287-290 (2005); and Ponting et al., Cell 136(4):629-641 (2009), and references cited therein.

### Making and Using Inhibitory Nucleic Acids

The nucleic acid sequences used to practice the methods described herein, whether RNA, cDNA, genomic DNA, vectors, viruses or hybrids thereof, can be isolated from a variety of sources, genetically engineered, amplified, and/or expressed/generated recombinantly. Recombinant nucleic acid sequences can be individually isolated or cloned and tested for a desired activity. Any recombinant expression system can be used, including e.g. in vitro, bacterial, fungal, mammalian, yeast, insect or plant cell expression systems.

Nucleic acid sequences of the invention can be inserted into delivery vectors and expressed from transcription units within the vectors. The recombinant vectors can be DNA plasmids or viral vectors. Generation of the vector construct can be accomplished using any suitable genetic engineering techniques well known in the art, including, without limitation, the standard techniques of PCR, oligonucleotide synthesis, restriction endonuclease digestion, ligation, transformation, plasmid purification, and DNA sequencing, for example as described in Sambrook et al. Molecular Cloning: A Laboratory Manual. (1989)), Coffin et al. (Retroviruses. (1997)) and “RNA Viruses: A Practical Approach” (Alan J. Cann, Ed., Oxford University Press, (2000)). As will be apparent to one of ordinary skill in the art, a variety of suitable vectors are available for transferring nucleic acids of the invention into cells. The selection of an appropriate vector to deliver nucleic acids and optimization of the conditions for insertion of the selected expression vector into the cell, are within the scope of one of ordinary skill in the art without the need for undue experimentation. Viral vectors comprise a nucleotide sequence having sequences for the production of recombinant virus in a packaging cell. Viral vectors expressing nucleic acids of the invention can be constructed based on viral backbones including, but not limited to, a retrovirus, lentivirus, adenovirus, adeno-associated virus, pox virus or alphavirus. The recombinant vectors capable of expressing the nucleic acids of the invention can be delivered as described herein, and persist in target cells (e.g., stable transformants).

Nucleic acid sequences used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Nucleic acid sequences of the invention can be stabilized against nucleolytic degradation such as by the incorporation of a modification, e.g., a nucleotide modification. For example, nucleic acid sequences of the invention includes a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” i.e., comprise nucleic acid analogues in which the ribose ring is “locked” by a methylene bridge connecting the 2′-O atom and the 4′-C atom (see, e.g., Kaupinnen et al., Drug Disc. Today 2(3):287-290 (2005); Koshkin et al., J. Am. Chem. Soc., 120(50):13252-13253 (1998)). For additional modifications see US 20100004320, US 20090298916, and US 20090143326.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook et al., Molecular Cloning; A Laboratory Manual 3d ed. (2001); Current Protocols in Molecular Biology, Ausubel et al., eds. (John Wiley & Sons, Inc., New York 2010); Kriegler, Gene Transfer and Expression: A Laboratory Manual (1990); Laboratory Techniques In Biochemistry And Molecular Biology: Hybridization With Nucleic Acid Probes, Part I Theory and Nucleic Acid Preparation, Tijssen, ed. Elsevier, N.Y. (1993).

### Pharmaceutical Compositions

The methods described herein can include the administration of pharmaceutical compositions and formulations comprising an inhibitor of XIST RNA and an inhibitor of an XIST-interacting protein, e.g., a chromatin-modifying protein, e.g., a small molecule inhibitor or an inhibitory nucleic acid such as a small inhibitory RNA (siRNA) or LNA that targets XIST RNA and/or a gene encoding XIST or an XIST-interacting protein, e.g., a chromatin-modifying protein, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342. The methods can include administration of a single composition comprising an inhibitor of XIST and an inhibitor of an XIST-interacting protein, e.g., a chromatin-modifying protein, or multiple compositions, e.g., each comprising one or both of an inhibitor of XIST and an inhibitor of an XIST-interacting protein, e.g., a chromatin-modifying protein.

In some embodiments, the compositions are formulated with a pharmaceutically acceptable carrier. The pharmaceutical compositions and formulations can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration of pharmaceuticals are well described in the scientific and patent literature, see, e.g., Remington: The Science and Practice of Pharmacy, 21st ed., 2005.

The inhibitory nucleic acids can be administered alone or as a component of a pharmaceutical formulation (composition). The compounds may be formulated for administration, in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the compositions of the invention include those suitable for intradermal, inhalation, oral/nasal, topical, parenteral, rectal, and/or intravaginal administration. The formulations may conveniently be presented in unit dosage form and may be prepared by any methods well known in the art of pharmacy. The amount of active ingredient (e.g., nucleic acid sequences of this invention) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g., an antigen specific T cell or humoral response.

Pharmaceutical formulations can be prepared according to any method known to the art for the manufacture of pharmaceuticals. Such drugs can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipient, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcellulose; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate. Push-fit capsules can contain active agents mixed with a filler or binders such as lactose or starches, lubricants such as talc or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., nucleic acid sequences of the invention) in admixture with excipients suitable for the manufacture of aqueous suspensions, e.g., for aqueous intradermal injections. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

In some embodiments, oil-based pharmaceuticals are used for administration of nucleic acid sequences of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102.

Pharmaceutical formulations can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. In alternative embodiments, these injectable oil-in-water emulsions of the invention comprise a paraffin oil, a sorbitan monooleate, an ethoxylated sorbitan monooleate and/or an ethoxylated sorbitan trioleate.

The pharmaceutical compounds can also be administered by in intranasal, intraocular and intravaginal routes including suppositories, insufflation, powders and aerosol formulations (for examples of steroid inhalants, see e.g., Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In some embodiments, the pharmaceutical compounds can be delivered transdermally, by a topical route, formulated as applicator sticks, solutions, suspensions, emulsions, gels, creams, ointments, pastes, jellies, paints, powders, and aerosols.

In some embodiments, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

In some embodiments, the pharmaceutical compounds can be parenterally administered, such as by intravenous (IV) administration or administration into a body cavity or lumen of an organ. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

In some embodiments, the pharmaceutical compounds and formulations can be lyophilized. Stable lyophilized formulations comprising an inhibitory nucleic acid can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. 20040028670.

The compositions and formulations can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587. As used in the present invention, the term “liposome” means a vesicle composed of amphiphilic lipids arranged in a bilayer or bilayers. Liposomes are unilamellar or multilamellar vesicles that have a membrane formed from a lipophilic material and an aqueous interior that contains the composition to be delivered. Cationic liposomes are positively charged liposomes that are believed to interact with negatively charged DNA molecules to form a stable complex. Liposomes that are pH-sensitive or negatively-charged are believed to entrap DNA rather than complex with it. Both cationic and noncationic liposomes have been used to deliver DNA to cells.

Liposomes can also include “sterically stabilized” liposomes, i.e., liposomes comprising one or more specialized lipids. When incorporated into liposomes, these specialized lipids result in liposomes with enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The formulations of the invention can be administered for prophylactic and/or therapeutic treatments. In some embodiments, for therapeutic applications, compositions are administered to a subject who is need of reduced triglyceride levels, or who is at risk of or has a disorder described herein, in an amount sufficient to cure, alleviate or partially arrest the clinical manifestations of the disorder or its complications; this can be called a therapeutically effective amount. For example, in some embodiments, pharmaceutical compositions of the invention are administered in an amount sufficient to decrease serum levels of triglycerides in the subject.

The amount of pharmaceutical composition adequate to accomplish this is a therapeutically effective dose. The dosage schedule and amounts effective for this use, i.e., the dosing regimen, will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; Remington: The Science and Practice of Pharmacy, 21st ed., 2005). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on for example: the dosage and frequency as required and tolerated by the patient, the degree and amount of therapeutic effect generated after each administration (e.g., effect on tumor size or growth), and the like. The formulations should provide a sufficient quantity of active agent to effectively treat, prevent or ameliorate conditions, diseases or symptoms.

In alternative embodiments, pharmaceutical formulations for oral administration are in a daily amount of between about 1 to 100 or more mg per kilogram of body weight per day. Lower dosages can be used, in contrast to administration orally, into the blood stream, into a body cavity or into a lumen of an organ. Substantially higher dosages can be used in topical or oral administration or administering by powders, spray or inhalation. Actual methods for preparing parenterally or non-parenterally administrable formulations will be known or apparent to those skilled in the art and are described in more detail in such publications as Remington: The Science and Practice of Pharmacy, 21st ed., 2005.

Various studies have reported successful mammalian dosing using complementary nucleic acid sequences. For example, Esau C., et al., (2006) Cell Metabolism, 3(2):87-98 reported dosing of normal mice with intraperitoneal doses of miR-122 antisense oligonucleotide ranging from 12.5 to 75 mg/kg twice weekly for 4 weeks. The mice appeared healthy and normal at the end of treatment, with no loss of body weight or reduced food intake. Plasma transaminase levels were in the normal range (AST ¾ 45, ALT ¾ 35) for all doses with the exception of the 75 mg/kg dose of miR-122 ASO, which showed a very mild increase in ALT and AST levels. They concluded that 50 mg/kg was an effective, non-toxic dose. Another study by Krützfeldt J., et al., (2005) Nature 438, 685-689, injected anatgomirs to silence miR-122 in mice using a total dose of 80, 160 or 240 mg per kg body weight. The highest dose resulted in a complete loss of miR-122 signal. In yet another study, locked nucleic acids (“LNAs”) were successfully applied in primates to silence miR-122. Elmen J., et al., (2008) Nature 452, 896-899, report that efficient silencing of miR-122 was achieved in primates by three doses of 10 mg kg-1 LNA-antimiR, leading to a long-lasting and reversible decrease in total plasma cholesterol without any evidence for LNA-associated toxicities or histopathological changes in the study animals.

In some embodiments, the methods described herein can include co-administration with other drugs or pharmaceuticals, e.g., compositions for providing cholesterol homeostasis. For example, the inhibitory nucleic acids can be co-administered with drugs for treating or reducing risk of a disorder described herein.

Disorders Associated with X-Inactivation

The present disclosure provides methods for treating X-linked diseases formulated by administering an inhibitor of an XIST RNA and an inhibitor of an XIST interacting protein, e.g., a small molecule inhibitor or an inhibitory nucleic acid such as a small inhibitory RNA (siRNA) or LNA that targets XIST or a gene encoding XIST or an XIST-interacting protein, e.g., a chromatin-modifying protein, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342, to disrupt silencing of genes controlled by the PRC2 sites (e.g., all of the genes within a cluster), or to disrupt silencing of one specific gene. This methodology is useful in X-linked disorders, e.g., in heterozygous women who retain a wild-type copy of a gene on the Xi (See, e.g., Lyon, Acta Paediatr Suppl. 2002; 91(439):107-12; Carrell and Willard, Nature. 434(7031):400-4 (2005); den Veyver, Semin Reprod Med. 19(2):183-91 (2001)). In females, reactivating a non-disease silent allele on the Xi would be therapeutic in many cases of X-linked disease, such as Rett Syndrome (caused by MECP2 mutations), Fabry's Disease (caused by GLA mutations), or X-linked hypophosphatemia (caused by mutation of PHEX). The methodology may also be utilized to treat male X-linked disease. In both females and males, upregulation of a hypomorphic or epigenetically silenced allele may alleviate disease phenotype, such as in Fragile X Syndrome, where the mechanism of epigenetic silencing of FMR1 may be similar to epigenetic silencing of a whole Xi in having many different types of heterochromatic marks.

As a result of X-inactivation, heterozygous females are mosaic for X-linked gene expression; some cells express genes from the maternal X and other cells express genes from the paternal X. The relative ratio of these two cell populations in a given female is frequently referred to as the “X-inactivation pattern.” One cell population may be at a selective growth disadvantage, resulting in clonal outgrowth of cells with one or the other parental X chromosome active; this can cause significant deviation or skewing from an expected mean X-inactivation pattern (i.e., 50:50). See, e.g., Plenge et al., Am. J. Hum. Genet. 71:168-173 (2002) and references cited therein.

The present methods can be used to treat disorders associated with X-inactivation, which includes those listed in Table 4. The methods include administering a an inhibitor of XIST RNA (e.g., an inhibitory nucleic acid such as a small inhibitory RNA (siRNA) or LNA that targets XIST) and an inhibitor of an XIST-interacting protein, e.g., a chromatin-modifying protein, e.g., a small molecule inhibitor, and optionally an inhibitory nucleic acid that specifically binds, or is complementary, to a strong or moderate binding site or a supRNA described in WO 2012/065143, WO 2012/087983, WO 2014/025887 and U.S. Ser. No. 62/010,342, i.e., a supRNA associated with the gene that causes the disorder, as shown in Table 4 and WO 2012/065143, WO 2012/087983, and WO 2014/025887.

TABLE 4
X Linked Disorders and Associated Genes

| Disorder | OMIM # | Locus | Gene |
|---|---|---|---|
| Dent's disease 1 | 300009 | Xp11.22 | CLCN5 |
| Testicular feminization syndrome | 300068 | Xq11-q12 | AR |
| Addison's disease with cerebral | 300100 | Xq28 | ABCD1 |
| sclerosis | | | |
| Adrenal hypoplasia | 300200 | XP21.3-p21.2 | DAX1 |
| siderius X-linked mental retardation | 300263 | Xp11.22 | PHF8 |
| syndrome | | | |
| Agammaglobulinaemia, Bruton type | 300300 | Xq21.3-q22 | BTK |
| Choroidoretinal degeneration | 300389 | Xp21.1 | RPGR |
| Choroidaemia | 300390 | Xq21.2 | CHM |
| Albinism, ocular | 300500 | Xp22.3 | OA1 |
| Dent's disease 2 | 300555 | Xq25-q26 | OCRL |
| fragile X syndrome | 300624 | Xq27.3 | FMR1 |
| Rett/Epileptic encephalopathy, early | 300672 | Xp22.13 | CDKL5 |
| infantile, 2 (CDKL5 deficiency | | | |
| disorder) | | | |
| Albinism-deafness syndrome | 300700 | Xq26.3-q27.1 | ADFN |
| paroxysmal nocturnal hemoglobinuria | 300818 | Xp22.2 | PIGA |
| Aldrich syndrome | 301000 | Xp11.23-p11.22 | WAS |
| Alport syndrome | 301050 | Xq22.3 | COL4A5 |
| Anaemia, hereditary hypochromic | 301300 | Xp11.21 | ALAS2 |
| Anemia, sideroblastic, with ataxia | 301310 | Xq13.3 | ABCB7 |
| Fabry disease | 301500 | Xq22 | GLA |
| Spinal muscular atrophy 2 | 301830 | Xp11.23 | UBA1 |
| Cataract, congenital | 302200 | Xp | CCT |
| Charcot-Marie-Tooth, peroneal | 302800 | Xq13.1 | GJB1 |
| Spastic paraplegia | 303350 | Xq28 | L1CAM |
| Colour blindness | 303800 | Xq28 | OPN1MW |
| Diabetes insipidus, nephrogenic | 304800 | Xq28 | AVPR2 |
| DDX3X syndrome | 300160 | Xp11.4 | DDX3X |
| Dyskeratosis congenita | 305000 | Xq28 | DKC1 |
| Ectodermal dysplasia, anhidrotic | 305100 | Xq12-q13.1 | ED1 |
| Faciogenital dysplasia (Aarskog | 305400 | Xp11.21 | FGD1 |
| syndrome) | | | |
| Glucose-6-phosphate dehydrogenase | 305900 | Xq28 | G6PD |
| deficiency | | | |
| Glycogen storage disease type VIII | 306000 | Xp22.2-p22.1 | PHKA2 |
| Gonadal dysgenesis (XY female type) | 306100 | Xp22.11-p21.2 | GDXY |
| Granulomatous disease (chronic) | 306400 | Xp21.1 | CYBB |
| Haemophilia A | 306700 | Xq28 | F8 |
| Haemophilia B | 306900 | Xq27.1-q27.2 | F9 |
| Hydrocephalus (aqueduct stenosis) | 307000 | Xq28 | L1CAM |
| Hypophosphataemic rickets | 307800 | Xp22.2-p22.1 | PHEX |
| Lesch-Nyhan syndrome (hypoxanthine- | 308000 | Xq26-q27.2 | HPRT1 |
| guanine-phosphoribosyl transferase | | | |
| deficiency) | | | |
| Incontinentia pigmenti | 308300 | Xq28 | IKBKG |
| Kallmann syndrome | 308700 | Xp22.3 | KAL1 |
| Keratosis follicularis spinulosa | 308800 | Xp22.1 | SAT |
| Lowe (oculocerebrorenal) syndrome | 309000 | Xq26.1 | OCRL |
| Menkes syndrome | 309400 | Xq12-q13 | ATP7A |
| Renpenning Syndrome | 309500 | Xp11.23 | PQBP1 |
| Mental retardation, with or without | 309530 | Xp11.3-q21.1 | MRX1 |
| fragile site (numerous specific types) | | | |
| Coffin-Lowry syndrome | 309580 | Xq13 | ATRX |
| Microphthalmia with multiple | 309800 | Xq27-q28 | MAA |
| anomalies (Lenz syndrome) | | | |
| Muscular dystrophy (Becker, Duchenne | 310300 | Xq28 | EMD |
| and Emery-Dreifuss types) | | | |
| Myotubular myopathy | 310400 | Xq28 | MTM1 |
| Night blindness, congenital stationary | 310500 | Xp11.4 | CSNB1 |
| Norrie's disease (pseudoglioma) | 310600 | Xp11.4 | NDP |
| Nystagmus, oculomotor or ‘jerky’ | 310700 | Xq26-q27 | NYS1 |
| Orofaciodigital syndrome (type I) | 311200 | Xp22.3-p22.2 | OFD1 |
| Ornithine transcarbamylase deficiency | 311250 | Xp21.1 | OTC |
| (type I hyperammonaemia) | | | |
| Phosphoglycerate kinase deficiency | 311800 | Xq13 | PGK1 |
| Phosphoribosylpyrophosphate | 311850 | Xq22-q24 | PRPS1 |
| synthetase deficiency | | | |
| Retinitis pigmentosa | 312610 | Xp21.1 | RPGR |
| Retinoschisis | 312700 | Xp22.2-p22.1 | RS1 |
| Rett syndrome | 312750 | Xq28, Xp22 | MECP2 |
| Muscular atrophy/Dihydrotestosterone | 313200 | Xq11-q12 | AR |
| receptor deficiency | | | |
| Spinal muscular atrophy | 313200 | Xq11-q12 | AR |
| Spondyloepiphyseal dysplasia tarda | 313400 | Xp22.2-p22.1 | SEDL |
| Thrombocytopenia, hereditary | 313900 | Xp11.23-p11.22 | WAS |
| Thyroxine-binding globulin, absence | 314200 | Xq22.2 | TBG |
| McLeod syndrome | 314850 | Xp21.1 | XK |

Portions of Table 4 were adapted in part from Germain, “Chapter 7: General aspects of X-linked diseases” in Fabry Disease: Perspectives from 5 Years of FOS. Mehta A, Beck M, Sunder-Plassmann G, editors. (Oxford: Oxford PharmaGenesis; 2006).

### EXAMPLES

The invention is further described in the following examples, which do not limit the scope of the invention described in the claims.

### Materials and Methods

The following materials and methods were used in the Examples, below.

### Design of Gapmer ASOs

Gapmers targeting XIST were designed following specific design algorithms (Exiqon), sequences in Table 1. 5-aza-2′-deoxycytidine (Aza) and other small molecules were purchased from Selleckchem or Tocris. I-BRD9 was obtained from SGC.

### Tissue Culture

Mecp2-Luc fibroblast cell lines were a generous gift from Dr. Bedalov. The clonal hybrid (cast/mus) cell line (EY. T4) was previously developed in the lab (Yildirim E et al. (2011) Nat Struct Mol Biol 19:56-61). Passage number was kept below 25, no further verification of cell line identity was performed. Human CDKL5 patient fibroblast cell lines were a generous gift from Dr. Sheridan. The clones (CDKL5wtXa, CDKL5mutXa) were previously developed by Dr. Roy Perlis. (Smita J et al. (2019) They were maintained in DMEM-glutamax (Gibco), supplemented with fetal bovine serum (FBS, 10%), non-essential amino acids (1X, Gibco), HEPES buffer (25 mM, Gibco), penicillin/streptomycin (1×, Gibco) and 2-Mercaptoethanol (Sigma). Transfection was performed with 20 nM ASO assisted by Lipofectamine LTX with Plus reagent (Thermo Fisher).

Imaging was done with a Nikon Eclipse TE2000-E equipped with a HamamatsuCCD camera. Image analysis was done with OpenLAB software (Agilent).

### Luciferase Assay

Immortalized clonal MEF cell line that carries an Mecp2: luciferase fusion gene on the Xi were used for conducting these experiments (Sripathy S, et al. (2017) Screen for reactivation of MeCP2 on the inactive X chromosome identifies the BMP/TGF-beta superfamily as a regulator of XIST expression. Proc Natl Acad Sci USA 114:1619-1624). Xa-Mecp2-Luc clone cell line was used in parallel for providing a scaling magnitude for normalizing Xi-driven luciferase signals, Cells were grown in a 12 well plate, trypsinized, counted, washed with PBS and dispensed in 20 ul of 1× cell culture lysis reagent (Promega). The mixture was vortexed and incubated for 5 min and then transferred to a zebra 96 well plate. The plate was read using a Perkin Elmer MicroBeta2 LumiJET that automatically adds 100 μl of Luciferase Assay Reagent (Promega) 2 sec before measuring the produced light for 10 sec. The corrected counts per second where divided by the number of cells for generating a luciferase-reactivation score per cell. For the initiating screen ASOs were used at 20 nM (transfected with lipofectamine) in combination with 0.5 uM Aza for 3 days. The reverse screen used 20 nM XIST ASO (transfected with lipofectamine) in combination with the small molecule inhibitors at different concentrations (see Table 5) for 3 days.

TABLE 5
concentrations and targets of the small molecules in the screen

| Inhibitor | Concentra- | |
|---|---|---|
| Name | tion (uM) | Target |
| etoposide | 0.25 | DNA topoisomerase 2 (TOP2) |
| EPZ6438 | 1 | Enhancer of zeste homolog 2 (EZH2) |
| PFI-3 | 5 | Transcription activator BRG1 (SMARCA4) |
| PRT4165 | 10 | Ring Finger Protein 1 (RING1) |
| I-BRD9 | 0.5 | Bromodomain Containing 9 (BRD9) |
| A366 | 0.05 | Euchromatic histone-lysine N- |
| | | methyltransferase 2 (G9A) |
| SGC-CBP30 | 0.5 | CREB Binding Protein (CREBBP) |
| olaparib | 0.05 | Poly ADP ribose polymerase (PARP) |
| JQ1 | 0.5 | Bromodomain and extraterminal domain |
| | | (BET) |
| barasertib | 0.01 | Aurora Kinase B (AURKB) |
| topotecan | 0.01 | DNA topoisomerase 1 (TOP1) |
| entinostat | 0.5 | Histone deacetylase 1&3 (HDAC1 & 3) |
| tubastatin a | 0.5 | Histone deacetylase 6 (HDAC6) |
| bestatin | 0.5 | Aminopeptidase |
| romidepsin | 0.5 | Histone deacetylase 1&2 (HDAC1 & 2) |
| rocilinostat | 0.05 | Histone deacetylase 6 (HDAC6) |
| vorinostat | 0.5 | Histone deacetylase (HDAC) |
| Azacitidine | 0.5 | DNA methyltransferase 1 (DNMT1) |
| decitabine | 0.5 | DNA methyltransferase 1 (DNMT1) |
| valproic acid | 0.5 | Histone deacetylase (HDAC) |
| VX680 | 1 | Aurora Kinase (AURK) |

qPCR

RNA was isolated by Trizol (Life Technologies) extraction (tissues were snap frozen in liquid nitrogen and ground with pestle and mortar or put in Trizol and homogenized using a Qiagen TissueLyser), treated with TurboDNAse for 30 min at 37° C. 2 μg RNA was used for each of the reverse transcriptase (and rt minus control) reactions (Superscript III, Invitrogen) followed by the SYBR green qPCR using the primers listed in table, with annealing temperature of 60° C. for 45 cycles. The relative efficiency of the reactivations was calculated by comparing to GAPDH or TBP RNA as the internal control in mouse experiment. The relative efficiency of the reactivations was calculated by comparing to RPL13a as the internal control in human cells.

### RNA-seq

Strand-specific RNA-seq was performed as previously described (Kung J T et al. (2015) Mol Cell 57:361-375; Minajigi A et al. (2015) Science, aab2276-12). All libraries were sequenced with Illumina HiSeq, generating 28-54 millions paired-end 50 nucleotide reads per sample. RNA-seq reads were aligned allele-specifically to 129S1/SvJm (mus) and CAST/Eih (cas) genome using TopHat2 (Kim D, et al. (2013) Genome Biol 14:R36). After removal of PCR duplicates, all unique reads mapped to the exons of each gene were quantified by Homer (Heinz S, et al. (2010) Mol Cell 38:576-589). For non-allelic analysis, we used all reads (comp reads, which contain both allele-specific reads and reads that do not overlap with SNPs) to perform normalized differential expression analyses by DESeq (Anders and Huber (2010) Genome Biol 11:R106). To compare the fold change of autosomal and X-linked genes, only genes with FPKM≥1 were considered (Yang et al. (2016) Genes Dev 30:1747-1760). Upregulated genes were defined as genes with fold change>1.2. For allele-specific analysis, we defined % mus as the percentage of mus-specific exonic reads in all allele-specific (mus-specific+cas-specific) exonic reads of each transcript. For classification of X-linked genes, we defined expressed genes as genes having non-zero FPKM in all samples. Allele-assessable genes were defined as active genes that have more than 12 allele-specific reads in all samples (Pinter S F and Colognori D (2015) Genetics 200:537-549). It has been described that a small fraction of genes overlap with incorrectly annotated SNPs and produce unexpected allelic skewing (Pinter S F and Colognori D (2015) Genetics 200:537-549; Calabrese J M et al. (2012) Cell 151:951-963). These genes were identified by analyzing a published RNA-seq dataset of tail-tip fibroblasts (TTF) from pure Mus castaneous background. Allele-assessable genes having % mus greater than 9.09% in the pure cas TTF were considered as genes with miscalled SNPs. Genes that are qualified for allele-specific analysis (qualified genes) were defined as genes that were allele-assessable and were not genes with miscalled SNPs. Among qualified genes, we defined escapees as genes whose expression from the Xi is greater than 10% of the expression from the Xa (Yang F et al. (2010) Genome Res 20:614-622) in wild-type hybrid MEF treated with control ASO. Genes subjected to X-inactivation (X-inactivated genes) were defined as expressed and allele-assessable genes that were not genes with miscalled SNPs and escapees. The cumulative distribution plots, histograms, heat maps, and scatter plots were constructed with R, ggplot2, and Gviz package (www.R-project.org). To visualize RNA-seq coverage, we generated strand-resolved fpm-normalized bigWig files from the raw RNA-seq reads for all reads (comp), mus-specific (mus) reads, and cas-specific (cas) reads separately, which were displayed using IGV with scales indicated in each tract.

### Mouse Husbandry

Mouse husbandry was carried out as stipulated by the Massachusetts Hospital

Institutional Animal Care and Use Committee (IACUC) and all animal experiments were approved by them. Moribund animals (sacrificed per IACUC) were included in the data as deceased animals.

XIST2^{lox}/XIST2^{lox} mice (129Sv/Jae strain) were a gift of R. Jaenisch. Nestin-Cre mice (B6.Cg-Tg(Nes-cre)1Kln/J) were a gift from R. Kelleher. To generate XISTΔ/+ mice, we crossed XIST2^{lox}/XIST2^{lox} females to Nest-Cre males. To generate homozygous mutants, we crossed XIST2^{lox}/XIST2^{lox} females to XIST2^{lox}/Y;Nest-Cre males. Mice were screened by PCR for Nest-Cre and XIST2^{lox} alleles using the primers in Tables 6 and 7.

TABLE 6
primers for qPCR

| Name | Sequence (5′ to 3′) | SEQ ID NO: |
|---|---|---|
| Tbp F | ACGGACAACTGCGTTGATTTT | 46. |
| Tbp R | ACTTAGCTGGGAAGCCCAAC | 47. |
| GapdH F | ATGAATACGGCTACAGCAACAGG | 48. |
| GapdH R | CTCTTGCTCAGTGTCCTTGCTG | 49. |
| XIST F | CAGAGTAGCGAGGACTTGAAGAG | 50. |
| XIST R | GCTGGTTCGTCTATCTTGTGGG | 51. |
| Luc F | TCTAAGGAAGTCGGGGAAGC | 52. |
| Luc R | CCCTCGGGTGTAATCAGAAT | 53. |

TABLE 7
primers for mouse genotyping

| | | SEQ |
|---|---|---|
| | | ID |
| Name | Sequence (5′ to 3′) | NO: |
| XIST F | GTGCCATATCAGTGAGCTCTCG | 54. |
| XIST2lox R | AACCAAGGTTGAGAGAGCAAA | 55. |
| XIST1lox R | TGTCACCTACCAATGAGAGATCC | 56. |
| Cre F | GCGGTCTGGCAGTAAAAACTATC | 57. |
| Cre R | GTGAAACAGCATTGCTGTCACTT | 58. |
| Cre ICF | CTAGGCCACAGAATTGAAAGATCT | 59. |
| Cre ICR | GTAGGTGGAAATTCTAGCATCATCC | 60. |
| Mecp2 F | AAATTGGGTTACACCGCTGA | 61. |
| Mecp2 R mut | CCACCTAGCCTGCCTGTACT | 62. |
| Mecp2 R WT | CTGTATCCTTGGGTCAAGCTG | 63. |
| Tsix F | GGAGAAGCCATTTTCCATCA | 64. |
| Tsix mut R | ACGGAACGCAGTACCAAAAT | 65. |
| Tsix WT R | CAAAAATCCCCAAGAATGTGA | 66. |
| Neo F | CGTTGGCTACCCGTGATATT | 67. |
| Neo R | TCAGAAGAACTCGTCAAGAAGG | 68. |
| Sex X F | GGTAACAATTTTCCCGCCATGTG | 69. |
| Sex X R | GGAAATAAACGGAACGCAGTACC | 70. |
| Sex Y F | GACTAGACATGTCTTAACATCTGTCC | 71. |
| Sex Y R | CCTATTGCATGGACAGCAGCTTATG | 72. |

TABLE 8
primers for qPCR for human CDKL5 experiment

| | | SEQ |
|---|---|---|
| | | ID |
| Name | Sequence (5′ to 3′) | NO: |
| RPL13a_F | ACCCTGGAGGAGAAGAGGA | 81. |
| RPL13a_R | AGGCAACGCATGAGGAATTA | 82. |
| hXIST_F | TTGCCCTACTAGCTCCTCGGAC | 83. |
| hXIST_R | TTCTCCAGATAGCTGGCAACC | 84. |
| CDKL5_F | CCACACCTTCTTAGCCCAAA | 85. |
| CDKL5_RwA | CTAGAGGACTGGGGAATTGTATC | 86. |
| CDKL5_RmutA | CCTAGAGGACTGGGGAATTGTAC | 87. |

### Behavioral Testing

Blinding in these experiments at this stage was not possible, so randomization was not performed. Littermates were used as control. The mice were kept in strict 12h light/dark cycles. All behavior analysis was performed during the light cycle in a dedicated behavior room, where mice where acclimatized for at least 20 min before the experiment. All mice were naïve to the test. Behavior tests were performed with the Mecp2 deletion mice at 7 weeks of age, with the XIST deletion mice at 1 year of age.

### Open Field Test

The behavior of a mouse placed in a box with transparent walls was observed, which allows to assay general locomotor activity and anxiety. Individual mice were placed in the corner of a commercial open field activity arena (27×27 cm, Med Associates Inc.) which consists of a lit open area equipped with infrared beams on the side to track movements in x-y and z and allowed to move freely for 1 h, divided in blocks of 15 min. Total distance traveled, ambulatory time, ambulatory counts, stereotypy time, stereotypy counts, resting time, vertical counts, vertical time, zone entries, zone time, jump counts, jump time, average velocity, and ambulatory episodes were recorded and analyzed with automated software for each test mouse throughout the 60 min. test session. The distance traveled provides a measure of general activity and amount of time spent in the center (middle 20×20 cm) versus the edges of the arena, where the mouse feels more comfortable shielded by the walls measures anxiety.

### Rotarod Test

The mice were placed on top of a beam in a commercial rotorod apparatus (Ugo Basile) facing away from the experimenter's view. The beam was rotated such that forward locomotion is necessary to avoid falling off the beam. The rotorod is accelerated gradually from 4 to 40 rpm over a 5 min trial. One daily session of 3 trials with a minimal 15 min interval were conducted. Sessions were repeated for 3 days (total of 9 trials). If the mouse clings to the rod without moving (passive rotation) for two complete revolutions, it is considered to have fallen.

### Elevated Plus Maze Test

In this assay, mice are put in a plus-shaped maze (Med Associates) that has 4 alternating open and closed (walled) arms arranged perpendicularly and is elevated approximately 50 cm above the floor. The test is based on the innate drive of mice to explore novel environments while avoiding exposed, bright and unprotected environments. Each mouse was placed in the center hub of the maze (where the 4 arms meet) with its nose pointing inside a closed arm. Movement was recorded using a video tracking system for 10 minutes. The latency to first entry into an open arm and the time spent in the closed arms (measures of anxiety-like behavior), as well as total number of arm entries (open and closed, an indicator of hyperactivity), is recorded. Increased latency to enter the open arms, or increased time spent in the closed arms, indicates increased anxiety-like behavior.

### Aza/Decitabine Three Pulse Treatment

Decitabine was administered to the XIST2lox, Nestin-Cre F2 generation, by IP injection at 5 weeks old. Three injections 100 ul per 10 g of 0.033 mg/ml in sterile saline (or just sterile saline as control) were given over the course of a week (each injection separated by 2 days). Both XIST2lox/2lox and XIST Δ/Δ were injected and were randomly assigned to the treatment group. No specific randomization protocol was followed. RNA from the brain and liver were harvested (as described before) at 7 weeks of age (2 weeks after the first injection).

### Tissue Sectioning

The tissue was imbedded in TOC and frozen in a slurry of dry ice with isopentane. The obtained blocks were sliced at 8 micron with a cryostat.

### Fluorescence In Situ Hybridization (FISH)

A tissue section was immobilized on a glass slide, rinsed in cold PBS (5 min), pre-extracted in 0.5% CSKT on ice (6 min), fixed with 4% paraformaldehyde in PBS at room temperature (10 min) and then stored or washed in 70% EtOH. For hybridization the slide was dehydrated through sequential washing in 80%, 90% and 100% EtOH (2 min) and air-drying. DNA probe (Alexa 647-labeled oligonucleotide probes as described before) (Sunwoo H, Wu J Y, Lee J T (2015) Proc Natl Acad Sci USA 112:E4216—E4225) was then added to the slide, which was covered and incubated for 5h at 37° C. After incubation the slide was washed 3 times with 50% formamide/2×SSC pH7.4 at 45° C. (5 min), 3 times with 0.5×SSC at 45° C. (5 min) and air-dried. The slide was then mounted with dapi containing antifade Vectashield (Vector Laboratories) and viewed under a Nikon Eclipse 90i microscope and Hamamatsu CCD camera. Image analysis (automated contrast enhancement for each channel in the whole image) was performed using Velocity (Perkin-Elmer).

### Statistics

Unless stated otherwise, error bars represent the standard error on the mean. For comparing 2 groups, p-values were calculated with the two sided T-test with equal variance. Variance was checked to be indeed equal with Levine's test and normality of the data was checked by looking at its representation in a histogram, its Q-Q plot and performing 2 tests of normality: Kolmogorov-Smirnov and Shapiro-Wilk. When this pointed out a possibility of non-normal distribution, the Mann-Whitney U test was performed. For comparing more than 2 groups one-way ANOVA test was performed. In case of unequal sample size, variance or a non-normal distribution, the Brown Forsythe test was performed. In the cumulative density plots p values were calculated using the Wilcoxon rank sum test.

### Example 1. Pharmacological Synergy Through a Mixed Modality Approach

While the pharmaceutical industry has focused almost exclusively on targeting proteins, long noncoding RNAs (lncRNA) have become increasingly attractive as pharmacological targets (Matsui M and Corey D R. Nat Rev Drug Discov. 2017; 16:167-179). With improving ASO technology, lncRNAs are now also pharmacologically accessible. ASOs are high molecular weight compounds that have been optimized over the past 50 years through chemical modifications to acquire greater stability, selectivity, and bioavailability (Bennett C F and Swayze E E. Annu Rev Pharmacol Toxicol. 2010; 50:259-293; Southwell A L et al. Trends Mol Med. 2012; 18:634-643). Since ASOs bind their target through Watson-Crick basepairing interactions, they can be rationally designed and hit previously “undruggable” targets. Notably, ASO technology has achieved success in treating hypercholesterolemia (Kynamro™) and spinal muscular atrophy (Spinraza™).

No candidate drug so far has achieved anything close to 1% MECP2 protein restoration. We asked whether an ASO, alone or in combination with an inhibitor of an XIST-interacting protein, could also be developed for Xi-reactivation of MECP2. We screened a small ASO library against various targets of potential interest, including XIST RNA and an antisense transcript to Mecp2 (Mecp2-as) (ASOs listed in Table 1).

In designing the ASOs, we chose phosporothioate backbone and locked nucleic acid (LNA™) chemistry (Wahlestedt C et al. Potent and nontoxic antisense oligonucleotides containing locked nucleic acids. Proc Natl Acad Sci USA. 2000; 97:5633-5638) for its in vivo and in vitro stability, and increased affinity and selectivity for RNA targets. All were designed as gapmers, with unmodified deoxyribonucleosides in the center flanked by 5′ and 3′ terminal locked nucleosides, to direct RNAse-H-mediated cleavage of the target transcript. We tested each ASO on an immortalized clonal mouse fibroblast cell line carrying an Mecp2:Luciferase knock-in reporter on the Xi (Sripathy S et al. Proc Natl Acad Sci USA. 2017; 114:1619-1624; Lessing D et al. Proc Natl Acad Sci USA. 2016; 113:14366-14371). The luciferase reporter provides a highly sensitive enzymatic detection method with a large dynamic range. Because previous studies provide strong support for synergistic Xi-reactivation (Csankovszki G et al. J Cell Biol. 2001; 153:773-784; Lessing D et al. Proc Natl Acad Sci USA. 2016; 113: 14366-14371; Minkovsky A et al. Epigenetics Chromatin. 2015; 8:42; Minajigi A, et al. Science. 2015; 349: aab2276-12), we examined the efficacy of each ASO in the presence of 0.5 uM decitabine (“Aza”; 5-aza-2′-deoxycytidine) for three days.

The XIST gapmer ASOs were able to reduce Aza combinations with ASOs against Mecp2-as or various nearby ASOs yielded inconsistent, low, or no Mecp2:Luciferase reactivation relative to untreated samples or Aza-only samples. XIST was depleted by >95% after 3-5 days (FIG. 2A). By itself, the ASO did not upregulate Mecp2, but when combined with Aza, we observed up to 30,000-fold upregulation in fibroblasts—equivalent to 3-5% of the active X (Xa) (FIG. 2B).

### Example 2. Female RTT Mouse Model

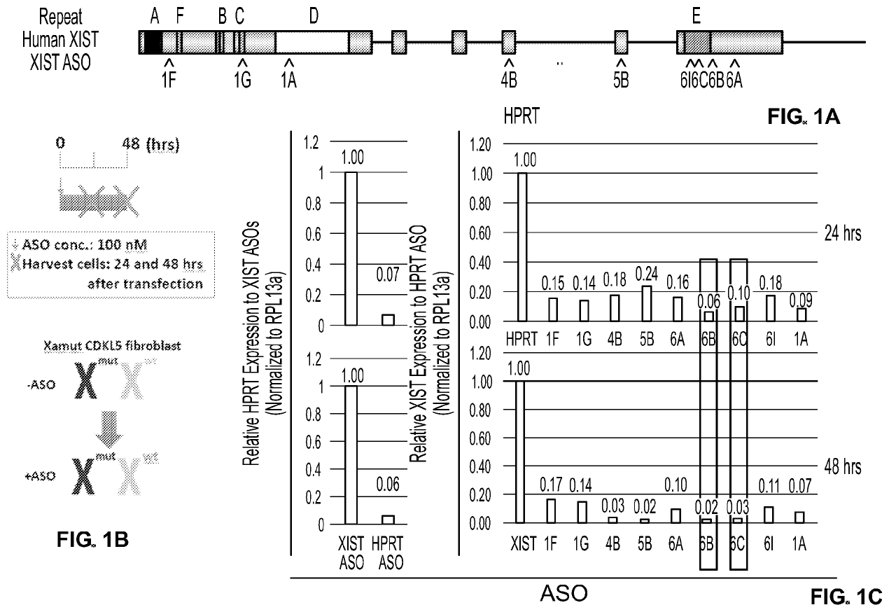

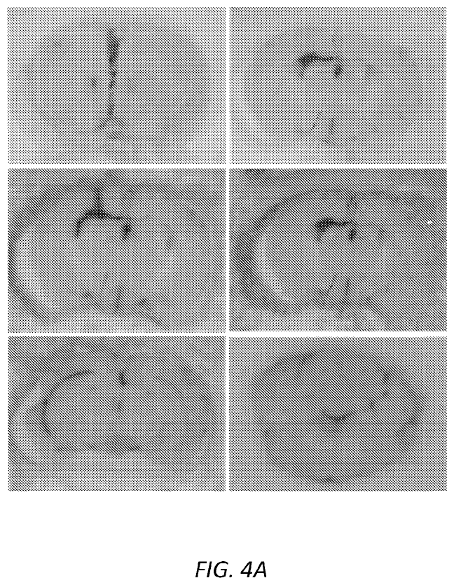

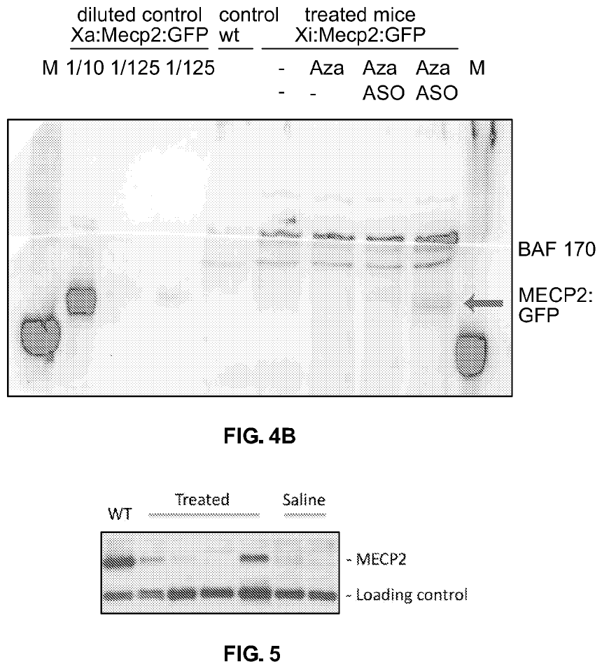

To study the X-reactivation platform and test candidate drugs, a female RTT model is needed. RTT research has relied mostly on male animals, as female Mecp2−/+ animals have mild and variable disease symptoms later in life (Guy et al., Nature genetics 27, 322-326, (2001); Chen et al., Nature genetics 27, 327-331, (2001); Shahbazian et al. Neuron 35, 243-254, (2002); Katz et al., Disease models & mechanisms 5, 733-745, (2012); Samaco et al., Human Molecular Genetics 22, 96-109, (2013); Wang et al., Journal of Neurology & Neurophysiology 03, 2-5, (2012)), but without an Xi, they are not appropriate subjects for testing Xi-reactivation. By crossing in the Tsix mutation, we skewed the pattern of XCI, so expression is favored from the X-chromosome with the mutation and nearly all cells are deficient for MECP2 protein. Indeed, these females phenocopied the severe disease of male RTT mice, both in neuromuscular function (gait & rotarod analyses) and lifespan (Carrette et al., Proc Natl Acad Sci USA 115: 8185-8190, 2018; FIGS. 1, 2, 4, and Movies S1-S9). Moreover, they engaged in obsessive grooming (OCB, obsessive compulsive behavior) resulting in severe injuries (SIB, self-injurious behavior) (Carrette et al., Proc Natl Acad Sci USA 115: 8185-8190, 2018; FIGS. 5E-F, and Movies S10-S11)—behaviors frequently observed in RTT and other autistic children (Minshawi et al., Psychol Res Behav Manag 7, 125-136, (2014)). These mice can be used, e.g., for pre-clinical testing of drug candidate(s).

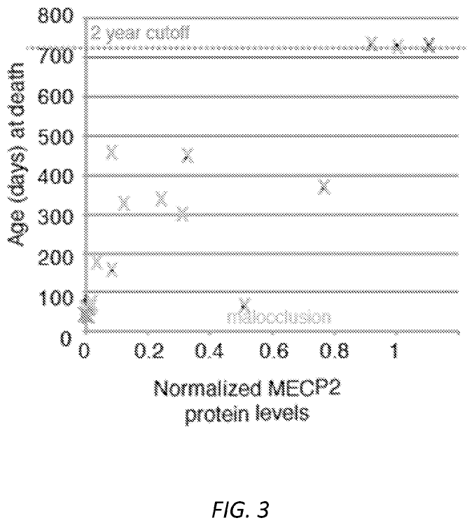

In the RTT female mouse model, we observed some variability in the achieved degree of skewing, causing variability in MECP2 expression in the brain. This finding presented an unprecedented opportunity to correlate MECP2 levels with severity of RTT. Intriguingly, there was a correlation between the MECP2 level and lifespan (FIGS. 2A-2B). MECP2 restoration to only 5-10% of the brain extended life 3- to 10-fold, with accompanying neuromotor improvement (Carrette et al., Proc Natl Acad Sci USA 115: 8185-8190, 2018; FIG. 3).

This level approached the amount of increased expression that we achieved with administration of an ASO plus a DNMT1 inhibitor in mice (FIGS. 2A-2B). These data suggest that targeting XIST RNA together with inhibition of DNA methylation is an effective method of achieving partial Xi-reactivation. Thus, even a modest restoration of MECP2 protein has therapeutic benefit.

### Example 3. CDKL5 Upregulation in Human CDKL5 Patient Cells

Without being bound by theory, MECP2 can be reactivated in mouse cells and RTT mouse model, which may suggest similar reactivation on human patient cells. To study the X-reactivation in human platform and test candidate drugs, experiments used a human CDKL5 patient fibroblast line. In human cell reactivation experiments, CDKL5 gene was targeted.

Mutations in CDKL5 are associated with an X-linked disorder, CDKL5 deficiency disorder, which is a variant of Rett syndrome, also known as early infantile Epileptic encephalopathy. Previous study shows that CDKL5 protein replacement in CDKL5-null mouse models rescued some behavioral symptoms and neuroanatomical abnormalities (Trazzi et al 2018). This is critical because it indicates that damage caused by loss of CDKL5 during neuronal development is largely reversible and opens up multiple avenues for therapeutics.

To test the XIST ASO plus decitabine combinations and look for potential Xi reactivation of CDKL5, experiments were conducted in CDKL5 clonal patient fibroblasts carrying mutation on the Xa. Experiments examined the efficacy of ASO 6A and 6C in the presence of 0.5 uM decitabine for three days. The human XIST ASO 6A and 6C target EXON6 at rep E, as shown in FIG. 1A. The human XIST ASOs target the following sequences: 6A:TATGGCCCACAGTCTAAAGT (SEQ ID NO: 1) and 6C: TTGGCCTTGTGTCACAAGTC (SEQ ID NO:12).

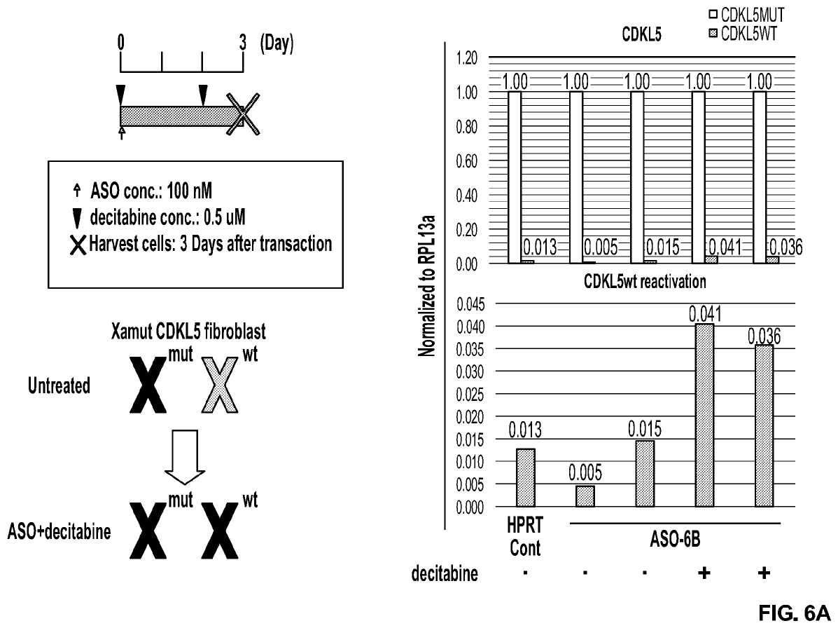

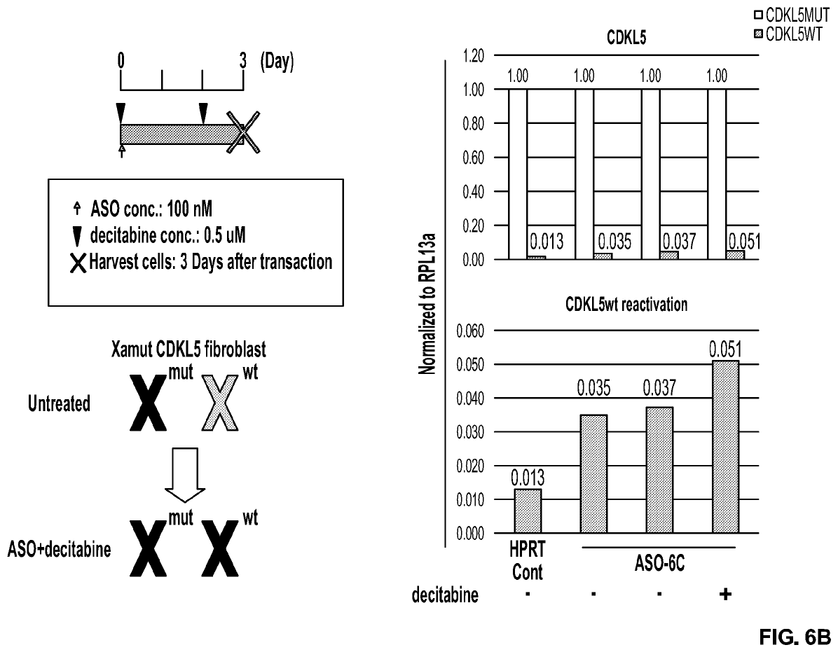

Allele-specific primer sets used to measure CDKL5 expression from the active allele (CDKL5mut) and inactive allele (CDKL5 wt). CDKL5 expression was normalized to RPL13a, relative to HPRT treated condition after 3 days of treatments. CDKL5 wt reactivation was normalized to CDKL5 expression from the Xa chromosome. By itself, the ASOs did not significantly upregulate CDKL5, but when combined with decitabine, there was an observed 2-5% upregulation of the CDKL5 Xi in fibroblasts (FIGS. 6A-6B). Thus, targeting XIST RNA together with inhibition of DNA methylation is an effective method of achieving partial Xi-reactivation in human fibroblasts.

Furthermore, to see better CDKL5 Xi reactivation, we examined the efficacy of XIST ASO 1U and DNMT1 ASO in the presence of 1 uM RG-108 for 5 days. The human XIST ASO 1U targets EXON1 at rep D. The human XIST ASOs target the following sequences: 1U:CTTACAACTGTGCACCTTGA (SEQ ID NO: 15) and human DNMT1 ASO targets the following sequences: DNMT: TCAAGTTGAGGCCAGAAGGA (SEQ ID NO: 80). There was an observed 2-2.5% reactivation with a combination of XIST ASO 1U and DNMT1 ASO without 1 uM RG-108.

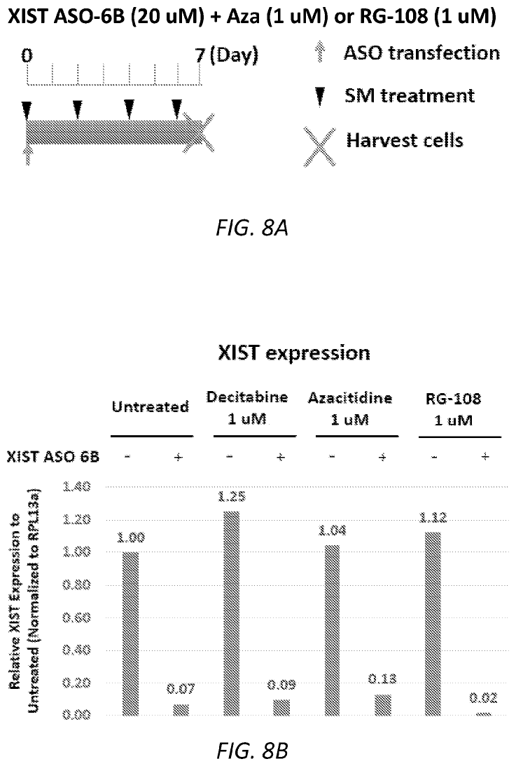

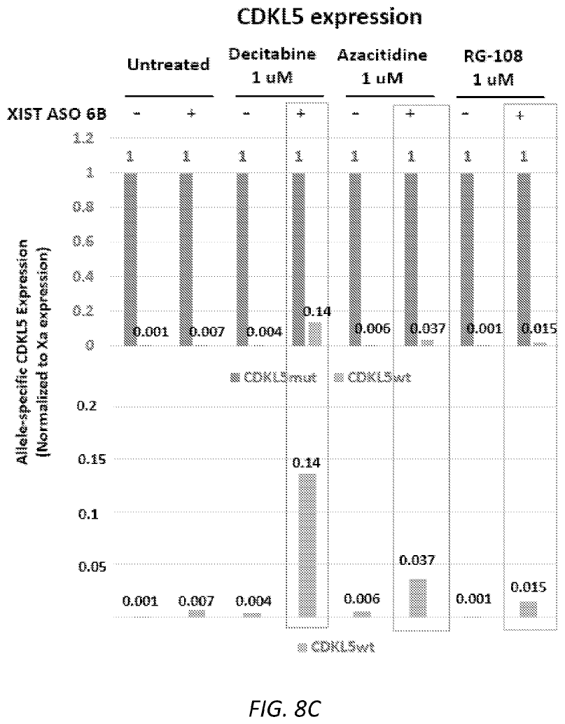

Additional experiments were performed to demonstrate allele-specific Xi reactivation. The human CDKL5 patient fibroblast line carrying a mutation on the Xa was treated with ASOs to XIST and three DNMTi small molecules (Decitabine, Azacitidine, or RG-108). On day 0, cells were treated with 20 nM of XIST ASO-6B (lipofectamine transfection), and 1 uM of a DNMTi every 2 days (days 0, 2, 4, and 6). On day 7, cells were harvested for qPCR analysis (FIG. 8A). qPCR results show successful decrease in XIST expression after 7 days treatment with 20 nM ASO-6B with and without 1 uM of each of DNMTi (Decitabine, Azacitidine, RG-108). As expected, XIST expression was decreased in all experiments where cells were treated with 20 nM of XIST ASO-6B (FIG. 8B). qPCR results showed allele-specific reactivation of CDKL5 after 7 days treatment with 20 nM ASO-6B and 1 uM of each of DNMTi (Decitabine, Azacitidine, or RG-108). CDKL5 wt reactivation was normalized to CDKL5mut expression from the Xa chromosome, and the results showed 14% CDKL5 reactivation when XIST ASO 6B was combined with decitabine, 3.7% CDKL5 reactivation when XIST ASO 6B was combined with Azacitidine, and 1.5% CDKL5 reactivation when XIST ASO 6B was combined with RG-108. Results showed 0.7% reactivation when cells were treated with 20 nM ASO-6B (FIG. 8C).

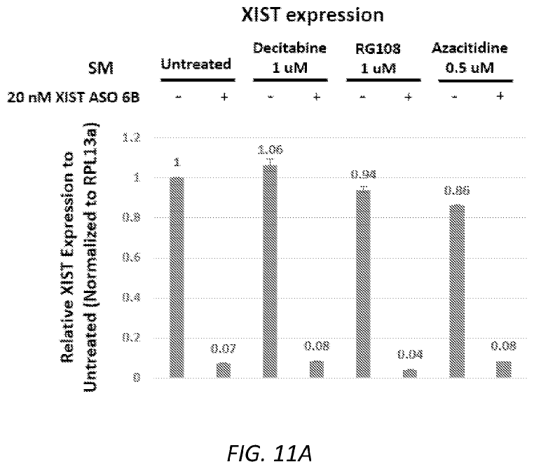

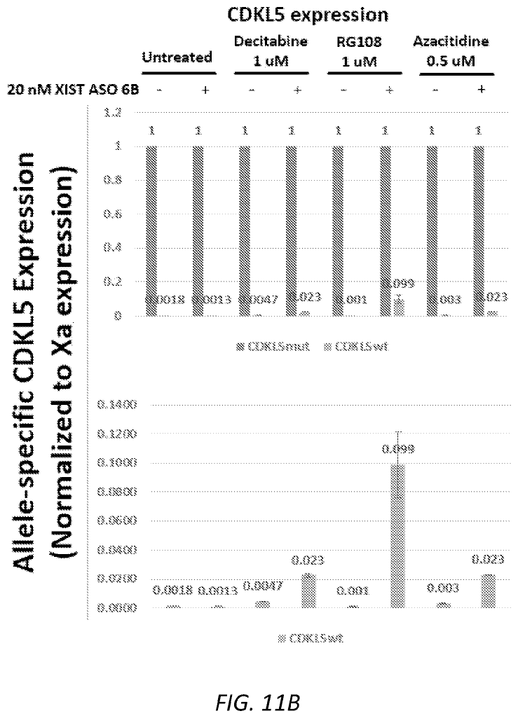

A similar experiment was done in triplicate, where a human CDKL5 patient fibroblast line carrying mutation on the Xa was treated with 20 nM XIST ASO 6B (added at day 0) with or without one of three DNMTi (1 uM Decitabine, 0.5 uM Azacitidine, 1 uM RG-108; added at days 0 and 2). Cells were harvested at Day 4. Again, qPCR results showed decreased XIST expression after 4 days treatment with 20 nM ASO-6B with or without each of DNMTi molecules (1 uM Decitabine, 0.5 uM Azacitidine, 1 uM RG-108; FIG. 11A). The qPCR results showed percentage of allele-specific CDKL5 wt reactivation after 4 days. The results showed showed 9.9% CDKL5 reactivation when 20 nM XIST ASO 6B was combined with 1 uM RG-108, 2.3% CDKL5 reactivation when 20 nM XIST ASO 6B was combined with 0.5 uM Azacitidine, and 2.3% CDKL5 reactivation when 20 nM XIST ASO 6B was combined with 1 uM Decitabine (FIG. 11B). Results showed 0.13% reactivation when cells were treated with 20 nM ASO-6B (FIG. 11B). Data shown are the average of three experiments.

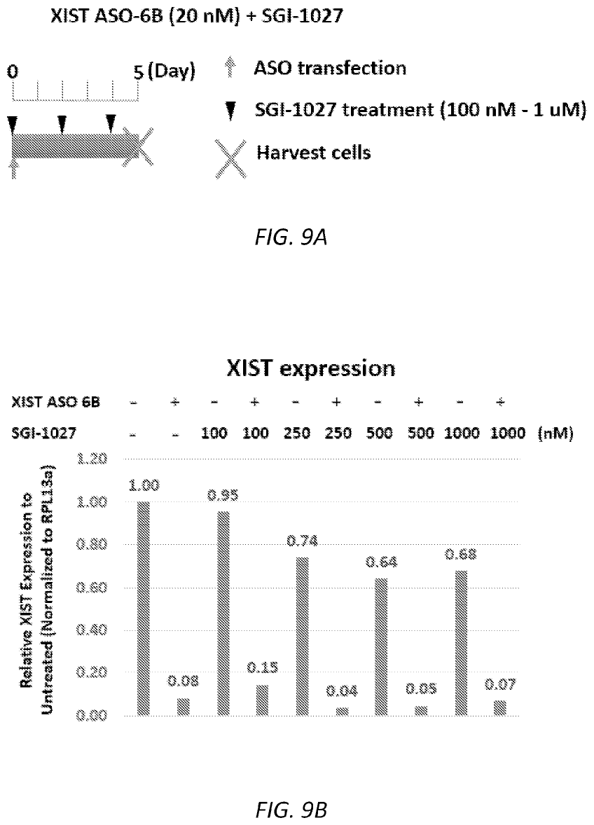

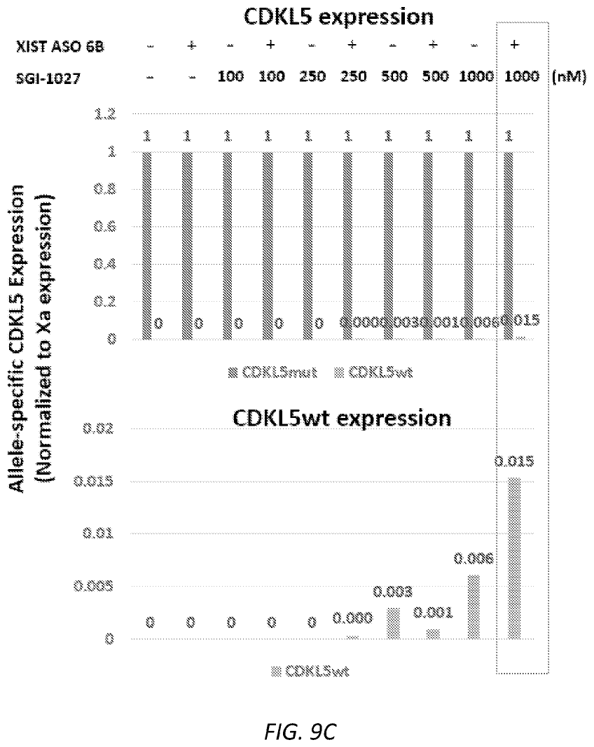

Experiments were also conducted to test Xi reactivation in human CDKL5 patient's fibroblast with XIST ASO-6B and DNMT inhibitor SGI-1027 co-treatment. A human CDKL5 patient fibroblast line carrying mutation on the Xa was treated with an ASO to XIST and a DNMTi. After 5 days of treatment with 20 nM XIST ASO-6B (added once at day 0; lipofectamine transfection) and 100 nM, 250 nM, 500 nM, or 1.0 uM SGI-1027 (added every 2 days; days 0, 2, and 4), cells were harvested for qPCR analysis (FIG. 9A). The qPCR results showed 1.5% CDKL5 reactivation when 20 nM XIST ASO 6B was combined with 1 uM SGI-1027 (FIG. 9C, box).

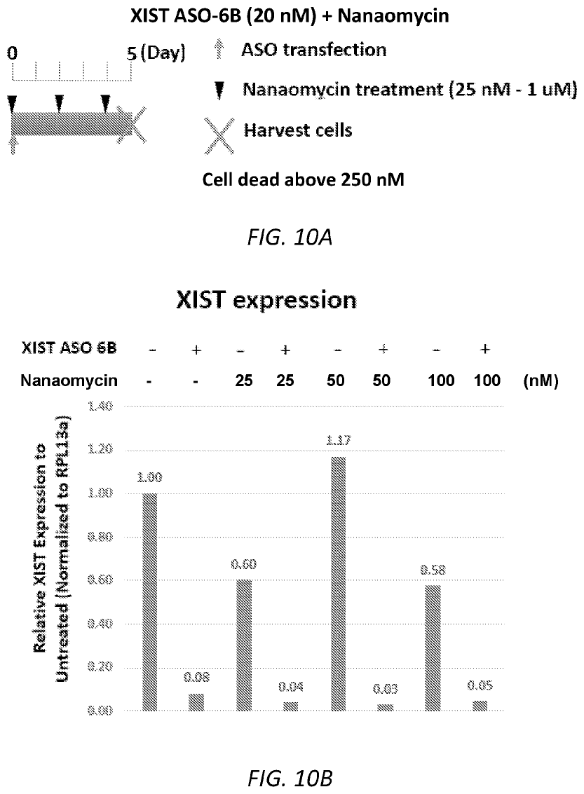

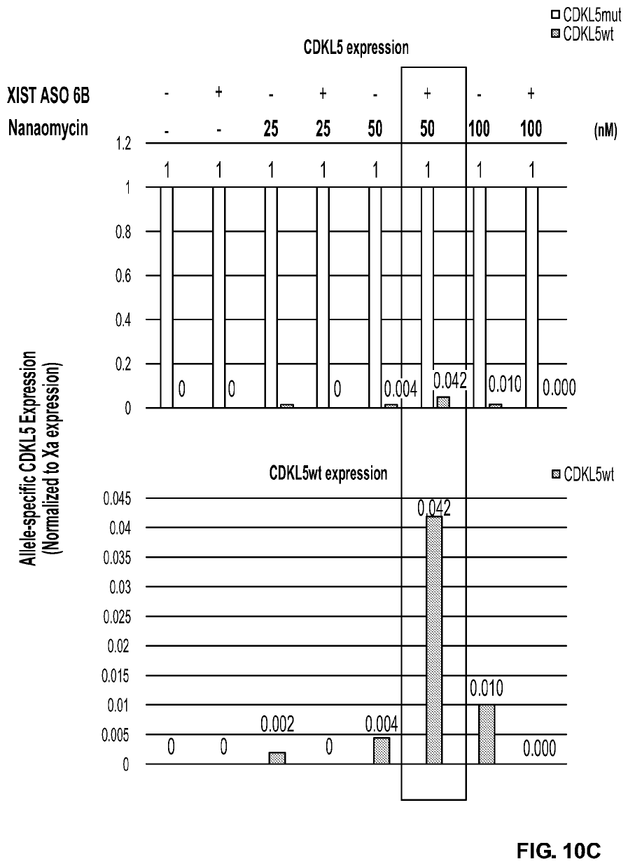

Experiments were also conducted to test Xi reactivation in human CDKL5 patient's fibroblast with XIST ASO-6B and DNMT3 inhibitor, Nanaomycin, co-treatment. A human CDKL5 patient fibroblast line carrying mutation on the Xa was treated with an ASO to XIST and a DNMT3i, nanaomycin A. After 5 days of treatment with 20 nM XIST ASO-6B (added once at day 0; lipofectamine transfection) and 25 nM-1 uM nanaomycin A (added every 2 days; days 0, 2, and 4), cells were harvested for qPCR analysis (FIG. 10A). Nanaomycin is very toxic, and the cells were dead in concentration above 250 nM (data not shown). The qPCR results showed 4.2% CDKL5 reactivation when 20 nM XIST ASO 6B was combined with 50 nM nanaomycin A (FIG. 10C, box).

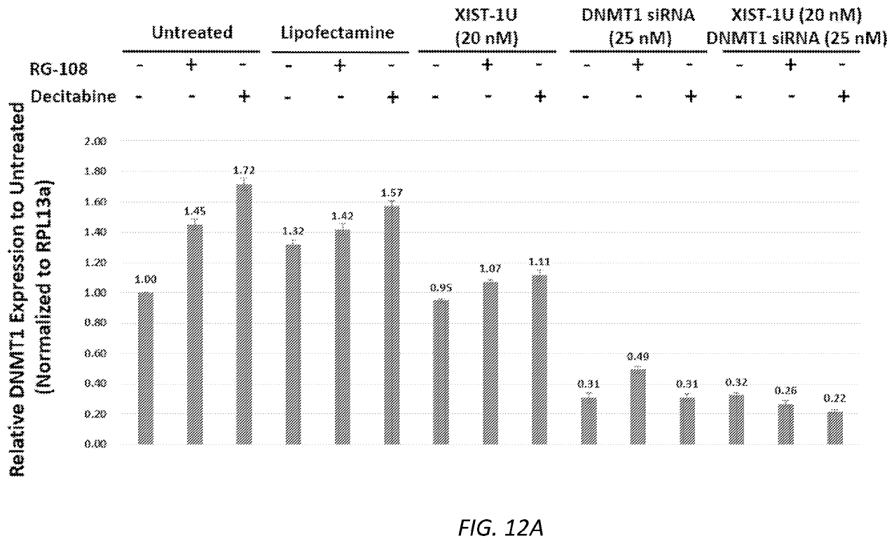

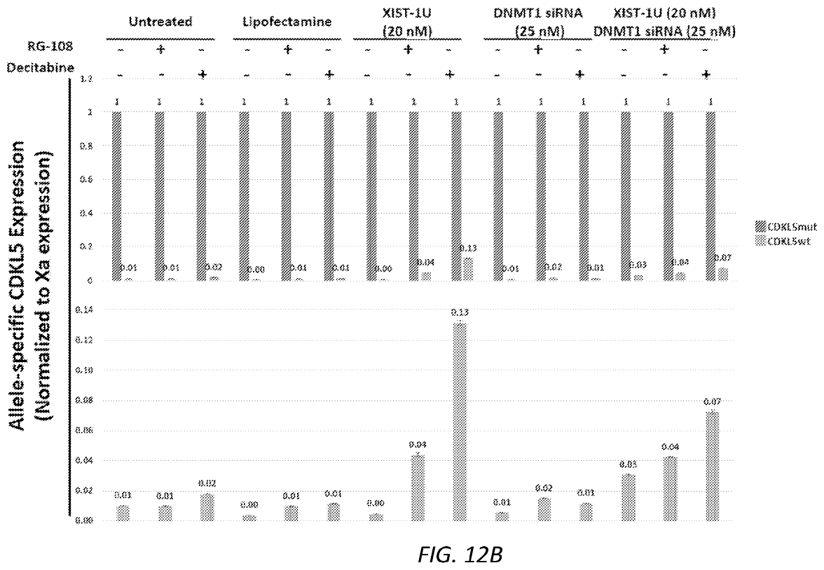

Experiments also showed Xi reactivation with DNMT1 siRNA with and without XIST ASO. A human CDKL5 patient fibroblast line carrying mutation on the Xa was treated with ASO to XIST and siRNAs to DNMT1 SEQ ID NOs: 88-91; see Table 9 below). On day 0, cells were treated with 20 nM of XIST ASO-1U and/or 25 nM of DNMT1 siRNAs with and without 1 uM Decitabine or 1 uM RG-108 (added on days 0, 2, 4, and 6). On day 7, cells were harvested for qPCR analysis. The qPCR results showed a reduction in DNMT1 expression in all experiments using 25 nM of DNMT1 siRNA with or without 20 nM ASO-1U (FIG. 12A). The qPCR results also showed allele specific Xi reactivation (CDKL5 wt allele reactivation) after 7 days treatment. There was an observed 3-7% upregulation of the CDKL5 Xi in fibroblasts when cells were treated with the combination of 25 nM of DNMT1 siRNA and 20 nM ASO-1U with and without Decitabine or RG-108.

TABLE 9
DMNT1 siRNA sequences.

| | |
|---|---|
| SEQ ID NO: 88 | UGGUUUACAUGUCGACUAA |
| SEQ ID NO: 89 | AUAAAUGAAUGGUGGAUCA |
| SEQ ID NO: 90 | CCUGAGCCCUACCGAAUUG |
| SEQ ID NO: 91 | GGACGACCCUGACCUCAAA |

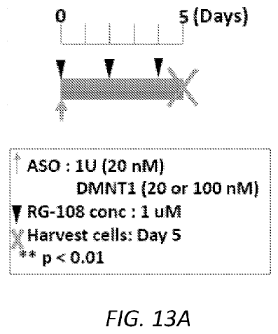

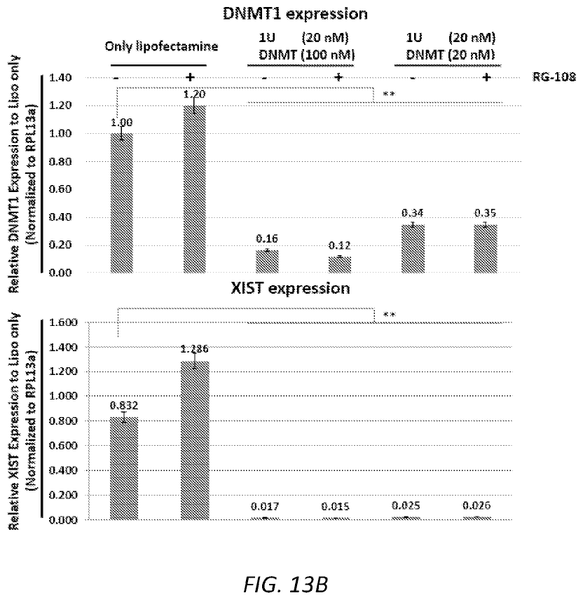

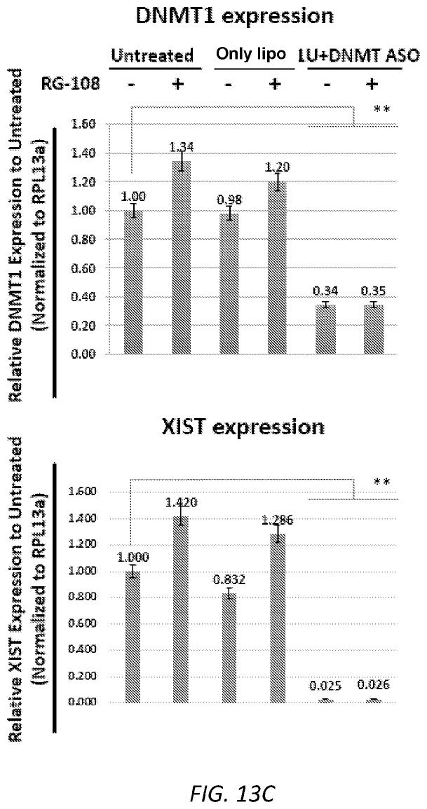

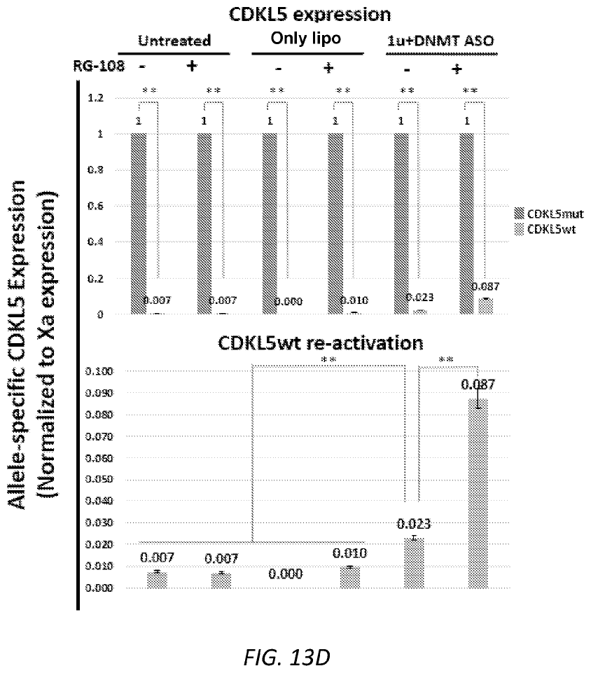

Additional experiments investigating XIST ASO (ASO-1U), DNMT1 ASO, and DNMTi co-treatment were also performed (FIGS. 13A-13D). A human CDKL5 patient fibroblast line carrying mutation on the Xa was treated with ASOs to XIST and DNMT. After 5 days of treatment with 20 nM XIST ASO-1U and 20 nM-100 nM DNMT1 ASO (lipofectamine transfection for both, each added once at day 0) with and without 1.0 uM RG-108 (added every 2 days; days 0, 2, and 4), cells were harvested for qPCR analysis. The qPCR results showed percentage of DNMT1 expression (top bar graph) or XIST expression (bottom bar graph) after 7 days treatment with or without 20 nM XIST ASO-1U, and 20 nM of DNMT1 ASO with and without 1 uM RG-108 (FIGS. 13B and 13C). also showed allele specific Xi reactivation (CDKL5 wt allele reactivation) after 7 days treatment. There was an observed 2-9% upregulation of the CDKL5 Xi in fibroblasts when cells were treated with the combination 10 nM ASO-1U and 10 nM DNMT1 ASO and with or without 1.0 uM RG-108.

These results demonstrate that a combination of an XIST ASO and/or a DNMT1 ASO with DNMTi is an effective method to achieve higher CDKL5 Xi upregulation in human cells. A higher Xi reactivation may have more effective in therapeutic benefit.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated antisense oligonucleotide (ASO) comprising 12-50 consecutive nucleotides of SEQ ID NOs: 1-45, or 12-50 consecutive nucleotides of a sequence at or within 100 nts of the binding sites for ASOs comprising SEQ ID NOs: 1-45 in XIST.

2. The isolated ASO of claim 1, comprising at least one modification.

3. The isolated ASO of claim 2, wherein the at least one modification comprises one or more modified bonds or bases.

4. The isolated ASO of claim 2, wherein the modified bases comprise at least one ribonucleotide, at least one ribonucleotide, at least one deoxyribonucleotide, or at least one bridged nucleotide, wherein the bridged nucleotide is a locked nucleic acid (LNA) nucleotide, a 2′-O-Ethyl (cEt) modified nucleotide, 2′-O-methoxy ethyl (MOE) nucleotide, or a 2′-O,4′-C-ethylene (ENA) modified nucleotide.

5. The isolated ASO of claim 2, wherein the modified bonds comprise phosphorothioate internucleotide linkages between at least two nucleotides, or between all nucleotides.

6. The isolated ASO of claim 2, which is a gapmer or mixmer.

7. The isolated ASO of claim 6, comprising unmodified deoxyribonucleosides in the center flanked by 5′ and 3′ terminal modified nucleosides.

8. The isolated ASO of claim 7, comprising 1, 2, 3, 4, 5, 6, 7, 8, 9, or 10 locked nucleosides at the 3′ end and/or 1, 2, 3, 4, 5, 6, 7, 8, 9, or 10 locked nucleosides at the 5′ end.

9. The isolated ASO of claim 7, comprising 1, 2, 3, 4, 5, 6, 7, 8, 9, or 10 2′-O-methoxy ethyl (MOE) nucleotides at the 3′ end and/or 1, 2, 3, 4, 5, 6, 7, 8, 9, or 10 2′-O-methoxy ethyl (MOE) nucleotides at the 5′ end.

10. The isolated ASO of claim 7, which directs RNAse-H-mediated cleavage of a target XIST transcript.

11. The isolated ASO of claim 7, wherein the locked nucleosides comprise a methylene bridge between the 2′-oxgygen and the 4′-carbon.

12. The isolated ASO of claim 7, comprising one or more the modified bonds, preferably wherein the modified bonds comprise phosphorothioate internucleotide linkages between at least two nucleotides, or between all nucleotides.

13. A composition comprising the isolated ASO of claim 1, and a pharmaceutically acceptable carrier.

14. The composition of claim 13, further comprising an inhibitor of an XIST-interacting protein.

15. The composition of claim 14, wherein the inhibitor of an XIST-interacting protein inhibits a protein selected from SMCla; SMC3; WAPL; RAD21; KIF4; PDS5a/b; CTCF; TOP1; TOP2a; TOP2b; SMARCA4 (BRG1); SMARCA5; SMARCC1; SMARCC2; SMARCB1; RING1a/b (PRC1); PRC2 (EZH2, SUZ12, RBBP7, RBBP4, EED); AURKB; SPEN/MINT/SHARP; DNMT1; DNMT3a/3b; SmcHD1; CTCF; MYEF2; ELAV1; SUN2; Lamin-B Receptor (LBR); LAP; hnRPU/SAF-A; hnRPK; hnRPC; PTBP2; RALY; MATRIN3; MacroH2A; and ATRX.

16. The composition of claim 14, wherein the inhibitor of an XIST-interacting protein is a small molecule inhibitor or an inhibitory nucleic acid that targets a gene encoding the XIST-interacting protein.

17. The composition of claim 14, wherein the inhibitor of an XIST-interacting protein is an inhibitor of DNA methyltransferase (DNMT).

18. The composition of claim 17, wherein the inhibitor of DNMT is RG108, 5-azacytidine, decitabine, Zebularine, procainamide, procaine, psammaplin A, sinefungin, temozolomide, OM173-alphaA, DNMT3A-binding protein, theaflavin 3,3′-digallate, 1- Hydrazinophthalazine, SGI-1027, hydralazine, NSC14778, Olsalazine, Nanaomycin, SID 49645275, Δ²-isoxazoline, epigallocatechin-3-gallate (EGCG), MG98, SGI-110, SGI-1027, SW155246, SW15524601, SW155246-2, or DZNep, an ASO targeting DNMT, optionally comprising SEQ ID NO: 80, TCAAGTTGAGGCCAGAAGGA, or an siRNA targeting DNMT, optionally comprising SEQ ID NOs: 88-91.

19. The composition of claim 14, comprising more than one inhibitor of an XIST-interacting protein.

20. (canceled)

21. A method of increasing expression of an inactive X-linked allele in a cell, preferably a cell of a female heterozygous subject or male hemizygous subject, the method comprising administering to the cell an isolated ASO of claim 1 and an inhibitor of an XIST-interacting protein.

22.-33. (canceled)
